(19) United States
(12) Patent Application Publication
(10) Pub. No.: US 2025/0230241 A1
(43) Pub. Date: Jul. 17, 2025

(54) LOW PH PHARMACEUTICAL ANTIBODY FORMULATION

(71) Applicant: AMGEN INC., Thousand Oaks, CA (US)

(72) Inventors: Twinkle R. Christian, Thousand Oaks, CA (US); Daxian Shan, Thousand Oaks, CA (US)

(21) Appl. No.: 19/022,064

(22) Filed: Jan. 15, 2025

Related U.S. Application Data
(63) Continuation of application No. 16/964,452, filed on Jul. 23, 2020.
(60) Provisional application No. 62/799,577, filed on Jan. 31, 2019.
(60) Provisional application No. 62/628,267, filed on Feb. 8, 2018.

(51) Int. Cl. C07K 16/28; A61K 47/54; A61K 47/60; A61K 47/61

(52) U.S. Cl. CPC C07K 16/2809; A61K 47/542; A61K 47/60; A61K 47/61; C07K 2317/94

(57) ABSTRACT

The present disclosure describes low pH formulations comprising. e.g., an antigen-binding protein that binds CD3, at least one buffer agent, at least one saccharide and at least one surfactant.

### CROSS-REFERENCE TO RELATED APPLICATIONS

The present application claims the benefit of priority to U.S. Provisional Application No. 62/628,267, filed Feb. 8, 2018 and U.S. Provisional Application No. 62/799,577, filed Jan. 31, 2019, the disclosures of which are incorporated herein by reference in their entireties.

### INCORPORATION BY REFERENCE

This application incorporates by reference International Patent Publication No. WO 2016/086196, filed on Nov. 25, 2015; U.S. Patent Publication No. 20160215063, filed on Nov. 25, 2015; International Patent Publication No. WO 2017/091656, filed on Nov. 23, 2016; and U.S. Pat. No. 9,822,186, filed on Mar. 30, 2015, which are expressly incorporated herein by reference in their entirety, with particular reference to the figures, legends and claims therein.

Incorporated by reference in its entirety is a computer-readable nucleotide/amino acid sequence listing submitted concurrently herewith and identified as follows: ASCII (text) file named “52588P_Seqlisting.txt”. 756,646 bytes created Feb. 5, 2019.

### BACKGROUND

Protein-based pharmaceuticals are among the fastest growing therapeutic agents in (pre) clinical development and as commercial products. In comparison with small chemical drugs, protein pharmaceuticals have high specificity and activity at relatively low concentrations, and typically provide for therapy of high impact diseases such as various cancers, auto-immune diseases, and metabolic disorders (Roberts, Trends Biotechnol. 2014 July; 32 (7): 372-80, Wang, Int J Pharm. 1999 Aug. 20; 185 (2): 129-88).

Protein-based pharmaceuticals, such as recombinant proteins, can now be obtained in high purity when first manufactured due to advances in commercial scale purification processes. However, proteins are only marginally stable and are highly susceptible to degradation, both chemical and physical. Chemical degradation refers to modifications involving covalent bonds, such as deamidation, oxidation, cleavage or formation of new disulfide bridges, hydrolysis, isomerization, or deglycosylation. Physical degradation includes protein unfolding, undesirable adsorption to surfaces, and aggregation. Dealing with these physical and chemical instabilities is one of the most challenging tasks in the development of protein pharmaceuticals (Chi et al., Pharm Res, Vol. 20, No. 9, September 2003, pp. 1325-1336, Roberts, Trends Biotechnol. 2014 July; 32 (7): 372-80).

Protein aggregation represents a major event of physical instability of proteins and occurs due to the inherent tendency to minimize the thermodynamically unfavorable interaction between the solvent and hydrophobic protein residues. It is particularly problematic because it is encountered routinely during refolding, purification, sterilization, shipping, and storage processes. Aggregation can occur even under solution conditions where the protein native state is highly thermodynamically favored (e.g., neutral pH and 37° C.) and in the absence of stresses (Chi et al., Pharm Res, Vol. 20, No. 9, September 2003, pp. 1325-1336, Roberts, Trends Biotechnol. 2014 July; 32 (7): 372-80, Wang. Int J Pharm. 1999 Aug. 20; 185 (2): 129-88, Mahler J Pharm Sci. 2009 September; 98 (9): 2909-34.).

Also half-life extended antibody constructs such as bispecific T cell engagers comprising a half-life extending modality such as Fc-molecules have to be protected against protein aggregation and/or other degradation events. Protein aggregation is problematic because it can impair biological activity of the therapeutic proteins. Moreover, aggregation of proteins leads to undesirable aesthetics of the drug product, and decreases product yield due to elaborate purification steps that are required to remove the aggregates from the end product. More recently, there has also been growing concern and evidence that the presence of aggregated proteins (even humanized or fully human proteins) can significantly increase the risk that a patient will develop an immune response to the active protein monomer, resulting in the formation of neutralizing antibodies and drug resistance, or other adverse side effects (Mahler J Pharm Sci. 2009 September; 98 (9): 2909-34).

In general, several efforts have been reported in the literature to minimize protein aggregation by various mechanisms. Proteins can be stabilized and thus protected from aggregate formation and other chemical changes by modifying their primary structure, thereby increasing interior hydrophobicity and reducing outer hydrophobicity. However, genetic engineering of proteins may result in impaired functionality and/or increased immunogenicity. Another approach focuses on the dissociation of aggregates (referred to as “disaggregation”) to recover functional, native monomers by using various mechanisms such as temperature, pressure, pH, and salts. Currently, protein aggregates are removed as impurities mainly in the polishing steps of downstream processing. However, in cases of high levels of high-molecular weight (HMW), removing significant amount of HMW not only results in substantial yield loss but also makes the design of a robust downstream process challenging (Chi et al., Pharm Res, Vol. 20, No. 9, September 2003, pp. 1325-1336).

Preserving protein stability and activity in biological and biotechnological applications poses serious challenges. There is a need in the art for optimized pharmaceutical compositions that provide for enhanced stabilization of therapeutic proteins and reduce aggregation and denaturation or degradation during formulation, filling, shipping, storage and administration, thereby preventing loss-of-function and adverse immunogenic reactions.

### SUMMARY

In one aspect, described herein is a pharmaceutical composition comprising an antigen-binding protein described herein, at least one buffer, at least one surfactant and at least one saccharide, wherein the pH of the pharmaceutical composition ranges from 3.5 to 5.

In some embodiments, the antigen-binding protein is an antibody. In some embodiments, the antibody is a bispecific antibody, such as a bispecific antibody that binds CD3.

In some embodiments, antigen-binding protein is a heterodimeric antibody that binds CD3. In some embodiments, the heterodimeric antibody comprises a) a first monomer comprising a first Fc domain and an anti-CD3 scFv comprising (i) a scFv variable light domain comprising vlCDR1 as set forth in SEQ ID NO: 15, vlCDR2 as set forth in SEQ ID NO: 16, and vlCDR3 as set forth in SEQ ID NO: 17, and (ii) a scFv variable heavy domain comprising vhCDR1 as set forth in SEQ ID NO: 11, vhCDR2 as set forth in SEQ ID NO: 12, and vhCDR3 as set forth in SEQ ID NO: 13, wherein said scFv is covalently attached to the N-terminus of said Fc domain using a domain linker; b) a second monomer comprising i) an anti-CD38 heavy variable domain comprising vhCDR1 as set forth in SEQ ID NO: 65, vhCDR2 as set forth in SEQ ID NO: 66, and vhCDR3 as set forth in SEQ ID NO: 67, and ii) a heavy constant domain comprising a second Fc domain; and c) a light chain comprising a constant domain and an anti-CD38 variable light domain comprising vlCDR1 as set forth in SEQ ID NO: 69, vlCDR2 as set forth in SEQ ID NO: 70, and vlCDR3 as set forth in SEQ ID NO: 71; and wherein the pH of the pharmaceutical composition ranges from 3.5 to 5.

In some embodiments, the anti-CD3 scFv comprises an amino acid sequence at least 90%, at least 95% or 100% identical to the amino acid sequence set forth in SEQ ID NO: 18.

In some embodiments, the anti-CD38 variable light domain comprises an amino acid sequence at least 90%, at least 95% or 100% identical to the amino acid sequence set forth in SEQ ID NO: 68.

In some embodiments, the anti-CD38 heavy variable domain comprises an amino acid sequence at least 90%, at least 95% or 100% identical to the amino acid sequence set forth in SEQ ID NO: 64.

In some embodiments, the first monomer comprises an amino acid sequence at least 90%, at least 95% or 100% identical to the amino acid sequence set forth in SEQ ID NO: 335.

In some embodiments, the second monomer comprises an amino acid sequence at least 90%, at least 95% or 100% identical to the amino acid sequence set forth in SEQ ID NO: 337.

In some embodiments, the light chain comprises an amino acid sequence at least 90%, at least 95% or 100% identical to the amino acid sequence set forth in SEQ ID NO: 336.

In various aspects, the antigen-binding protein is a heterodimeric antibody comprising a) a first monomer comprising a first heavy chain comprising: 1) a first variable heavy domain; 2) a first constant heavy chain comprising a first CH1 domain and a first Fc domain; and 3) a scFv that binds human CD3 and comprises (i) a scFv variable light domain comprising vlCDR1 set forth in SEQ ID NO:387, vlCDR2 set forth in SEQ ID NO: 388, and vlCDR3 set forth in SEQ ID NO: 389, (ii) a scFv linker, and (iii) a scFv variable heavy domain comprising vhCDR1 set forth in SEQ ID NO: 383, vhCDR2 set forth in SEQ ID NO: 384, and vhCDR3 set forth in SEQ ID NO: 385; wherein said scFv is covalently attached between the C-terminus of said CH1 domain and the N-terminus of said first Fc domain using domain linker(s). The heterodimeric antibody further comprises b) a second monomer comprising a second heavy chain comprising a second variable heavy domain and a second constant heavy chain comprising a second Fc domain; and c) a common light chain comprising a variable light domain and a constant light domain. The first variable heavy domain and the variable light domain bind human STEAP1, and the second variable heavy domain and the variable light domain bind human STEAP1. The first variable heavy domain and the second variable heavy domain comprises heavy chain CDRs comprising vhCDR1 set forth in SEQ ID NO: 360, vhCDR2 set forth in SEQ ID NO: 361 or SEQ ID NO: 363, and vhCDR3 set forth in SEQ ID NO: 362, and the variable light domain comprises light chain CDRs comprising vlCDR1 set forth in SEQ ID NO: 357, vlCDR2 set forth in SEQ ID NO: 358, and vlCDR3 set forth in SEQ ID NO: 359. Alternatively, the first variable heavy domain and the second variable heavy domain comprise heavy chain CDRs comprising vhCDR1 set forth in SEQ ID NO: 368, vhCDR2 set forth in SEQ ID NO: 369, and vhCDR3 set forth in SEQ ID NO: 370, and the variable light domain comprises light chain CDRs comprising vlCDR1 set forth in SEQ ID NO: 371, vlCDR2 set forth in SEQ ID NO: 372, and vlCDR3 set forth in SEQ ID NO: 373. In various embodiments, the first variable heavy domain and the second variable heavy domain comprise an amino acid sequence at least 90% identical (e.g., at least 95% identical or 100% identical) to SEQ ID NO: 377 or 379 and/or the variable light domain comprises an amino acid sequence at least 90% identical (e.g., at least 95% identical or 100% identical) to SEQ ID NO: 378. Alternatively, the first variable heavy domain and the second variable heavy domain comprise an amino acid sequence at least 90% identical (e.g., at least 95% identical or 100% identical) to SEQ ID NO: 380 and/or the variable light domain comprises an amino acid sequence at least 90% identical (e.g., at least 95% identical or 100% identical) to SEQ ID NO: 381. The scFv optionally comprises a variable heavy region and a variable light region of SEQ ID NO: 382 and SEQ ID NO:383. The scFv linker optionally comprises SEQ ID NO: 391. In various aspects, the scFv comprises the sequence of SEQ ID NO: 390. In various aspects, a) the first monomer comprises the sequence of SEQ ID NO: 366 or SEQ ID NO: 367, the second monomer comprises the sequence of SEQ ID NO:365, and the common light chain comprises the sequence of SEQ ID NO:364; or b) the first monomer comprises the sequence of SEQ ID NO: 376, the second monomer comprises the sequence of SEQ ID NO: 375, and the common light chain comprises the sequence of SEQ ID NO:374.

The pharmaceutical composition of the disclosure comprises at least one buffer agent. In some embodiments, the buffer agent is an acid selected from the group consisting of acetate, glutamate, citrate, succinate, tartrate, fumarate, maleate, histidine, phosphate, and 2-(N-morpholino)ethanesulfonate or a combination thereof. In some embodiments, the at least one buffer agent is present in the composition at a concentration ranging from about 5 mM to about 200 mM (or about 10 mM to about 50 mM).

The pharmaceutical composition of the disclosure comprises at least one saccharide. In some embodiments, the saccharide is selected from the group consisting of monosaccharide, disaccharide, cyclic polysaccharide, sugar alcohol, linear branched dextran, and linear non-branched dextran. In some embodiments, the saccharide is a sugar alcohol (e.g., sorbitol). In some embodiments, the saccharide is a disaccharide selected from the group consisting of sucrose, trehalose, mannitol, and sorbitol or a combination thereof. In some embodiments, the at least one saccharide is present in the composition at a concentration ranging from about 1 to about 15% (w/V) (or about 9 to about 12% (w/V) or about 5% to about 12% (w/V) or about 7% to about 12% (w/V)).

The pharmaceutical composition of the disclosure comprises at least one surfactant. In some embodiments, the surfactant is selected from the group consisting of polysorbate 20, polysorbate 40, polysorbate 60, polysorbate 80, poloxamer 188, pluronic F68, triton X-100, polyoxyethylen3, and PEG 3350, PEG 4000, or a combination thereof. In some embodiments, the at least one surfactant is present in the composition at a concentration ranging from 0.004 to about 0.5% (w/V) (or about 0.001 to about 0.01% (w/V), or about 0.001 to about 0.5% (w/V) or about 0.004 to about 0.01% (w/V)).

In some embodiments, the pH of the composition ranges from 4.0 to 5.0. In some embodiments, the pH of the composition is 4.2.

In some embodiments, the composition has an osmolarity in the range of about 150 to about 500 mOsm.

The pharmaceutical compositions of the disclosure may optionally further comprise an excipient selected from the group consisting of a polyol and an amino acid. In some embodiments, the excipient is present at a concentration ranging from about 0.1 to about 15% (w/V).

The pharmaceutical composition, in some embodiments, comprises 10 mM glutamate, 9% (w/V) sucrose and 0.01% (w/V) polysorbate 80, and wherein the pH of the liquid pharmaceutical composition is 4.2. In some embodiments, the heterodimeric antibody is present in the composition at a concentration ranging from about 0.1 mg/mL to about 8 mg/mL. In some embodiments, the heterodimeric antibody is present in the composition at a concentration ranging from about 0.1 mg/mL to about 20 mg/mL. In some embodiments, the heterodimeric antibody is present in the composition at a concentration of 1 mg/mL, 5 mg/mL, 10 mg/mL or 20 mg/mL. In some embodiments, the heterodimeric antibody is present in the composition in an amount ranging from about 50 μg to about 200 mg.

The pharmaceutical compositions of the disclosure can be a lyophilized composition or a liquid composition. In some embodiments, the pharmaceutical composition is a lyophilized composition or a reconstituted lyophilized composition.

In another aspect, described herein is a method of treating cancer in a subject in need thereof comprising administering a composition of the disclosure to the subject. In some embodiments, the cancer is multiple myeloma. In some embodiments, the cancer is prostate cancer.

It should be understood that while various embodiments in the specification are presented using “comprising” language, under various circumstances, a related embodiment may also be described using “consisting of” or “consisting essentially of” language. The disclosure contemplates embodiments described as “comprising” a feature to include embodiments which “consist of” the feature. It is to be noted that the term “a” or “an” refers to one or more, for example, “an immunoglobulin molecule,” is understood to represent one or more immunoglobulin molecules. As such, the terms “a” (or “an”), “one or more,” and “at least one” can be used interchangeably herein.

It should also be understood that when describing a range of values, the characteristic being described could be an individual value found within the range. For example, “a pH from about pH 4 to about pH 6,” could be, but is not limited to, pH 4, 4.2, 4.6, 5.1, 5.5 etc. and any value in between such values. Additionally, “a pH from about pH 4 to about pH 6,” should not be construed to mean that the pH of a formulation in question varies 2 pH units in the range from pH 4 to pH 6 during storage, but rather a value may be picked in that range for the pH of the solution, and the pH remains buffered at about that pH. In some embodiments, when the term “about” is used, it means the recited number plus or minus 5%, 10%, 15% or more of that recited number. The actual variation intended is determinable from the context.

In any of the ranges described herein, the endpoints of the range are included in the range. However, the description also contemplates the same ranges in which the lower and/or the higher endpoint is excluded. Additional features and variations of the invention will be apparent to those skilled in the art from the entirety of this application, including the drawing and detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

All references cited herein are hereby incorporated by reference.

### BRIEF DESCRIPTION OF THE FIGURES

FIGS. 1A and 1B depict several formats of heterodimeric antibodies. Two forms of the “bottle opener” format are depicted, one with the anti-CD3 antigen binding domain comprising a scFv and the anti-CD38 antigen binding domain comprising a Fab (as examples of antigen-binding domains), and one with these reversed. The mAb-Fv, mAb-scFv, Central-scFv (or “XmAb²⁺¹” format) and Central-Fv formats are all shown. In addition, “one-armed” formats, where one monomer just comprises an Fc domain are shown, both a one arm Central-scFv and a one arm Central-Fv. A dual scFv format is also shown.

FIG. 2 depicts the sequences of the “High-Int #1″Anti-CD3_H1.32_L1.47 construct, including the variable heavy and light domains (CDRs underlined), as well as the individual vl and vhCDRs, as well as an scFv construct with a charged linker (double underlined). As is true of all the sequences depicted in the Figures, this charged linker may be replaced by an uncharged linker or a different charged linker, as needed.

FIG. 3 depicts the sequences of the intermediate CD38: OKT10_HIL1.24 construct, including the variable heavy and light domains (CDRs underlined), as well as the individual vl and vhCDRs, as well as an scFv construct with a charged linker (double underlined).

FIG. 4 depicts the sequences of the Low CD38: OKT10_HIL1 construct, including the variable heavy and light domains (CDRs underlined), as well as the individual vl and vhCDRs, as well as an scFv construct with a charged linker (double underlined).

FIG. 5 depicts the sequences of XENP18971. CDRs are underlined.

FIG. 6 depicts the sequences of XENP18969. CDRs are underlined.

FIG. 7 depicts the sequence of human CD3 & (SEQ ID NO: 130).

FIG. 8 depicts the full length (SEQ ID NO:131) and extracellular domain (ECD; SEQ ID NO:132) of the human CD38 protein.

FIGS. 9A-9E depict useful pairs of heterodimerization variant sets (including skew and pI variants).

FIG. 10 depicts a list of isosteric variant antibody constant regions and their respective substitutions. pI_(−) indicates lower pI variants, while pI_(+) indicates higher pI variants. These can be optionally and independently combined with other heterodimerization variants of the invention (and other variant types as well, as outlined herein).

FIG. 11 depicts useful ablation variants that ablate FcγR binding (sometimes referred to as “knock outs” or “KO” variants).

FIG. 12 shows two embodiments of antibodies of the disclosure.

FIGS. 13A and 13B depict a number of charged scFv linkers that find use in increasing or decreasing the pI of heterodimeric antibodies that utilize one or more scFv as a component. A single prior art scFv linker with a single charge is referenced as “Whitlow,” from Whitlow et al., Protein Engineering 6 (8): 989-995 (1993). It should be noted that this linker was used for reducing aggregation and enhancing proteolytic stability in scFvs.

FIG. 14 depicts a list of engineered heterodimer-skewing Fc variants with heterodimer yields (determined by HPLC-CIEX) and thermal stabilities (determined by DSC). Not determined thermal stability is denoted by “n.d.”

FIGS. 15A and 15B depict stability-optimized, humanized anti-CD3 variant scFvs. Substitutions are given relative to the H1_L1.4 scFv sequence. Amino acid numbering is Kabat numbering.

FIGS. 16A and 16B depict amino acid sequences of stability-optimized, humanized anti-CD3 variant scFvs. CDRs are underlined. For each heavy chain/light chain combination, four sequences are listed: (i) scFv with C-terminal 6×His tag, (ii) scFv alone, (iii) VH alone, (iv) VL alone.

FIG. 17 depicts the sequences of XENP18971. CDRs are underlined.

FIG. 18 depicts the sequences of XENP18969. CDRs are underlined.

FIG. 19 shows a matrix of possible combinations of embodiments. An “A” means that the CDRs of the referenced CD3 sequences can be combined with the CDRs of CD38 construct on the left hand side. That is, for example for the top left hand cell, the vhCDRs from the variable heavy chain CD3 H1.30 sequence and the vlCDRs from the variable light chain of CD3 L1.47 sequence can be combined with the vhCDRs from the CD38 OKT10 H1.77 sequence and the vlCDRs from the OKT10L1.24 sequence. A “B” means that the CDRs from the CD3 constructs can be combined with the variable heavy and light domains from the CD38 construct. That is, for example for the top left hand cell, the vhCDRs from the variable heavy chain CD3 H1.30 sequence and the vlCDRs from the variable light chain of CD3 L1.47 sequence can be combined with the variable heavy domain CD38 OKT10 H1.77 sequence and the OKT10L1.24 sequence. A “C” is reversed, such that the variable heavy domain and variable light domain from the CD3 sequences are used with the CDRs of the CD38 sequences. A “D” is where both the variable heavy and variable light chains from each are combined. An “E” is where the scFv of the CD3 is used with the CDRs of the CD38 antigen binding domain construct, and an “F” is where the scFv of the CD3 is used with the variable heavy and variable light domains of the CD38 antigen binding domain.

FIG. 20 depicts the sequences of XmAb18968, also referenced herein as Antibody A. CDRs are underlined.

FIG. 21 is a table associating various CDR sequences, variable region sequences, heavy and light chain sequences, scFv sequences, backbone sequences, etc., with sequence identifiers set forth in the sequence listing accompanying the instant application. Regarding the several bottle opener format backbones noted (SEQ ID NOs: 347-354), the sequences are provided without the Fv sequences (e.g., the scFv and the vh and vl for the Fab side). As will be appreciated by those in the art and outlined below, these sequences can be used with any vh and vl pairs outlined herein, with one monomer including a scFv (optionally including a charged scFv linker) and the other monomer including the Fab sequences (e.g., a vh attached to the “Fab side heavy chain” and a vl attached to the “constant light chain”). The scFv can be anti-CD3 or anti-CD38, with the Fab being the other. (“Fab” referring to the portion that comprises the VH, CH1, VL, and CL immunoglobulin domains.) That is, for example, any Fv sequences outlined herein for CD3 and CD38 can be incorporated into these backbones in any combination.

FIGS. 22A and 22B show the main peak loss of Antibody A when formulated in Formulation A (FIG. 22A) and Formulation B (FIG. 22B) as determined by SE-UHPLC.

FIG. 23 shows the main peak loss of Antibody A when formulated in Formulation C as determined by SE-UHPLC.

FIGS. 24A and 24B show the main peak loss of Antibody A when formulated in Formulation A (FIG. 24A) and Formulation B (FIG. 24B) as determined by CE-HPLC.

FIG. 25 shows the main peak loss of Antibody A when formulated in Formulation C as determined by CE-HPLC.

FIG. 26 shows a main peak loss of Antibody A at −30° C. when formulated in Formulation A as determined by rCE.

FIG. 27 shows a main peak loss of Antibody A at 40° C. when formulated in Formulation B as determined by rCE.

FIG. 28 shows a main peak loss of Antibody A when formulated in Formulation C as determined by rCE.

FIG. 29 shows the percent deamidation of Antibody A when formulated in Formulations B and C at 4° C., 2° C., and 40° C. for three months as determined by MAM.

FIG. 30 shows the percent deamidation of Antibody A when formulated in Formulation D at 40° C. for 0 weeks, two weeks and four weeks as determined by MAM.

FIG. 31 shows the percent deamidation of Antibody A when formulated in Formulations B and C at 40° C. for 1 month as determined by MAM.

### DETAILED DESCRIPTION

The present disclosure describes low pH formulations comprising an antigen-binding protein that binds CD3.

In some embodiments, the antigen-binding protein is an antibody, such as a bispecific antibody (e.g., a bispecific antibody that binds CD3). In some embodiments, the antigen-binding protein is a heterodimeric antibody that co-engages CD3 and CD38 in such a manner so as to transiently connect malignant cells with T cells, thereby inducing T cell mediated killing of the bound malignant cell. In other embodiments, the antigen-binding protein is a heterodimeric antibody that co-engages CD3 and STEAP1.

Specific protein-based pharmaceuticals are not stable in liquid formulations over a longer period of time and especially not refrigeration temperature 4° C. and above. A general concept underlying the present invention is the finding that colloidal stability of a liquid pharmaceutical composition comprising an antigen-binding protein according to the present invention is improved at low pH.

Various aspects of the formulation are described below. The use of section headings are merely for the convenience of reading, and not intended to be limiting per se. The entire document is intended to be viewed as a unified disclosure, and it should be understood that all combinations of features described herein are contemplated.

In one aspect, described herein is a pharmaceutical composition comprising an antigen-binding protein described herein, at least one buffer, at least one surfactant and at least one saccharide, wherein the pH of the pharmaceutical composition ranges from 3.5 to 5.

### Buffers

The pharmaceutical composition of the invention comprises a buffer, which optionally may be selected from the group consisting of acetate, glutamate, citrate, succinate, tartrate, fumarate, maleate, histidine, phosphate, 2-(N-morpholino)ethanesulfonate, potassium phosphate, acetic acid/sodium acetate, citric acid/sodium citrate, succinic acid/sodium succinate, tartaric acid/sodium tartrate, histidine/histidine HCl, glycine, Tris, glutamate, and combinations thereof. In some embodiments, the pharmaceutical composition comprises at least one buffer selected from the group consisting of acetate, glutamate, citrate, succinate, tartrate, fumarate, maleate, histidine, phosphate, 2-(N-morpholino)ethanesulfonate and combinations thereof.

Buffering agents are often employed to control pH in the formulation. In some embodiments, the buffer is added in a concentration that maintains pH of the formulation of about 3.5 to 5, or about 4 to 5, or about 4.2. The effect of pH on formulations may be characterized using any one or more of several approaches such as accelerated stability studies and calorimetric screening studies (Remmele R. L. Jr., et al., Biochemistry, 38 (16): 5241-7 (1999)).

Organic acids, phosphates and Tris are suitable buffers in protein formulations (Table 1). The buffer capacity of the buffering species is maximal at a pH equal to the pKa and decreases as pH increases or decreases away from this value. Ninety percent of the buffering capacity exists within one pH unit of its pKa. Buffer capacity also increases proportionally with increasing buffer concentration.

Several factors are typically considered when choosing a buffer. For example, the buffer species and its concentration should be defined based on its pKa and the desired formulation pH. Also important is to ensure that the buffer is compatible with the protein drug, other formulation excipients, and does not catalyze any degradation reactions. Recently, polyanionic carboxylate buffers such as citrate and succinate have been shown to form covalent adducts with the side chain residues of proteins. A third aspect to be considered is the sensation of stinging and irritation the buffer may induce. For example, citrate is known to cause stinging upon injection (Laursen T, et al., Basic Clin Pharmacol Toxicol., 98 (2): 218-21 (2006)). The potential for stinging and irritation is greater for drugs that are administered via the SC or IM routes, where the drug solution remains at the site for a relatively longer period of time than when administered by the IV route where the formulation gets diluted rapidly into the blood upon administration. For formulations that are administered by direct IV infusion, the total amount of buffer (and any other formulation component) needs to be monitored. For example, it has been reported that potassium ions administered in the form of the potassium phosphate buffer, can induce cardiovascular effects in a patient (Hollander-Rodriguez J C, et al., Am. Fam. Physician., 73 (2): 283-90 (2006)).

TABLE 1
Buffering agents and their pKₐ values

| Buffer | pKₐ | Example drug product |
|---|---|---|
| Acetate | 4.8 | Neupogen, Neulasta |
| Succinate | pKₐ₁ = 4.8, pKₐ₂ = 5.5 | Actimmune |
| Citrate | pKₐ₁ = 3.1, pKₐ₂ = 4.8, | Humira |
| | pKₐ₃ = 6.4 | |
| Histidine | 6.0 | Xolair |
| (imidazole) | | |
| Phosphate | pKₐ₁ = 2.15, pKₐ₂ = 7.2, | Enbrel (liquid formulation) |
| | pKₐ₃ = 12.3 | |
| Tris | 8.1 | Leukine |

The buffer system present in the formulation is selected to be physiologically compatible and to maintain a desired pH.

The buffer may be present in any amount suitable to maintain the pH of the formulation at a predetermined level. The buffer may be present at a concentration between about 0.1 mM and about 1000 mM (1 M), or between about 5 mM and about 200 mM, or between about 5 mM to about 100 mM, or between about 10 mM and 50 about mM. Suitable buffer concentrations encompass concentrations of about 200 mM or less. In some embodiments, the buffer in the formulation is present in a concentration of about 190 mM, about 180 mM, about 170 mM, about 160 mM, about 150 mM, about 140 mM, about 130 mM, about 120 mM, about 110 mM, about 100 mM, about 80 mM, about 70 mM, about 60 mM, about 50 mM, about 40 mM, about 30 mM, about 20 mM, about 10 mM or about 5 mM. In some embodiments, the concentration of the buffer is at least 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, 90, 100, 200, 500, 700, or 900 mM. In some embodiments, the concentration of the buffer is between 1, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, or 90 mM and 100 mM. In some embodiments, the concentration of the buffer is between 5, 6, 7, 8, 9, 10, 11, 12, 13, 14, 15, 16, 17, 18, 19, 20, 30, or 40 mM and 50 mM. In some embodiments, the concentration of the buffer is about 10 mM.

Other exemplary pH buffering agents used to buffer the formulation as set out herein include, but are not limited to glycine, glutamate, succinate, phosphate, acetate, and aspartate. Amino acids such as histidine and glutamic acid can also be used as buffering agents.

### Surfactants

The pharmaceutical compositions described here comprise at least one surfactant. Surfactants are commonly used in protein formulations to prevent surface-induced degradation. Surfactants are amphipathic molecules with the capability of out-competing proteins for interfacial positions. Hydrophobic portions of the surfactant molecules occupy interfacial positions (e.g., air/liquid), while hydrophilic portions of the molecules remain oriented towards the bulk solvent. At sufficient concentrations (typically around the detergent's critical micellar concentration), a surface layer of surfactant molecules serve to prevent protein molecules from adsorbing at the interface. Thereby, surface-induced degradation is minimized. Surfactants include, e.g., fatty acid esters of sorbitan polyethoxylates, i.e., polysorbate 20 and polysorbate 80 (see e.g., Avonex®, Neupogen®, Neulasta®). The two differ only in the length of the aliphatic chain that imparts hydrophobic character to the molecules, C-12 and C-18, respectively. Accordingly, polysorbate-80 is more surface-active and has a lower critical micellar concentration than polysorbate-20. The surfactant poloxamer 188 has also been used in several marketed liquid products such Gonal-F®, Norditropin®, and Ovidrel®.

Detergents can also affect the thermodynamic conformational stability of proteins. Here again, the effects of a given excipient will be protein specific. For example, polysorbates have been shown to reduce the stability of some proteins and increase the stability of others. Detergent destabilization of proteins can be rationalized in terms of the hydrophobic tails of the detergent molecules that can engage in specific binding with partially or wholly unfolded protein states. These types of interactions could cause a shift in the conformational equilibrium towards the more expanded protein states (i.e., increasing the exposure of hydrophobic portions of the protein molecule in complement to binding polysorbate). Alternatively, if the protein native state exhibits some hydrophobic surfaces, detergent binding to the native state may stabilize that conformation.

Another aspect of polysorbates is that they are inherently susceptible to oxidative degradation. Often, as raw materials, they contain sufficient quantities of peroxides to cause oxidation of protein residue side-chains, especially methionine. The potential for oxidative damage arising from the addition of stabilizer emphasizes the point that the lowest effective concentrations of excipients should be used in formulations. For surfactants, the effective concentration for a given protein will depend on the mechanism of stabilization. It has been postulated that if the mechanism of surfactant stabilization is related to preventing surface-denaturation the effective concentration will be around the detergent's critical micellar concentration. Conversely, if the mechanism of stabilization is associated with specific protein-detergent interactions, the effective surfactant concentration will be related to the protein concentration and the stoichiometry of the interaction (Randolph T. W., et al., Pharm Biotechnol., 13:159-75 (2002)).

Surfactants may also be added in appropriate amounts to prevent surface related aggregation phenomenon during freezing and drying (Chang, B. J. Pharm. Sci. 85:1325, (1996)). Exemplary surfactants include anionic, cationic, nonionic, zwitterionic, and amphoteric surfactants including surfactants derived from naturally-occurring amino acids. Anionic surfactants include, but are not limited to, sodium lauryl sulfate, dioctyl sodium sulfosuccinate and dioctyl sodium sulfonate, chenodeoxycholic acid, N-lauroylsarcosine sodium salt, lithium dodecyl sulfate, 1-octanesulfonic acid sodium salt, sodium cholate hydrate, sodium deoxycholate, and glycodeoxycholic acid sodium salt. Cationic surfactants include, but are not limited to, benzalkonium chloride or benzethonium chloride, cetylpyridinium chloride monohydrate, and hexadecyltrimethylammonium bromide. Zwitterionic surfactants include, but are not limited to, CHAPS, CHAPSO, SB3-10, and SB3-12. Non-ionic surfactants include, but are not limited to, digitonin, Triton X-100, Triton X-114, TWEEN-20, and TWEEN-80. In another embodiment, surfactants include lauromacrogol 400; polyoxyl 40 stearate; polyoxyethylene hydrogenated castor oil 10, 40, 50 and 60; glycerol monostearate; polysorbate 40, 60, 65 and 80; soy lecithin and other phospholipids such as DOPC, DMPG, DMPC, and DOPG; sucrose fatty acid ester; methyl cellulose and carboxymethyl cellulose.

Pharmaceutical compositions described herein comprise at least one surfactant, either individually or as a mixture in different ratios. In some embodiments, the composition comprises a surfactant at a concentration of about 0.001% to about 5% w/v (or about 0.004 to about 0.5% w/v or about 0.001 to about 0.01% w/v or about 0.004 to about 0.01% w/v). In some embodiments, the composition comprises a surfactant at a concentration of at least 0.001, at least 0.002, at least 0.003, at least 0.004, at least 0.005, at least 0.007, at least 0.01, at least 0.05, at least 0.1, at least 0.2, at least 0.3, at least 0.4, at least 0.5, at least 0.6, at least 0.7, at least 0.8, at least 0.9, at least 1.0, at least 1.5, at least 2.0, at least 2.5, at least 3.0, at least 3.5, at least 4.0, or at least 4.5% w/v. In some embodiments, the composition comprises a surfactant at a concentration of about 0.004% to about 0.5% w/v. In some embodiments, the composition comprises a surfactant at a concentration of about 0.004 to about 0.5% w/v. In some embodiments, the composition comprises a surfactant at a concentration of about 0.001 to about 0.01% w/v. In some embodiments, the composition comprises a surfactant at a concentration of about 0.004 to about 0.01% w/v. In some embodiments, the composition comprises a surfactant at a concentration of about 0.004, about 0.005, about 0.007, about 0.01, about 0.05, about 0.1, about 0.2, about 0.3, about 0.4% w/v to about 0.5% w/v. In some embodiments, the composition comprises a surfactant incorporated in a concentration of about 0.001% to about 0.01% w/v. In some embodiments, the surfactant is polysorbate 80 and the polysorbate 80 is present in a concentration of about 0.01% w/v.

### Saccharides

The pharmaceutical compositions described herein comprise at least one saccharide. A saccharide can be added as a stabilizer or a bulking agent. The term “stabilizer” as used herein refers to an excipient capable of preventing aggregation or other physical degradation, as well as chemical degradation (for example, autolysis, deamidation, oxidation, etc.) in an aqueous and solid state. Stabilizers that are employed in pharmaceutical compositions include, but are not limited to, sucrose, trehalose, mannose, maltose, lactose, glucose, raffinose, cellobiose, gentiobiose, isomaltose, arabinose, glucosamine, fructose, mannitol, sorbitol, glycine, arginine HCL, poly-hydroxy compounds, including polysaccharides such as dextran, starch, hydroxyethyl starch, cyclodextrins, N-methyl pyrollidene, cellulose and hyaluronic acid, and sodium chloride (Carpenter et al., Develop. Biol. Standard 74:225, (1991)).

In some embodiments, the at least one saccharide is selected from the group consisting of monosaccharide, disaccharide, cyclic polysaccharide, sugar alcohol, linear branched dextran, and linear non-branched dextran, and combinations thereof. In some embodiments, the at least one saccharide is a disaccharide selected from the group consisting of sucrose, trehalose, mannitol, and sorbitol or a combination thereof.

In some embodiments, the pharmaceutical composition comprises at least one saccharide at a concentration of about 0.01% to about 40% w/v, or about 00.1% to about 20% w/v, or about 1% to about 15% w/v. In some embodiments, the pharmaceutical composition comprises at least one saccharide at a concentration of at least 0.5, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, or at least 40% w/v. In some embodiments, the pharmaceutical composition comprises at least one saccharide at a concentration of about 1, about 2, about 3, about 4, about 5, about 6, about 7, about 8, about 9, about 10, about 11, about 12, about 13, about 14% to about 15% w/v. In some embodiments, the pharmaceutical composition comprises at least one saccharide at a concentration of about 1% to about 15% w/v. In a yet further embodiment, the pharmaceutical composition comprises at least one saccharide at a concentration of about 9%, about 9.5%, about 10%, about 10.5%, about 11%, about 11.5%, or about 12% w/v. In some embodiments, the pharmaceutical composition comprises at least one saccharide at a concentration of about 9% to about 12% w/v. In some embodiments, the at least one saccharide is in the composition at a concentration of about 9% w/v. In some embodiments, the at least one saccharide is selected from the group consisting of sucrose, trehalose, mannitol and sorbitol or a combination thereof. In some embodiments, the saccharide is sorbitol and is present in the composition ranging from about 9% to about 12% w/v.

If desired, the formulations also include appropriate amounts of bulking and osmolarity regulating agents, such as a saccharide, suitable for forming a lyophilized “cake.”

In a preferred embodiment, the pharmaceutical composition comprises 10 mM glutamate, 9% (w/V) sucrose and 0.01% (w/V) polysorbate 80, wherein the pH of the pharmaceutical composition is 4.2.

### Other Considerations

As used herein, the term “pharmaceutical composition” relates to a composition which is suitable for administration to a subject in need thereof. The terms “subject” or “individual” or “animal” or “patient” are used interchangeably herein to refer to any subject, particularly a mammalian subject, for whom administration of the pharmaceutical composition of the invention is desired. Mammalian subjects include humans, non-human primates, dogs, cats, guinea pigs, rabbits, rats, mice, horses, cattle, cows, and the like, with humans being preferred. The pharmaceutical composition of the present invention is stable and pharmaceutically acceptable, i.e., capable of eliciting the desired therapeutic effect without causing significant undesirable local or systemic effects in the subject to which the pharmaceutical composition is administered. Pharmaceutically acceptable compositions of the invention may be sterile and/or pharmaceutically inert. Specifically, the term “pharmaceutically acceptable” can mean approved by a regulatory agency or other generally recognized pharmacopoeia for use in animals, and more particularly in humans.

The formulation provided by the disclosure comprises an antigen-binding protein (e.g., heterodimeric antibody) described herein. In some embodiments, the heterodimeric antibody is provided in a therapeutically effective amount. By “therapeutically effective amount” is meant an amount of said heterodimeric antibody that elicits the desired therapeutic effect. Therapeutic efficacy and toxicity can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, ED50/LD50. Formulations that exhibit large therapeutic indices are generally preferred.

Protein formulations are generally administered parenterally. When given parenterally, they must be sterile. Sterile diluents include liquids that are pharmaceutically acceptable (safe and non-toxic for administration to a human) and useful for the preparation of a liquid formulation, such as a formulation reconstituted after lyophilization. Exemplary diluents include sterile water, bacteriostatic water for injection (BWFI), a pH buffered solution (e.g., phosphate-buffered saline), sterile saline solution, Ringer's solution or dextrose solution. Diluents can include aqueous solutions of salts and/or buffers.

Excipients are additives that are included in a formulation because they either impart or enhance the stability, delivery and manufacturability of a drug product. Regardless of the reason for their inclusion, excipients are an integral component of a drug product and therefore need to be safe and well tolerated by patients. For protein drugs, the choice of excipients is particularly important because they can affect both efficacy and immunogenicity of the drug. Hence, protein formulations need to be developed with appropriate selection of excipients that afford suitable stability, safety, and marketability.

The excipients described herein are organized either by their chemical type or their functional role in formulations. Brief descriptions of the modes of stabilization are provided when discussing each excipient type. Given the teachings and guidance provided herein, those skilled in the art will readily be able to vary the amount or range of excipient without increasing viscosity to an undesirable level. Excipients may be chosen to achieve a desired osmolality (i.e., isotonic, hypotonic or hypertonic) of the final solution, pH, desired stability, resistance to aggregation or degradation or precipitation, protection under conditions of freezing, lyophilization or high temperatures, or other properties. A variety of types of excipients are known in the art. Exemplary excipients include salts, amino acids, other tonicity agents, surfactants, stabilizers, bulking agents, cryoprotectants, lyoprotectants, anti-oxidants, metal ions, chelating agents and/or preservatives.

Further, where a particular excipient is reported in a formulation by, e.g., percent (%) w/v, those skilled in the art will recognize that the equivalent molar concentration of that excipient is also contemplated.

### Other Stabilizers and Bulking Agents

Stabilizers include a class of compounds that can serve as cryoprotectants, lyoprotectants, and glass forming agents. Cryoprotectants act to stabilize proteins during freezing or in the frozen state at low temperatures. Lyoprotectants stabilize proteins in the freeze-dried solid dosage form by preserving the native-like conformational properties of the protein during dehydration stages of freeze-drying. Glassy state properties have been classified as “strong” or “fragile” depending on their relaxation properties as a function of temperature. It is important that cryoprotectants, lyoprotectants, and glass forming agents remain in the same phase with the protein in order to impart stability. Sugars, polymers, and polyols fall into this category and can sometimes serve all three roles.

Polyols encompass a class of excipients that includes sugars (e.g., mannitol, sucrose, or sorbitol), and other polyhydric alcohols (e.g., glycerol and propylene glycol). The polymer polyethylene glycol (PEG) is included in this category. Polyols are commonly used as stabilizing excipients and/or isotonicity agents in both liquid and lyophilized parenteral protein formulations. Polyols can protect proteins from both physical and chemical degradation pathways.

Exemplary C₃-C₆ polyols include propylene glycol, glycerin (glycerol), threose, threitol, erythrose, erythritol, ribose, arabinose, arabitol, lyxose, maltitol, sorbitol, sorbose, glucose, mannose, mannitol, levulose, dextrose, maltose, trehalose, fructose, xylitol, inositol, galactose, xylose, fructose, sucrose, 1,2,6-hexanetriol and the like. Higher order sugars include dextran, propylene glycol, or polyethylene glycol. Reducing sugars such as fructose, maltose or galactose oxidize more readily than do non-reducing sugars. Additional examples of sugar alcohols are glucitol, maltitol, lactitol or iso-maltulose. Additional exemplary lyoprotectants include glycerin and gelatin, and the sugars mellibiose, melezitose, raffinose, mannotriose and stachyose. Examples of reducing sugars include glucose, maltose, lactose, maltulose, iso-maltulose and lactulose. Examples of non-reducing sugars include non-reducing glycosides of polyhydroxy compounds selected from sugar alcohols and other straight chain polyalcohols. Monoglycosides include compounds obtained by reduction of disaccharides such as lactose, maltose, lactulose and maltulose.

### Amino Acids

In some embodiments, the pharmaceutical compositions described herein further comprise one or more amino acids. Amino acids have found versatile use in protein formulations as buffers, bulking agents, stabilizers and antioxidants. Histidine and glutamic acid are employed to buffer protein formulations in the pH range of 5.5-6.5 and 4.0-5.5 respectively. The imidazole group of histidine has a pka=6.0 and the carboxyl group of glutamic acid side chain has a pKa of 4.3 which makes them suitable for buffering in their respective pH ranges. Glutamic acid is found in some formulations (e.g., Stemgen®). Histidine is commonly found in marketed protein formulations (e.g., Xolair®, Herceptin®, Recombinate®). It provides a good alternative to citrate, a buffer known to sting upon injection. Interestingly, histidine has also been reported to have a stabilizing effect when used at high concentrations in both liquid and lyophilized presentations (Chen B, et al., Pharm Res., 20 (12): 1952-60 (2003)). Histidine (up to 60 mM) was also observed to reduce the viscosity of a high concentration formulation of an antibody. However, in the same study, the authors observed increased aggregation and discoloration in histidine containing formulations during freeze-thaw studies of the antibody in stainless steel containers. The authors attributed this to an effect of iron ions leached from corrosion of steel containers. Another note of caution with histidine is that it undergoes photo-oxidation in the presence of metal ions (Tomita M, et al., Biochemistry, 8 (12): 5149-60 (1969)). The use of methionine as an antioxidant in formulations appears promising; it has been observed to be effective against a number of oxidative stresses (Lam X M, et al., J Pharm Sci., 86 (11): 1250-5 (1997)).

The amino acids glycine, proline, serine and alanine stabilize proteins. Glycine is also a commonly used bulking agent in lyophilized formulations (e.g., Neumega®, Genotropin®, Humatrope®). Arginine has been shown to be an effective agent in inhibiting aggregation and has been used in both liquid and lyophilized formulations (e.g., Activase®, Avonex®, Enbrel® liquid).

### Antioxidants

In some embodiments, the pharmaceutical composition described herein further comprises one or more antioxidants. Oxidation of protein residues arises from a number of different sources. Beyond the addition of specific antioxidants, the prevention of oxidative protein damage involves the careful control of a number of factors throughout the manufacturing process and storage of the product such as atmospheric oxygen, temperature, light exposure, and chemical contamination. The most commonly used pharmaceutical antioxidants are reducing agents, oxygen/free-radical scavengers, or chelating agents. Antioxidants in therapeutic protein formulations must be water-soluble and remain active throughout the product shelf-life. Reducing agents and oxygen/free-radical scavengers work by ablating active oxygen species in solution. Chelating agents such as EDTA can be effective by binding trace metal contaminants that promote free-radical formation. For example, EDTA was utilized in the liquid formulation of acidic fibroblast growth factor to inhibit the metal ion catalyzed oxidation of cysteine residues. EDTA has been used in marketed products like Kineret® and Ontak®.

However, antioxidants themselves can induce other covalent or physical changes to the protein. A number of such cases have been reported in the literature. Reducing agents (like glutathione) can cause disruption of intramolecular disulfide linkages, which can lead to disulfide shuffling. In the presence of transition metal ions, ascorbic acid and EDTA have been shown to promote methionine oxidation in a number of proteins and peptides (Akers M J, and Defelippis M R. Peptides and Proteins as Parenteral Solutions. In: Pharmaceutical Formulation Development of Peptides and Proteins. Sven Frokjaer, Lars Hovgaard, editors. Pharmaceutical Science. Taylor and Francis, UK (1999)); Fransson J. R., J. Pharm. Sci. 86 (9): 4046-1050 (1997); Yin J, et al., Pharm Res., 21 (12): 2377-83 (2004)). Sodium thiosulfate has been reported to reduce the levels of light and temperature induced methionine-oxidation in rhuMab HER2; however, the formation of a thiosulfate-protein adduct was also reported in this study (Lam X M, Yang J Y, et al., J Pharm Sci. 86 (11): 1250-5 (1997)). Selection of an appropriate antioxidant is made according to the specific stresses and sensitivities of the protein.

### Metal Ions

In some embodiments, the pharmaceutical composition further comprises one or more metal ions. In general, transition metal ions are undesired in protein formulations because they can catalyze physical and chemical degradation reactions in proteins. However, specific metal ions are included in formulations when they are co-factors to proteins and in suspension formulations of proteins where they form coordination complexes (e.g., zinc suspension of insulin). Recently, the use of magnesium ions (10-120 mM) has been proposed to inhibit the isomerization of aspartic acid to isoaspartic acid (International Patent Publication No. WO 2004/039337).

Two examples where metal ions confer stability or increased activity in proteins are human deoxyribonuclease (rhDNase, Pulmozyme®), and Factor VIII. In the case of rhDNase, Ca⁺² ions (up to 100 mM) increased the stability of the enzyme through a specific binding site (Chen B, et al., J Pharm Sci., 88 (4): 477-82 (1999)). In fact, removal of calcium ions from the solution with EGTA caused an increase in deamidation and aggregation. However, this effect was observed only with Ca⁺² ions; other divalent cations—Mg⁺², Mn⁺² and Zn⁺² were observed to destabilize rhDNase. Similar effects were observed in Factor VIII. Ca⁺² and Sr⁺² ions stabilized the protein while others like Mg⁺², Mn⁺² and Zn⁺², Cu⁺² and Fe⁺² destabilized the enzyme (Fatouros, A., et al., Int. J. Pharm., 155, 121-131 (1997). In a separate study with Factor VIII, a significant increase in aggregation rate was observed in the presence of Al⁺³ ions (Derrick T S, et al., J. Pharm. Sci., 93 (10): 2549-57 (2004)). The authors note that other excipients like buffer salts are often contaminated with Al⁺³ ions and illustrate the need to use excipients of appropriate quality in formulated products.

### Preservatives

In some embodiments, the pharmaceutical composition further comprises one or more preservatives. Preservatives are necessary when developing multi-use parenteral formulations that involve more than one extraction from the same container. Their primary function is to inhibit microbial growth and ensure product sterility throughout the shelf-life or term of use of the drug product. Commonly used preservatives include phenol, benzyl alcohol, meta-cresol, alkyl parabens such as methyl paraben or propyl paraben, benzalkonium chloride, and benzethonium chloride. Other examples of compounds with antimicrobial preservative activity include octadecyldimethylbenzyl ammonium chloride, hexamethonium chloride. Other types of preservatives include aromatic alcohols such as butyl alcohol, phenol, benzyl alcohol; atechol, resorcinol, cyclohexanol, 3-pentanol. Although preservatives have a long history of use, the development of protein formulations that includes preservatives can be challenging. Preservatives almost always have a destabilizing effect (aggregation) on proteins, and this has become a major factor in limiting their use in multi-dose protein formulations (Roy S, et al., J Pharm Sci., 94 (2): 382-96 (2005)).

Multi-use injection pen presentations include preserved formulations. For example, preserved formulations of hGH are currently available on the market. Norditropin® (liquid, Novo Nordisk), Nutropin AQ® (liquid, Genentech) & Genotropin (lyophilized-dual chamber cartridge, Pharmacia & Upjohn) contain phenol while Somatrope® (Eli Lilly) is formulated with m-cresol.

Several aspects are considered during the formulation development of preserved dosage forms. Optimization of preservative concentration involves testing a given preservative in the dosage form with concentration ranges that confer anti-microbial effectiveness without compromising protein stability. For example, three preservatives were successfully screened in the development of a liquid formulation for interleukin-1 receptor (Type I), using differential scanning calorimetry (DSC). The preservatives were rank ordered based on their impact on stability at concentrations commonly used in marketed products (Remmele R L Jr., et al., Pharm Res., 15 (2): 200-8 (1998)).

Some preservatives can cause injection site reactions, which is another factor for consideration when choosing a preservative. In clinical trials that focused on the evaluation of preservatives and buffers in Norditropin, pain perception was observed to be lower in formulations containing phenol and benzyl alcohol as compared to a formulation containing m-cresol (Kappelgaard A. M., Horm Res. 62 Suppl 3:98-103 (2004)). Interestingly, among the commonly used preservative, benzyl alcohol possesses anesthetic properties (Minogue S C, and Sun D A., Anesth Analg., 100 (3): 683-6 (2005)).

However, the disclosure also contemplates a pharmaceutical composition that does not comprise any preservatives.

### Antigen-Binding Proteins

An “antigen-binding protein” is a protein comprising a portion that binds a specified target antigen (such as CD3 and/or CD38). An antigen-binding protein comprises a scaffold or framework portion that allows the antigen binding portion to adopt a conformation that promotes binding of the antigen-binding protein to the antigen. In exemplary aspects, the antigen-binding protein is an antibody or immunoglobulin, or an antigen-binding antibody fragment, or an antibody protein product.

The term “antibody” refers to an intact antigen-binding immunoglobulin. An “antibody” is a type of an antigen-binding protein. The antibody can be an IgA, IgD, IgE, IgG, or IgM antibody, including any one of IgG1, IgG2, IgG3 or IgG4. In various embodiments, an intact antibody comprises two full-length heavy chains and two full-length light chains. An antibody has a variable region and a constant region. In IgG formats, a variable region is generally about 100-110 or more amino acids, comprises three complementarity determining regions (CDRs), is primarily responsible for antigen recognition, and substantially varies among other antibodies that bind to different antigens. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342:877-883), within a framework region (designated framework regions 1-4, FR1, FR2, FR3, and FR4, by Kabat et al., 1991; see also Chothia and Lesk, 1987, supra). The constant region allows the antibody to recruit cells and molecules of the immune system.

In various aspects, the antibody is a monoclonal antibody. In certain aspects, the antibody is a human antibody. In certain aspects, the antibody (or other antigen-binding protein) is chimeric or humanized. The term “chimeric” refers to an antibody containing domains from two or more different antibodies. A chimeric antibody can, for example, contain the constant domains from one species and the variable domains from a second, or more generally, can contain stretches of amino acid sequence from at least two species. Both “chimeric” and “humanized” often refer to antigen-binding proteins that combine regions from more than one species. A chimeric antibody also can contain domains of two or more different antibodies within the same species. In one embodiment, the chimeric antibody is a CDR grafted antibody.

The term “humanized” when used in relation to antigen-binding proteins refers to antigen-binding proteins (e.g., antibodies) having at least CDR region from a non-human source and which are engineered to have a structure and immunological function more similar to true human antibodies than the original source antibodies. For example, humanizing can involve grafting a CDR from a non-human antibody, such as a mouse antibody, into a human framework region. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., International Patent Publication No. WO 92/11018; Jones, 1986, Nature 321:522-525; and Verhocyen et al., 1988, Science 239:1534-1536, all entirely incorporated by reference. “Back mutation” of selected acceptor framework residues to the corresponding donor residues is often employed to regain affinity that is lost in the initial grafted construct (Sec, e.g., U.S. Pat. Nos. 5,530,101; 5,585,089; 5,693,761; 5,693,762; 6,180,370; 5,859,205; 5,821,337; 6,054,297; and 6,407,213, all entirely incorporated by reference). The humanized antibody optimally also will comprise at least a portion of an immunoglobulin constant region. typically that of a human immunoglobulin, and thus will typically comprise a human Fc region.

Optionally, the antibody of the composition is a bispecific antibody, i.e., an antibody that binds two different targets (e.g., CD3 and a second, different target). In various aspects, the antibody of the composition is a heterodimeric antibody.

In some embodiments, the compositions described herein comprise a heterodimeric antibody comprising a first monomer comprising a first Fc domain and an anti-CD3 scFv. The heterodimeric antibody further comprises a second monomer comprising an anti-CD38 heavy variable domain and a heavy constant domain comprising a second Fc domain. The heterodimeric antibody also comprises light chain comprising a constant domain and an anti-CD38 variable light domain. Features of the monomers are further described below.

An scFv comprises a variable heavy chain, an scFv linker, and a variable light domain. Optionally, the C-terminus of the variable light chain is attached to the N-terminus of the scFv linker, the C-terminus of which is attached to the N-terminus of a variable heavy chain (N-vh-linker-vl-C), although the configuration can be switched (N-vl-linker-vh-C). Thus, specifically included in the depiction and description of scFvs are the scFvs in either orientation. In various aspects, the scFv domain linker is a charged linker. A number of suitable scFv linkers can be used and many are set forth in the Figures. Charged scFv linkers may be employed to facilitate the separation in pI between a first and a second monomer. That is, by incorporating a charged scFv linker, either positive or negative (or both, in the case of scaffolds that use scFvs on different monomers), this allows the monomer comprising the charged linker to alter the pI without making further changes in the Fc domains.

The scFv is covalently attached to the N-terminus of the Fc domain using a domain linker. A “domain linker” links any two domains as outlined herein together. If desired, charged domain linkers can be used. Charged domain linkers can, e.g., increase the pI separation of the monomers of the disclosure as well, and thus those included in the Figures can be used in any embodiment herein where a linker is utilized.

A linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example (GS)n, (GSGGS)n (SEQ ID NO:332), (GGGGS)n (SEQ ID NO:333), and (GGGS)n (SEQ ID NO: 334), where n is an integer of at least one (and generally from 3 to 4), glycine-alanine polymers, alanine-serine polymers, and other flexible linkers. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers.

Other linker sequences may include any sequence of any length of CL/CH1 domain but not all residues of CL/CH1 domain; for example the first 5-12 amino acid residues of the CL/CH1 domains. Linkers can be derived from immunoglobulin light chain, for example Cκ or Cλ. Linkers can be derived from immunoglobulin heavy chains of any isotype, including for example Cγ1, Cγ2, Cγ3, Cγ4, Cα1, Cα2, Cδ, Cε, and Cμ. Linker sequences may also be derived from other proteins such as Ig-like proteins (e.g., TCR, FcR, KIR), hinge region-derived sequences, and other natural sequences from other proteins.

The anti-CD3 scFv comprises (i) a scFv variable light domain comprising vlCDR1 as set forth in SEQ ID NO:15, vlCDR2 as set forth in SEQ ID NO:16, and vlCDR3 as set forth in SEQ ID NO:17, and (ii) a scFv variable heavy domain comprising vhCDR1 as set forth in SEQ ID NO: 11, vhCDR2 as set forth in SEQ ID NO:12, and vhCDR3 as set forth in SEQ ID NO:13. Optionally, the anti-CD3 scFv comprises a variable heavy domain comprising an amino acid sequence at least 90% identical (e.g., 91%, 92%, 93%, 94%, 95%, 96%, 97%, 98%, 99%, or 100% identical) to the amino acid sequence set forth in SEQ ID NO: 10. Also optionally, the anti-CD3 scFv comprises a variable light domain comprising an amino acid sequence at least 90% identical (e.g., 91%, 92%, 93%, 94%, 95%, 96%, 97%, 98%, 99%, or 100% identical) to the amino acid sequence set forth in SEQ ID NO: 14. In this regard, the anti-CD3 scFv, in various embodiments, comprises a variable heavy domain of SEQ ID NO: 10 and a variable light domain of SEQ ID NO: 14. Optionally, the variable heavy and variable light domains are linked by an scFv domain linker comprising the sequence GKPGSGKPGSGKPGSGKPGS (SEQ ID NO: 158). In this regard, the anti-CD3 scFv comprises, in various embodiments, an amino acid sequence at least 90% identical (e.g., 91%, 92%, 93%, 94%, 95%, 96%, 97%, 98%, 99%, or 100% identical) to the amino acid sequence set forth in SEQ ID NO: 18 (scFv of Antibody A). In various aspects, the sequence variation giving rise to less than 100% percent identity to a reference sequence represents modifications outside the CDR sequences. In various aspects, the scFv comprises sequences set forth herein as belonging to Anti-CD3_H1.32_L1.47 (corresponding to Antibody A).

“Fc” or “Fc region” or “Fc domain” refers to the polypeptide comprising the constant region of an antibody excluding the first constant region immunoglobulin domain and in some cases, part of the hinge. Thus, “Fc domain” refers to the last two constant region immunoglobulin domains of IgA, IgD, and IgG, the last three constant region immunoglobulin domains of IgE and IgM, and the flexible hinge N-terminal to these domains. For IgA and IgM. Fc may include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). The heterodimeric antibody is preferably an IgG antibody (which includes several subclasses, including, but not limited to IgG1, IgG2, IgG3, and IgG4). Although the boundaries of the Fc region may vary, the human IgG heavy chain Fc region is usually defined to include residues C226 or P230 to its carboxyl-terminus, wherein the numbering is according to the EU index as in Kabat. In some embodiments, amino acid modifications are made to the Fc region, for example, to alter binding to one or more FcγR receptors or to the FcRn receptor.

In various aspects, the first monomer (i.e., the first Fc domain and the anti-CD3 scFv) comprises an amino acid sequence at least 90% identical to the amino acid sequence set forth in SEQ ID NO:335 (e.g., 91%, 92%, 93%, 94%, 95%, 96%, 97%, 98%, 99%, or 100% identical to the amino acid sequence set forth in SEQ ID NO: 335 (corresponding to Antibody A)).

The heterodimeric antibody optionally further comprises a second monomer comprising i) an anti-CD38 heavy variable domain and ii) a heavy constant domain comprising a second Fc domain. The anti-CD38 heavy variable domain comprises the following CDR sequences: variable heavy (vh) CDR1 as set forth in SEQ ID NO:65, vhCDR2 as set forth in SEQ ID NO:66, and vhCDR3 as set forth in SEQ ID NO:67. Optionally, the anti-CD38 heavy variable domain comprises an amino acid sequence at least 90% identical to the amino acid sequence set forth in SEQ ID NO:64 (e.g., 91%, 92%, 93%, 94%, 95%, 96%, 97%, 98%, 99%, or 100% identical to the amino acid sequence set forth in SEQ ID NO: 64). In various aspects, the second monomer (i.e., the anti-CD38 heavy variable domain and heavy constant domain comprising a second Fc domain) comprises an amino acid sequence at least 90% identical to the amino acid sequence set forth in SEQ ID NO:82 (e.g., 91%, 92%, 93%, 94%, 95%, 96%, 97%, 98%, 99%, or 100% identical to the amino acid sequence set forth in SEQ ID NO: 82 (corresponding to Antibody A)).

In various aspects, the heterodimeric antibody further comprises a light chain comprising a constant domain and an anti-CD38 variable light (vl) domain. The anti-CD38 variable light domain comprises the following CDRs: vlCDR1 as set forth in SEQ ID NO:69, vlCDR2 as set forth in SEQ ID NO:70, and vlCDR3 as set forth in SEQ ID NO:71. Optionally, the anti-CD38 variable light domain comprises an amino acid sequence at least 90% identical to the amino acid sequence set forth in SEQ ID NO:68 (e.g., 91%, 92%, 93%, 94%, 95%, 96%, 97%, 98%, 99%, or 100% identical to the amino acid sequence set forth in SEQ ID NO: 68 (corresponding to Antibody A). In some embodiments, the light chain (comprising the constant domain and the anti-CD38 variable light domain) comprises an amino acid sequence at least 90% identical to the amino acid sequence set forth in SEQ ID NO:84 (e.g., 91%, 92%, 93%, 94%, 95%, 96%, 97%, 98%, 99%, or 100% identical to the amino acid sequence set forth in SEQ ID NO: 84 (corresponding to Antibody A)).

In a preferred embodiment, the heterodimeric antibody is Antibody A and comprises a first monomer comprising an anti-CD3 scFv comprising an anti-CD3 variable light domain comprising the amino acid sequence of SEQ ID NO: 14 and an anti-CD3 variable heavy domain comprising the amino acid sequence of SEQ ID NO: 10, a second monomer comprising an anti-CD38 variable heavy domain comprising the amino acid sequence of SEQ ID NO: 64, and a light chain comprising a variable light domain comprising the amino acid sequence of SEQ ID NO: 68. For example, in one embodiment, the heterodimeric antibody comprises a first monomer comprising the amino acid sequence of SEQ ID NO: 335, a second monomer comprising the amino acid sequence of SEQ ID NO: 82, and a light chain comprising the amino acid sequence of SEQ ID NO: 84.

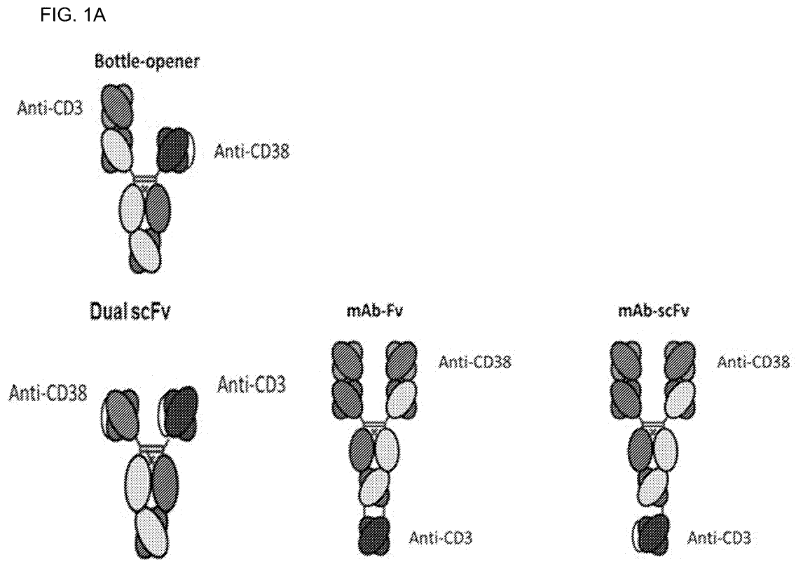

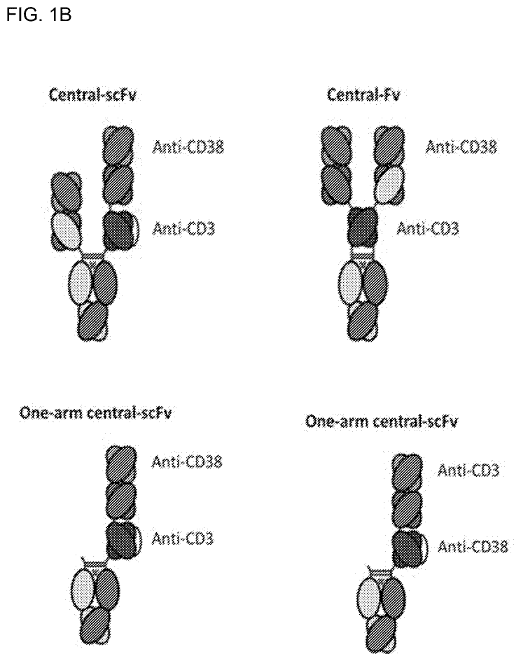

In some embodiments, the compositions described herein comprise a heterodimeric antibody which binds CD3 and STEAP1. STEAP1 is a 339 amino acid protein comprising six transmembrane domains, resulting in three extracellular loops and two intracellular loops. The amino acid sequence of human STEAP1 is set forth herein as SEQ ID NO: 356. The estimated positions of the extracellular loops are amino acids 92-118 (extracellular loop 1), amino acids 185-217 (extracellular loop 2), and amino acids 279-290 (extracellular loop 3). STEAP1 is differentially expressed in prostate cancer compared to normal tissues, and increased expression in bone and lymph node prostate cancer metastatic lesions was observed compared to primary prostate cancer samples. STEAP1 represents an ideal target for diagnostics and antibody-based therapeutics, such as a bispecific anti-STEAP1/anti-CD3 T cell recruiting antibody to, e.g., trigger T cell dependent cellular cytotoxicity or redirected lysis of prostate cancer cells. The antigen-binding protein of the disclosure optionally binds STEAP1 in a region outside of the second extracellular loop. The antigen-binding protein, in at least one embodiment, binds a region of STEAP1 within amino acids 92-118 (extracellular loop 1) and/or amino acids 279-290 (extracellular loop 3). Also optionally, the antigen-binding protein does not bind STEAP2 (UniProtKB No. Q8NFT2; SEQ ID NO: 177). The disclosure provides a composition comprising an antigen-binding protein that binds STEAP1 and CD3 in any of the formats described herein, optionally in “bottle opener” in FIG. 1A or the Central-scFv format (also referred to as the “XmAb²⁺¹” format) in FIG. 1B.

In some embodiments, the compositions described herein comprise a heterodimeric antibody comprising a first monomer comprising a first Fc domain and an anti-CD3 scFv and further comprising a second monomer comprising an anti-STEAP1 heavy variable domain and a heavy constant domain comprising a second Fc domain. The heterodimeric antibody also comprises light chain comprising a constant domain and an anti-STEAP1 variable light domain. Exemplary aspects of monomers that constitute the scaffold are described above. In this embodiment, the heterodimeric antibody further comprises a second monomer comprising i) an anti-STEAP1 heavy variable domain and ii) a heavy constant domain comprising a second Fc domain. The anti-STEAP1 heavy variable domain optionally comprises variable heavy (vh) CDR1 as set forth in SEQ ID NO:360, vhCDR2 as set forth in SEQ ID NO:361 or SEQ ID NO: 363, and vhCDR3 as set forth in SEQ ID NO: 362. The anti-STEAP1 variable light domain of the light chain comprises vlCDR1 as set forth in SEQ ID NO: 357, vlCDR2 as set forth in SEQ ID NO: 358 and vlCDR3 as set forth in SEQ ID NO: 359. Alternatively, the anti-STEAP1 variable heavy domain comprises vhCDR1 as set forth in SEQ ID NO: 368, vhCDR2 as set forth in SEQ ID NO: 369, and vhCDR3 as set forth in SEQ ID NO: 370; and the variable light domain comprises vlCDR1 as set forth in SEQ ID NO: 371, vlCDR2 as set forth in SEQ ID NO: 372, and vlCDR3 as set forth in SEQ ID NO: 373.

Optionally, the anti-STEAP1 heavy variable domain comprises an amino acid sequence at least 90% identical to the amino acid sequence set forth in SEQ ID NO: 377 or SEQ ID NO: 379 (e.g., 91%, 92%, 93%, 94%, 95%, 96%, 97%, 98%, 99%, or 100% identical to the amino acid sequence set forth in SEQ ID NO: 377 or SEQ ID NO: 379). Optionally, the anti-STEAP1 variable light domain comprises an amino acid sequence at least 90% identical to the amino acid sequence set forth in SEQ ID NO: 378 (e.g., 91%, 92%, 93%, 94%, 95%, 96%, 97%, 98%, 99%, or 100% identical to the amino acid sequence set forth in SEQ ID NO: 378). In preferred embodiments, the anti-STEAP1 variable heavy domain comprises SEQ ID NO: 380 or SEQ ID NO: 379 and the anti-STEAP1 variable light domain comprises SEQ ID NO: 378.

Also optionally, the anti-STEAP1 heavy variable domain comprises an amino acid sequence at least 90% identical to the amino acid sequence set forth in SEQ ID NO: 380 (e.g., 91%, 92%, 93%, 94%, 95%, 96%, 97%, 98%, 99%, or 100% identical to the amino acid sequence set forth in SEQ ID NO: 380). Optionally, the anti-STEAP1 variable light domain comprises an amino acid sequence at least 90% identical to the amino acid sequence set forth in SEQ ID NO: 381 (e.g., 91%, 92%, 93%, 94%, 95%, 96%, 97%, 98%, 99%, or 100% identical to the amino acid sequence set forth in SEQ ID NO: 381). In preferred embodiments, the anti-STEAP1 variable heavy domain comprises SEQ ID NO: 380 and the anti-STEAP1variable light domain comprises SEQ ID NO: 381.

In some embodiments, such as embodiments wherein the antigen-binding protein is a heterodimeric antibody that binds CD3 and STEAP1, and the CD3 binding domain (optionally an scFv as discussed above) comprises a variable heavy domain comprising heavy chain CDRs comprising vhCDR1 set forth in SEQ ID NO: 383, vhCDR2 set forth in SEQ ID NO: 384, and vhCDR3 set forth in SEQ ID NO: 385, and a variable light domain comprising light chain CDRs comprising vlCDR1 set forth in SEQ ID NO: 387, vlCDR2 set forth in SEQ ID NO: 388, and vlCDR3 set forth in SEQ ID NO: 389. For example, the disclosure provides compositions comprising a multispecific (e.g., bispecific) construct comprising an anti-CD3 variable heavy domain comprising an amino acid sequence at least 90% identical (e.g., 91%, 92%, 93%, 94%, 95%, 96%, 97%, 98%, 99%, or 100% identical) to SEQ ID NO:382 and/or an anti-CD3 variable light domain comprising an amino acid sequence at least 90% identical (e.g., 91%, 92%, 93%, 94%, 95%, 96%, 97%, 98%, 99%, or 100% identical) to SEQ ID NO:386. In various aspects, the heterodimeric antibody comprises an anti-CD3 scFv comprising SEQ ID NO: 390. scFvs are described in more detail above, and features of the scFv described above also apply here.

In various embodiments, the antigen-binding protein is a heterodimeric antibody in the Central-scFv or “XmAb²⁺¹” format shown in FIG. 1B. The format relies on the use of an inserted scFv domain forming a third antigen-binding domain, wherein the Fab portions of the two monomers bind one target and the “extra” scFv domain binds another. The scFv domain is inserted between the Fc domain and the CH1-Fv region of one of the monomers, thus providing the third antigen-binding domain. In this embodiment, one monomer comprises a first heavy chain comprising a first variable heavy domain, a CH1 domain (and optional linker/hinge) and Fc domain, with a scFv comprising a scFv variable light domain, an scFv linker and a scFv variable heavy domain. The scFv is covalently attached between the C-terminus of the CH1 domain of the heavy constant domain and the N-terminus of the first Fc domain using optional domain linkers (VHl-CHl-[optional domain linker]-VH2-scFv linker-VL2-[optional domain linker including the hinge]-CH2-CH3, or the opposite orientation for the scFv, VH1-CH1-[optional domain linker]-VL2-scFv linker-VH2-[optional domain linker including the hinge]-CH2-CH3). In some embodiments, the first monomer is VH1-CH1-domain linker-VH2-scFv linker-VL2-domain linker-CH2-CH3. The other monomer is a standard Fab side (i.e., VH1-CH1-domain linker (e.g., hinge)-CH2-CH3). This embodiment further utilizes a common light chain comprising a variable light domain and a constant light domain, which associates with the heavy chains to form two identical Fabs that bind a target. As for many of the embodiments herein, these constructs include skew variants, pI variants, ablation variants, additional Fc variants, etc. as desired and described herein and in in International Patent Publication No. WO 2017/21870.

In some aspects, the antigen-binding protein is a heterodimeric antibody in the “XmAb²⁺¹” format that binds CD3 and STEAP1, and the CD3 binding domain (optionally an scFv as discussed above) comprises a variable heavy domain comprising heavy chain CDRs comprising vhCDR1 set forth in SEQ ID NO: 383, vhCDR2 set forth in SEQ ID NO: 384, and vhCDR3 set forth in SEQ ID NO: 385, and a variable light domain comprising light chain CDRs comprising vlCDR1 set forth in SEQ ID NO: 387, vlCDR2 set forth in SEQ ID NO:388 and vlCDR3 set forth in SEQ ID NO: 389. For example, the construct optionally comprises an anti-CD3 variable heavy domain comprising an amino acid sequence at least 90% identical (e.g., 91%, 92%, 93%, 94%, 95%, 96%, 97%, 98%, 99%, or 100% identical) to SEQ ID NO:380 and/or an anti-CD3 variable light domain comprising an amino acid sequence at least 90% identical (e.g., 91%, 92%, 93%, 94%, 95%, 96%, 97%, 98%, 99%, or 100% identical) to SEQ ID NO: 381. In various aspects, the scFv linker comprises the amino acid sequence of SEQ ID NO: 391. In various aspects, the heterodimeric antibody comprises an anti-CD3 scFv comprising SEQ ID NO: 390.

In various aspects, the antigen-binding protein is an XmAb²⁺¹ format heterodimeric antibody comprising two Fabs that bind STEAP1. In this regard, in some embodiments, the first variable heavy domain and the second variable heavy domain of the heterodimeric antibody comprise vhCDR1 set forth in SEQ ID NO: 360, vhCDR2 set forth in SEQ ID NO: 361 or SEQ ID NO: 363, and vhCDR3 set forth in SEQ ID NO: 362; and the variable light domain comprises vlCDR1 set forth in SEQ ID NO: 357, vlCDR2 set forth in SEQ ID NO: 358, and vlCDR3 set forth in SEQ ID NO: 359. Alternatively, the first variable heavy domain and the second variable heavy domain comprise vhCDR1 set forth in SEQ ID NO: 368, vhCDR2 set forth in SEQ ID NO: 369, and vhCDR3 set forth in SEQ ID NO: 370; and the variable light domain comprises vlCDR1 set forth in SEQ ID NO: 371, vlCDR2 set forth in SEQ ID NO: 372, and vlCDR3 set forth in SEQ ID NO: 373. In preferred embodiments, the first variable heavy domain and the second variable heavy domain comprise an amino acid sequence at least 90% identical (e.g., 91%, 92%, 93%, 94%, 95%, 96%, 97%, 98%, 99%, or 100% identical) to SEQ ID NO: 377 (corresponding to Antibody B) or SEQ ID NO: 379 and/or the variable light domain comprises an amino acid sequence at least 90% identical (e.g., 91%, 92%, 93%, 94%, 95%, 96%, 97%, 98%, 99%, or 100% identical) to SEQ ID NO: 378 (corresponding to Antibody B). Alternatively, the first variable heavy domain and the second variable heavy domain comprise an amino acid sequence at least 90% identical (e.g., 91%, 92%, 93%, 94%, 95%, 96%, 97%, 98%, 99%, or 100% identical) to SEQ ID NO: 380 and/or the variable light domain comprises an amino acid sequence at least 90% identical (e.g., 91%, 92%, 93%, 94%, 95%, 96%, 97%, 98%, 99%, or 100% identical) to SEQ ID NO: 381. As described above, variation in any variable domain sequence (or full length monomer sequence) described herein which results in less than 100% sequence identity compared to a reference sequence preferably occurs outside the CDR regions.

Thus, the disclosure provides a pharmaceutical composition comprising a heterodimeric antibody, which comprises (a) a first monomer comprising a first heavy chain comprising 1) a first variable heavy domain; 2) a first constant heavy chain comprising a first CH1 domain and a first Fc domain; and 3) a scFv that binds human CD3. The scFv comprises (i) a scFv variable light domain comprising vlCDR1 set forth in SEQ ID NO:387, vlCDR2 set forth in SEQ ID NO: 388, and vlCDR3 set forth in SEQ ID NO: 389, (ii) an scFv linker, and (iii) a scFv variable heavy domain comprising vhCDR1 set forth in SEQ ID NO: 383, vhCDR2 set forth in SEQ ID NO: 384, and vhCDR3 set forth in SEQ ID NO: 385. The scFv is covalently attached between the C-terminus of said CH1 domain and the N-terminus of said first Fc domain using domain linker(s). The heterodimeric antibody further comprises b) a second monomer comprising a second heavy chain comprising a second variable heavy domain and a second constant heavy chain comprising a second Fc domain and c) a common light chain comprising a variable light domain and a constant light domain; wherein the first variable heavy domain and the variable light domain bind human STEAP1, and the second variable heavy domain and the variable light domain bind human STEAP1. In some aspects, the first variable heavy domain and the second variable heavy domain comprises heavy chain CDRs comprising vhCDR1 set forth in SEQ ID NO: 360, vhCDR2 set forth in SEQ ID NO: 361 or SEQ ID NO: 363, and vhCDR3 set forth in SEQ ID NO: 362. The variable light domain optionally comprises light chain CDRs comprising vlCDR1 set forth in SEQ ID NO: 357, vlCDR2 set forth in SEQ ID NO: 358, and vlCDR3 set forth in SEQ ID NO: 359. Alternatively, the first variable heavy domain and the second variable heavy domain comprise heavy chain CDRs comprising vhCDR1 set forth in SEQ ID NO: 368, vhCDR2 set forth in SEQ ID NO: 369, and vhCDR3 set forth in SEQ ID NO: 370. The variable light domain optionally comprises light chain CDRs comprising vlCDR1 set forth in SEQ ID NO: 371, vlCDR2 set forth in SEQ ID NO: 372, and vlCDR3 set forth in SEQ ID NO: 373. Optionally, the first variable heavy domain and the second variable heavy domain comprise an amino acid sequence at least 90% identical (e.g., 91%, 92%, 93%, 94%, 95%, 96%, 97%, 98%, 99%, or 100% identical) to SEQ ID NO: 377 (corresponding to Antibody B) or 379 and/or the variable light domain comprises an amino acid sequence at least 90% identical (e.g., 91%, 92%, 93%, 94%, 95%, 96%, 97%, 98%, 99%, or 100% identical) to SEQ ID NO: 378 (corresponding to Antibody B). The scFv optionally comprises a variable heavy region and a variable light region of having amino acid sequences at least 90% identical (e.g., 91%, 92%, 93%, 94%, 95%, 96%, 97%, 98%, 99%, or 100% identical) to SEQ ID NO: 382 and SEQ ID NO: 386, respectively, and the scFv linker optionally comprises SEQ ID NO: 391. The scFv comprises the sequence of SEQ ID NO: 390, in various embodiments.

In various aspects of the disclosure, the anti-CD3/anti-STEAP1 antigen-binding protein is an XmAb²⁺¹ format heterodimeric antibody comprising a first monomer comprising an amino acid sequence at least 90% identical (e.g., 91%, 92%, 93%, 94%, 95%, 96%, 97%, 98%, 99%, or 100% identical) to SEQ ID NO: 366 or 367, a second monomer comprising an amino acid sequence at least 90% identical (e.g., 91%, 92%, 93%, 94%, 95%, 96%, 97%, 98%, 99%, or 100% identical) to SEQ ID NO: 365, and a common light chain comprising an amino acid sequence of at least 90% identical (e.g., 91%, 92%, 93%, 94%, 95%, 96%, 97%, 98%, 99%, or 100% identical) to SEQ ID NO: 364. In some embodiments, the anti-CD3/anti-STEAP1 antigen-binding protein is an XmAb²⁺¹ format heterodimeric antibody comprising a first monomer comprising an amino acid sequence at least 90% identical (e.g., 91%, 92%, 93%, 94%, 95%, 96%, 97%, 98%, 99%, or 100% identical) to SEQ ID NO: 366, a second monomer comprising an amino acid sequence at least 90% identical (e.g., 91%, 92%, 93%, 94%, 95%, 96%, 97%, 98%, 99%, or 100% identical) to SEQ ID NO: 365, and a common light chain comprising an amino acid sequence at least 90% identical (e.g., 91%, 92%, 93%, 94%, 95%, 96%, 97%, 98%, 99%, or 100% identical) to SEQ ID NO: 364 (corresponding to Antibody B). Alternatively, the anti-CD3/anti-STEAP1 antigen-binding protein is an XmAb²⁺¹ format heterodimeric antibody comprising a first monomer comprising an amino acid sequence at least 90% identical (e.g., 91%, 92%, 93%, 94%, 95%, 96%, 97%, 98%, 99%, or 100% identical) to SEQ ID NO: 376, a second monomer comprising an amino acid sequence at least 90% identical (e.g., 91%, 92%, 93%, 94%, 95%, 96%, 97%, 98%, 99%, or 100% identical) to SEQ ID NO: 375, and a common light chain comprising an amino acid sequence at least 90% identical (e.g., 91%, 92%, 93%, 94%, 95%, 96%, 97%, 98%, 99%, or 100% identical) to SEQ ID NO: 374.

In various aspects, the antigen-binding protein comprises a first heavy chain comprising VHl-CHl-[domain linker]-VH2-scFv linker-VL2-[domain linker (optionally including the hinge)]-CH2-CH3; a second heavy chain comprising a VH1-CH1-domain linker-CH2-CH3; and a common light chain comprising a VL1; wherein VH1 and VL1 bind STEAP 1 and VH2 and VL2 bind CD3. In this format, VH2 optionally comprises CDR sequences of SEQ ID NO: 383 (CDR1), SEQ ID NO: 384 (CDR2), and SEQ ID NO: 385 (CDR3), while VL2 comprises CDR sequences of SEQ ID NO: 387 (CDR1), SEQ ID NO: 388 (CDR2), and SEQ ID NO: 389 (CDR3). VH1 comprises CDR sequences of SEQ ID NO: 360 (CDR1), SEQ ID NO: 361 or 363 (CDR2), and SEQ ID NO: 362 (CDR3); and VL1 comprises CDR sequences of SEQ ID NO: 357 (CDR1), SEQ ID NO: 358 (CDR2), and SEQ ID NO: 359 (CDR3). Alternatively, VH1 comprises CDR sequences of SEQ ID NO: 368 (CDR1), SEQ ID NO: 369 (CDR2), and SEQ ID NO: 370 (CDR3); and VL1 comprises CDR sequences of SEQ ID NO: 371 (CDR1), SEQ ID NO: 372 (CDR2), and SEQ ID NO: 373 (CDR3). Optionally, the antigen-binding protein comprises modifications in the first heavy chain including, but not limited to, E233P, delL234, L235V, G236A, S267K, r292c, n297g, v302c, E357Q, and S364K (EU numbering, lower case letters referencing SEFL2 substitutions described further herein), and the second heavy chain comprises modifications including, but not limited to, N208D, E233P, delL234, L235V, G236A, S267K, r292c Q295E, n297g. v302c, L368D, K370S, N384D, Q418E, and N421D (EU numbering, lower case letters referencing SEFL2 substitutions described further herein). A linker for use in the context of this embodiment is optionally GKPGSGKPGSGKPGSGKPGS (SEQ ID NO: 391).

The Fc domains of the central scFv format optionally comprise skew variants (e.g., selected from the group consisting of S364K/E357Q: L368D/K370S; L368D/K370S: S364K; L368E/K370S: S364K; T411T/E360E/Q362E: D401K; L368D/K370S: S364K/E357L, K370S: S364K/E357Q. T366S/L368A/Y407V: T366W and T366S/L368A/Y407V/Y349C: T366W/S354C), optionally ablation variants, optionally charged scFv linkers, and the heavy chain comprises pI variants. In some embodiments, the central scFv format includes skew variants, pI variants, and ablation variants. Accordingly, some embodiments include formats that comprise: a) a first monomer that comprises the skew variants S364K/E357Q, the ablation variants E233P/L234V/L235A/G236del/S267K, and a first variable heavy domain that, with the first variable light domain of the light chain, makes up an Fv that binds to a first target, and a second variable heavy domain; b) a second monomer that comprises the skew variants L368D/K370S, the pI variants N208D/Q295E/N384D/Q418E/N421D, the ablation variants E233P/L234V/L235A/G236del/S267K, and a first variable heavy domain that, with the first variable light domain, makes up the Fv that binds to the first target, and a second variable light chain, that together with the second variable heavy chain forms an Fv that binds a second target; and c) a light chain comprising a first variable light domain and a constant light domain.

The different binding regions of a multispecific antigen-binding protein independently display a KD for their respective antigen (e.g., CD3 and STEAP1 or CD3 and CD38) of less than or equal to 10⁻⁴ M, less than or equal to 10⁻⁵ M, less than or equal to 10⁻⁶ M, less than or equal to 10⁻⁷ M, less than or equal to 10⁻⁸ M, less than or equal to 10⁻⁹ M, less than or equal to 10⁻¹⁰ M. less than or equal to 10⁻¹¹ M, or less than or equal to 10⁻¹² M, or less than or equal to 10⁻¹³ M (e.g., 10⁻⁷ M to 10⁻¹² M), where KD refers to a dissociation rate of a particular antibody-antigen interaction. The STEAP1 binding region (or CD38 binding region) need not bind STEAP1 (or CD38) with the same affinity as, e.g., the CD3 binding region binds CD3.

In some embodiments, the formulation comprises an antigen-binding protein (e.g., antibody) described herein in an amount ranging from about 50 μg to about 200 mg (or from about 500 μg to about 150 mg, or from about 50 mg to about 200 mg, or about 50 mg to about 150 mg, or about 50 mg to about 100 mg, or about 50 mg to about 75 mg). In some embodiments, the formulation comprises an antibody in an amount of about 50 μg, about 100 μg, about 150 μg. about 200 μg, about 250 μg, about 300 μg, about 350 μg, about 400 μg. about 450 μg, about 500 μg, about 550 μg, about 600 μg, about 650 μg, about 700 μg, about 750 μg, about 800 μg, about 850 μg, about 900 μg, about 950 μg, about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95, mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg or about 200 mg.

In some embodiments, the formulation comprises an antigen-binding protein (e.g., antibody) in a concentration ranging from about 0.1 to about 20 mg/mL (or from about 0.5 to about 10 mg/mL, or from about 1 to about 10 mg/mL or from about 1 to about 20 mg/mL, or from about 10 to about 20 mg/mL). In some embodiments, the formulation comprises an antigen-binding protein (e.g., antibody, such as any of the heterodimeric antibodies described herein) in a concentration of about 0.1 mg/mL, about 0.5 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL.

In some embodiments, the formulation comprises an antigen-binding protein (e.g., antibody) in a concentration ranging from about 0.1 to about 8 mg/mL (or from about 0.5 to about 5 mg/mL or from about 1 to about 5 mg/mL, or from about 3 to about 6 mg/mL). In some embodiments, the formulation comprises an antigen-binding protein (e.g., antibody, such as any of the heterodimeric antibodies described herein) in a concentration of about 0.1 mg/mL, about 0.5 mg/mL, about 1 mg/mL, about 2 mg/mL, about 3 mg/mL, about 4 mg/mL, about 5 mg/mL, about 6 mg/mL, about 7 mg/mL, or about 8 mg/mL.

Heterodimeric antibody formats are further described in International Patent Publication No. WO 2017/218707, incorporated by reference herein in its entirety and particularly with respect to the figures and figure legends. In a preferred aspect, the heterodimeric antibody adopts the structure termed “bottle opener” in FIG. 1A. One heavy chain of the “bottle opener” format contains the scFv and the other heavy chain is a “regular” Fab format, comprising a traditional heavy chain and a light chain. The two heavy chains are brought together by the use of amino acid variants in the constant regions (e.g., the Fc domain, the CH1 domain and/or the hinge region) that promote the formation of heterodimeric antibodies. There are several distinct advantages to the “bottle opener” format. Antibody analogs relying on two scFv constructs often have stability and aggregation problems, which is alleviated in the present disclosure by the addition of a “regular” heavy and light chain pairing. In addition, as opposed to formats that rely on two heavy chains and two light chains, there is no issue with the incorrect pairing of heavy and light chains (e.g., heavy 1 pairing with light 2, etc.).

The heterodimeric antibody includes, in various aspects, modifications as compared wild-type antibody domain sequences to promote heterodimeric antibody formation (i.e., reduce homodimerization), adjust antibody functionality, etc. Modifications generally are focused in the Fc domain (although this is not required). Modifications are referenced by the amino acid position of the substitution, deletion, or insertion with respect to the native sequence. For example, N434S or 434S is an Fc domain substitution of serine at position 434 relative to the parent Fc polypeptide, wherein the numbering is according to the EU index. Likewise, M428L/N434S defines an Fc modification having substitutions M428L and N434S relative to the parent Fc polypeptide. The identity of the wild-type amino acid may be unspecified, in which case the aforementioned variant is referred to as 428L/434S. The order in which substitutions are provided is arbitrary, that is to say that, for example, 428L/434S is the same as M428L/N434S, and so on. For all positions discussed that relate to antibodies, unless otherwise noted, amino acid position numbering is according to the EU index. The EU index or EU index as in Kabat or EU numbering scheme refers to the numbering of the EU antibody (Edelman et al., 1969, Proc Natl Acad Sci USA 63:78-85, hereby entirely incorporated by reference). The modification can be an addition, deletion, or substitution. Substitutions can include naturally occurring amino acids and, in some cases, synthetic amino acids. Examples include U.S. Pat. No. 6,586,207; U.S. Patent Publication No. 2004-0214988A1; International Patent Publication Nos. WO 98/48032; WO 03/073238; WO 05/35727A2; WO 05/74524A2; WO 17/218707; J. W. Chin et al., (2002), Journal of the American Chemical Society 124:9026-9027; J. W. Chin, & P. G. Schultz, (2002), ChemBioChem 11:1135-1137; J. W. Chin, et al., (2002), PICAS United States of America 99:11020-11024; and, L. Wang, & P. G. Schultz, (2002), Chem. 1-10, all entirely incorporated by reference.

There are a number of mechanisms that can be used to generate the heterodimeric protein. Amino acid variants that lead to the production of heterodimers are referred to as “heterodimerization variants.” Heterodimerization variants can include steric variants (e.g., the “knobs and holes” or “skew” variants described below and the “charge pairs” variants described below) as well as “pI variants,” which allow purification of homodimers away from heterodimers. As is generally described in International Patent Publication No. WO 2014/145806 and WO 2017/218707, hereby incorporated by reference in their entirety and specifically for the discussion of “heterodimerization variants,” useful mechanisms for heterodimerization include “knobs and holes” (“KIH”; sometimes herein as “skew” variants), “electrostatic steering” or “charge pairs” as described in WO2014/145806, pI variants as described in WO2014/145806, and general additional Fc variants as outlined in WO2014/145806 and herein.

There are several basic mechanisms that can lead to ease of purifying heterodimeric antibodies; one relies on the use of pI variants, such that each monomer has a different pI, thus allowing the isoelectric purification of A-A, A-B and B-B dimeric proteins. Alternatively, some scaffold formats, such as the “bottle opener” format, also allows separation on the basis of size. It is also possible to “skew” the formation of heterodimers over homodimers. Thus, a combination of steric heterodimerization variants and pI or charge pair variants find particular use in the invention.

### A. pI (Isoelectric Point) Variants

For pI variants, amino acid modifications can be introduced into one or both of the monomer polypeptides; that is, the pI of one of the monomers (referred to herein for simplicity as “monomer A”) can be engineered away from monomer B, or both monomer A and B can be changed, with the pI of monomer A increasing and the pI of monomer B decreasing. The pI changes of either or both monomers can be done by removing or adding a charged residue (e.g., a neutral amino acid is replaced by a positively or negatively charged amino acid residue, e.g., glycine to glutamic acid), changing a charged residue from positive or negative to the opposite charge (aspartic acid to lysine) or changing a charged residue to a neutral residue (e.g., loss of a charge; lysine to serine). A number of these variants are shown in the Figures. These modifications create a sufficient change in pI in at least one of the monomers such that heterodimers can be separated from homodimers. As will be appreciated by those in the art, this can be achieved by using a “wild type” heavy chain constant region and a variant region that has been engineered to either increase or decrease it's pI (wt A−+B or wt A−−B), or by increasing one region and decreasing the other region (A+−B− or A−B+).

Thus, in various aspects, the heterodimeric antibody comprises one or more modifications in the constant region(s) to alter the isoelectric point (pI) of at least one, if not both, of the monomers of a heterodimeric protein to form “pI antibodies” by incorporating amino acid substitutions (“pI variants” or “pI substitutions”) into one or both of the monomers. The separation of the heterodimers from the two homodimers can be accomplished if the pIs of the two monomers differ by as little as 0.1 pH unit, with 0.2, 0.3, 0.4 and 0.5 or greater all being suitable.

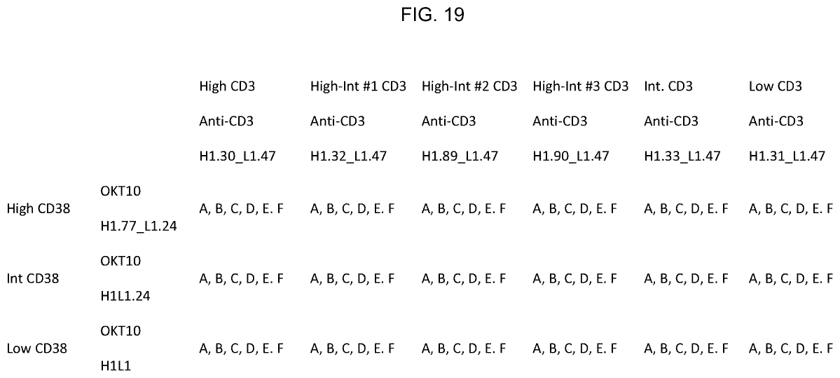

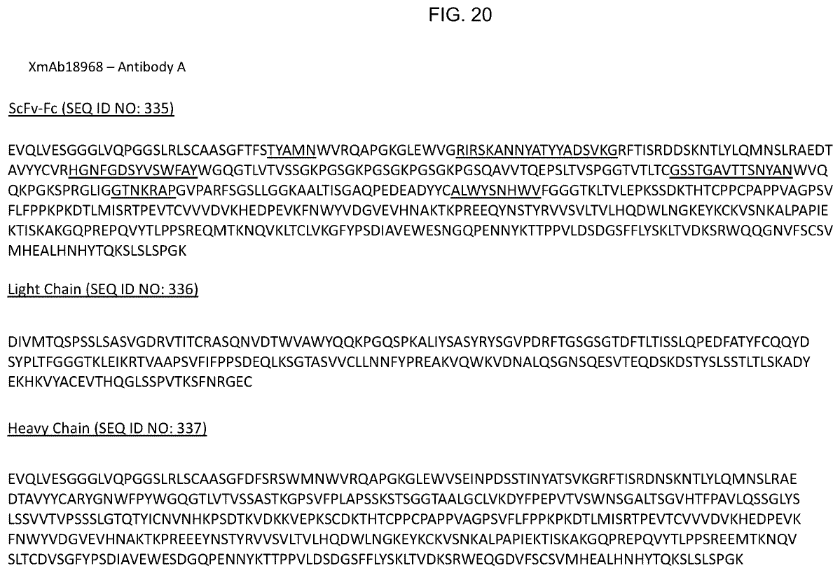

The number of pI variants to be included on each or both monomer(s) to achieve good separation will depend in part on the starting pI of the components, for example, the starting pI of the anti-CD3 scFv and anti-CD38 Fab. That is, to determine which monomer to engineer or in which “direction” (e.g. more positive or more negative), the Fv sequences of the two domains are calculated and a decision is made from there. Different Fvs will have different starting pIs which can be exploited. In some embodiments, the change in pI is calculated on the basis of the variant heavy chain constant domain, using the chart in the FIG. 19 of U.S. Patent Publication No. 2014/0370013. Alternatively, the pI of each monomer can be compared. In general, the pIs are engineered to result in a total pI difference of each monomer of at least about 0.1 logs, with 0.2 to 0.5 being preferred.

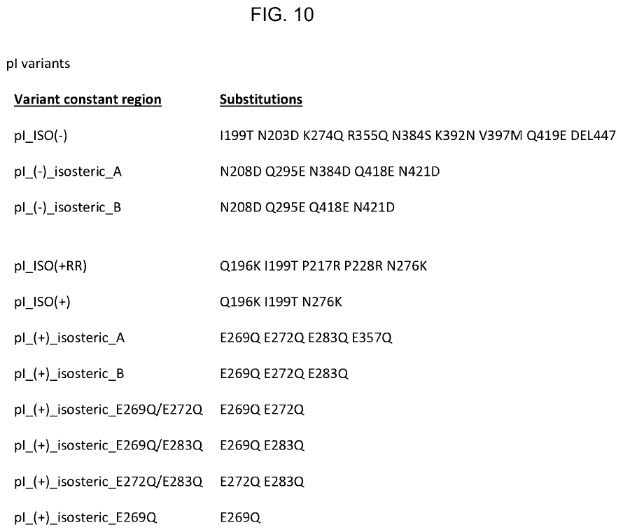

Preferred combinations of pI variants are shown in FIG. 10. These changes are shown relative to IgG1, but all isotypes can be altered this way, as well as isotype hybrids. In the case where the heavy chain constant domain is from IgG2-4, R133E and R133Q can also be used.

In one embodiment, the Fab monomer (the negative side) comprises the substitutions 208D/295E/384D/418E/421D (N208D/Q295E/N384D/Q418E/N421D (relative to human IgG1) and the scFv monomer (the positive side) comprises a positively charged scFv linker, including (GKPGS)₄.

Modifications to adjust pI also can be made in the light chain. Amino acid substitutions for lowering the pI of the light chain include, but are not limited to, K126E, K126Q. K145E, K145Q, N152D, S156E, K169E, S202E, K207E and adding peptide DEDE at the C-terminus of the light chain. Changes in this category based on the constant lambda light chain include one or more substitutions at R108Q. Q124E, K126Q. N138D, K145T and Q199E. In addition, increasing the pI of the light chains can also be done.

### B. Skew/Steric Variants

There are a number of suitable pairs of sets of heterodimerization skew variants. These variants come in “pairs” of “sets.” That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other; that is, these pairs of sets form an interface between the two monomers that encourages heterodimer formation and discourages homodimer formation, allowing the percentage of heterodimers that spontaneously form under biological conditions to be over 90%, rather than the expected 50% (25% homodimer A/A: 50% heterodimer A/B: 25% homodimer B/B).

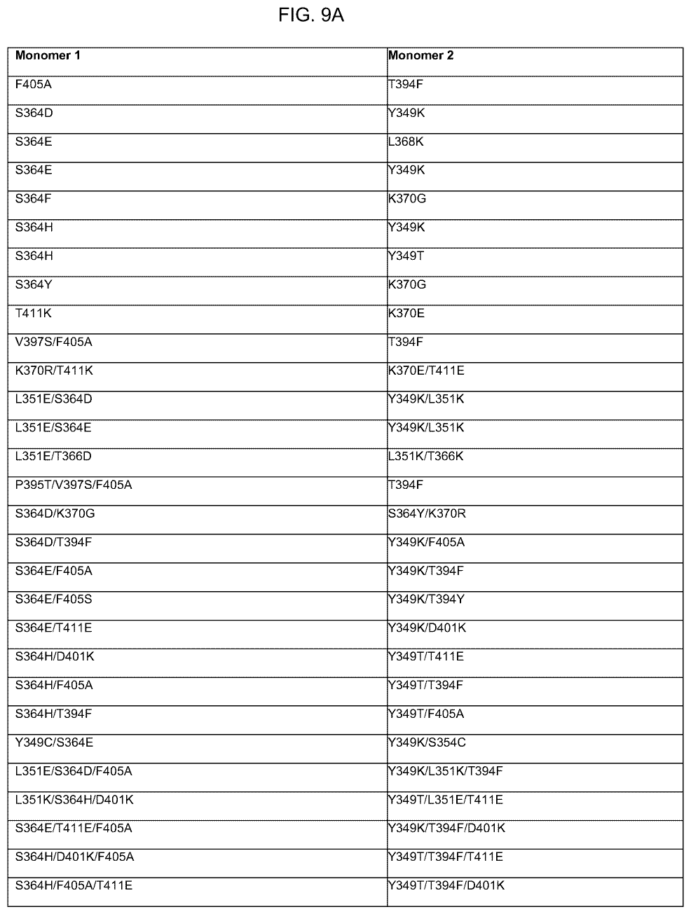

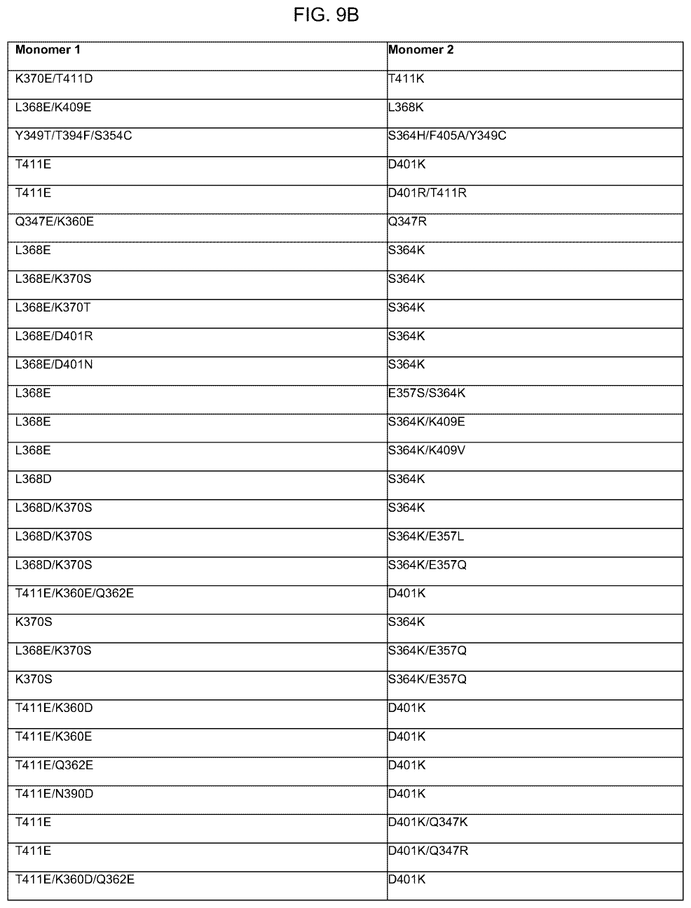

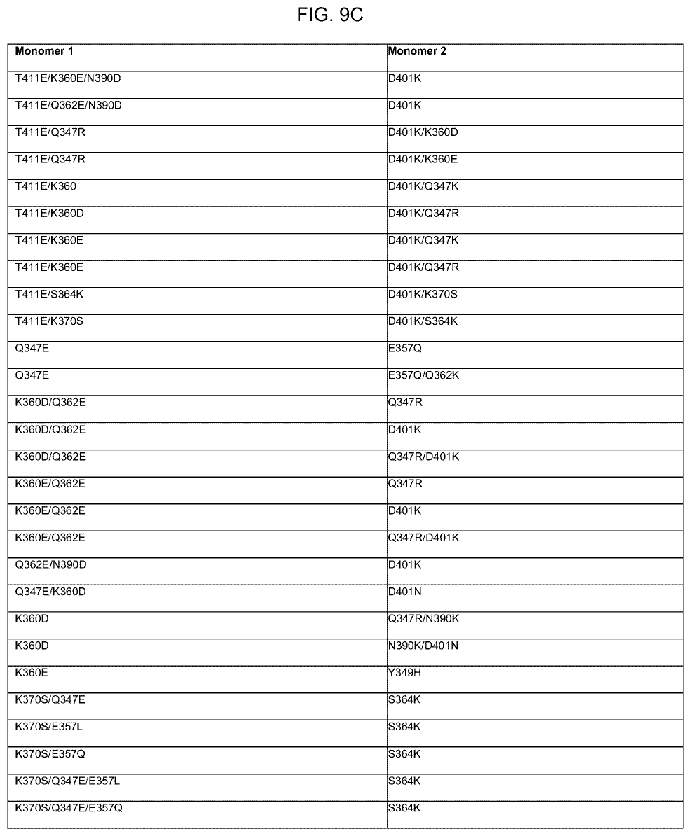

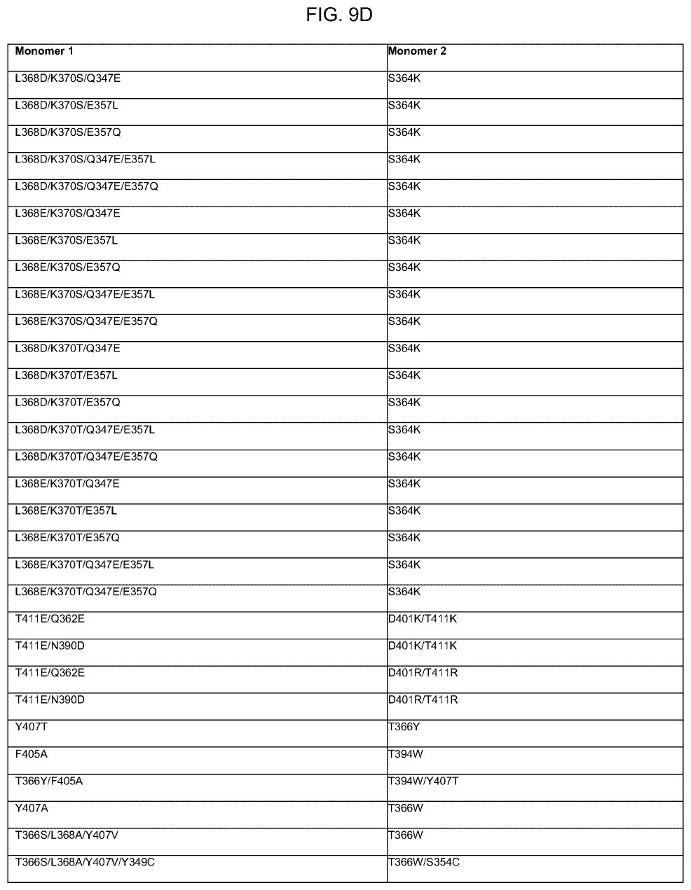

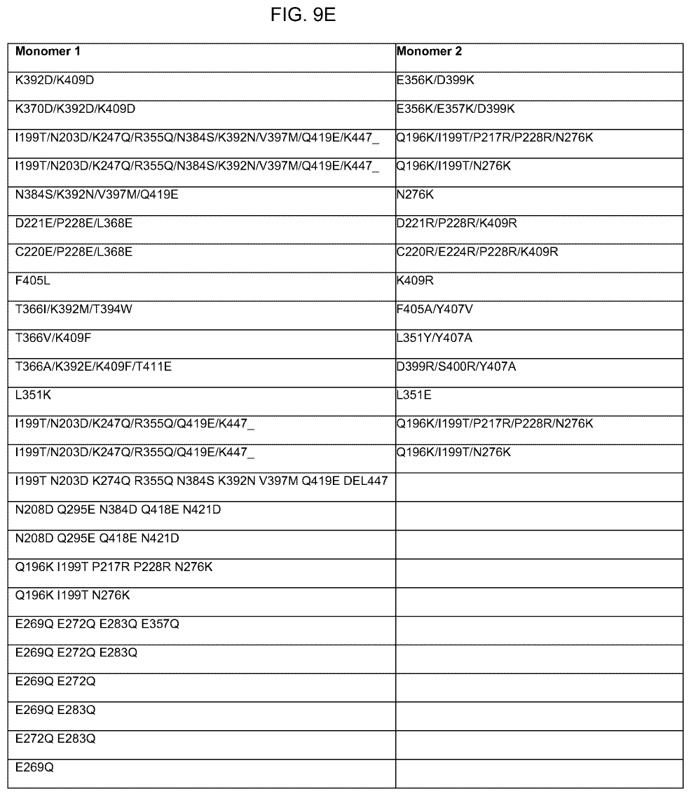

In some embodiments, the formation of heterodimers is facilitated by the addition of steric variants. That is, by changing amino acids in each heavy chain, different heavy chains are more likely to associate to form the heterodimeric structure than to form homodimers with the same Fc amino acid sequences. Suitable examples of steric variants are included in FIG. 9.

One mechanism is generally referred to in the art as “knobs and holes,” referring to amino acid engineering that creates steric influences to favor heterodimeric formation and disfavor homodimeric formation, can also optionally be used. This is further described in U.S. Patent Publication No. 20130205756, Ridgway et al., Protein Engineering 9 (7): 617 (1996); Atwell et al., J. Mol. Biol. 1997 270:26; U.S. Pat. No. 8,216,805, all of which are hereby incorporated by reference in their entirety. The Figures identify a number of “monomer A-monomer B” pairs that rely on “knobs and holes.” In addition, as described in Merchant et al., Nature Biotech. 16:677 (1998), these “knobs and hole” mutations can be combined with disulfide bonds to skew formation to heterodimerization.

An additional mechanism that finds use in the generation of heterodimers is sometimes referred to as “electrostatic steering” as described in Gunasekaran et al., J. Biol. Chem. 285 (25): 19637 (2010), hereby incorporated by reference in its entirety. This is sometimes referred to herein as “charge pairs.” In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “steric variants.” These include, but are not limited to, D221E/P228E/L368E paired with D221R/P228R/K409R (i.e., these are monomer corresponding sets) and C220E/P228E/368E paired with C220R/E224R/P228R/K409R.

Additional monomer A and monomer B variants that can be combined with other variants, optionally and independently in any amount, such as pI variants outlined herein or other steric variants that are shown in FIG. 37 of U.S. Patent Publication No. 2012/0149876, the figure and legend and SEQ ID NOs of which are incorporated expressly by reference herein.

In some embodiments, the steric variants outlined herein can be optionally and independently incorporated with any pI variant (or other variants such as Fc variants, FcRn variants, etc.) into one or both monomers, and can be independently and optionally included or excluded from the proteins of the invention.

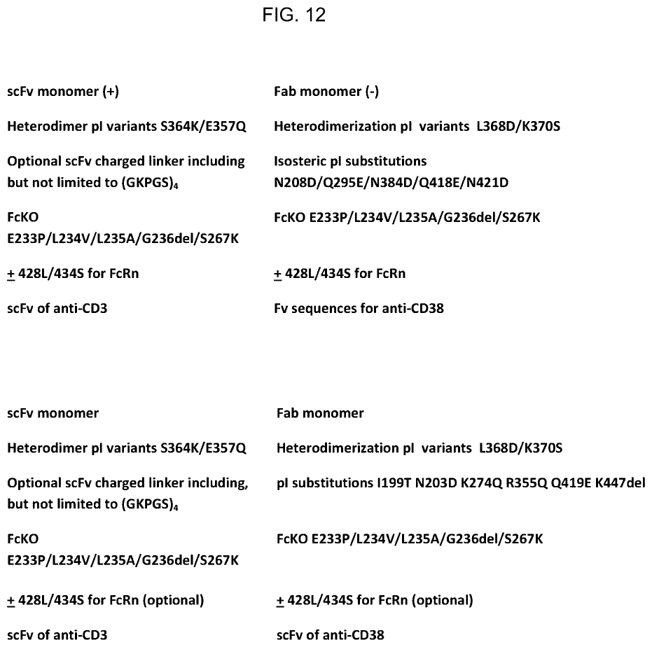

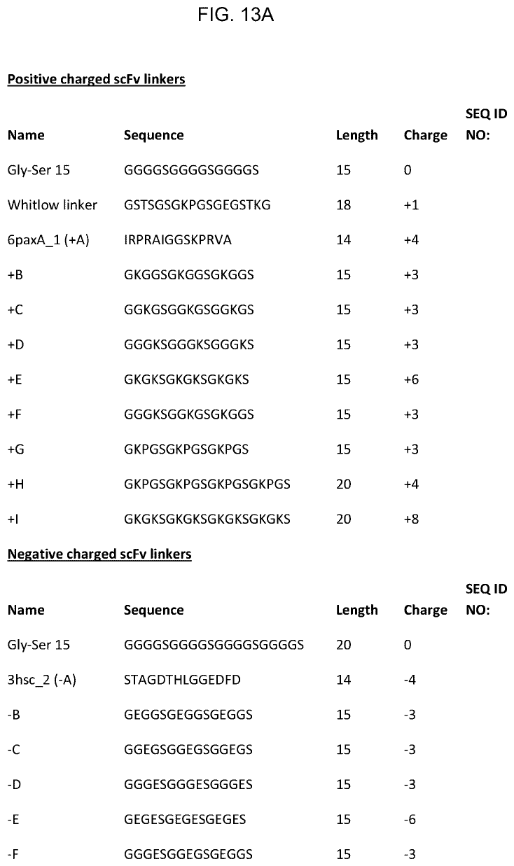

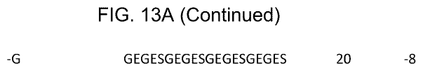

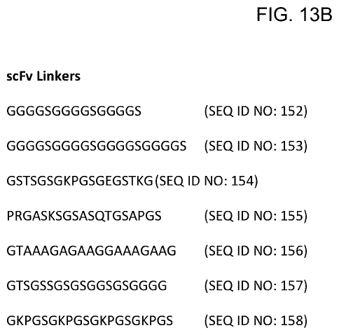

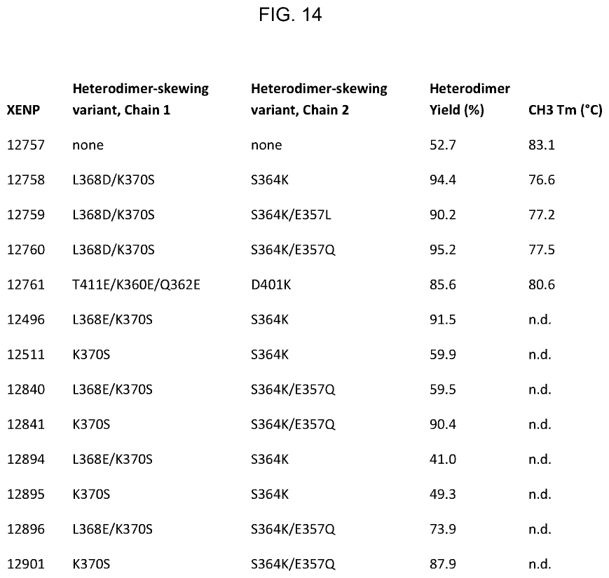

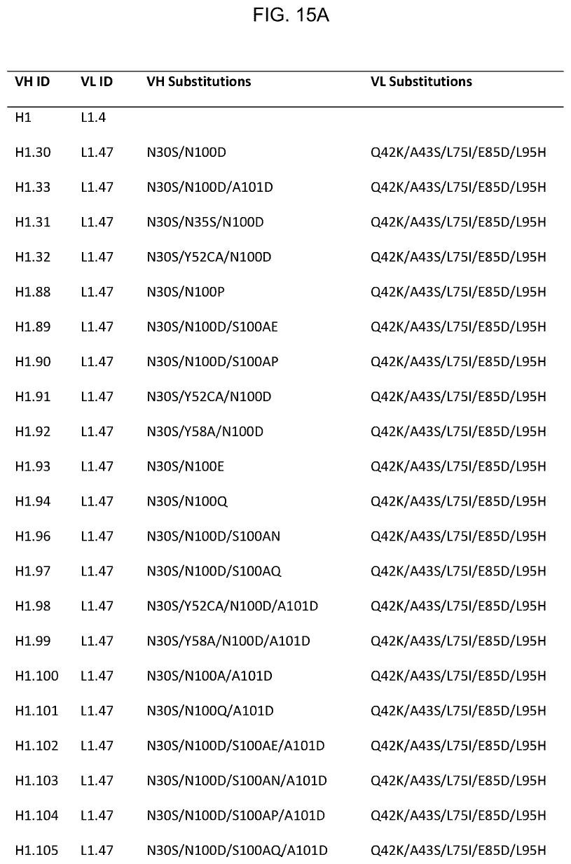

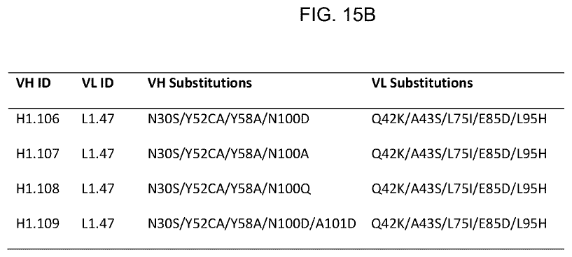

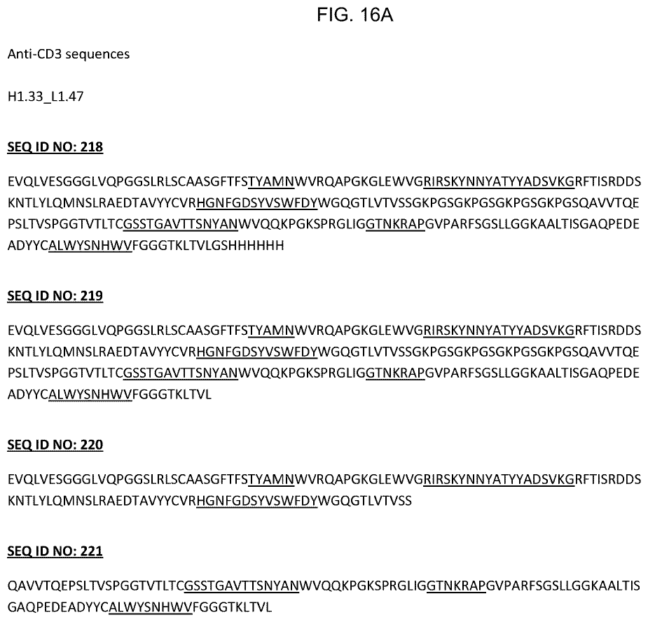

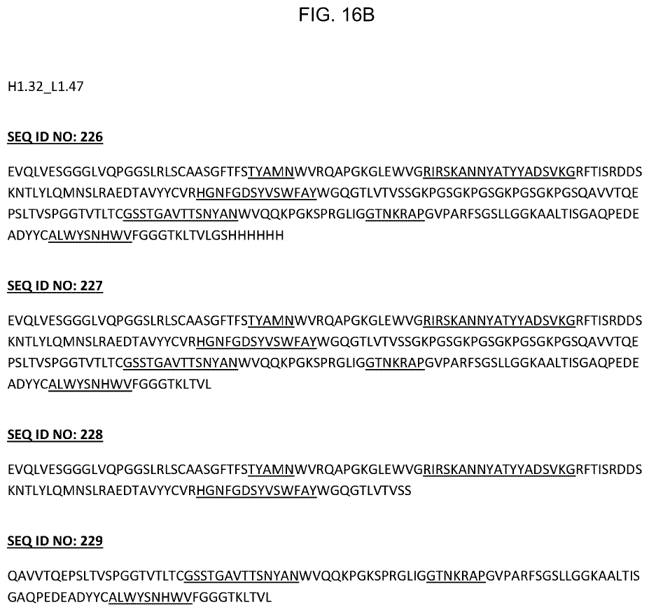

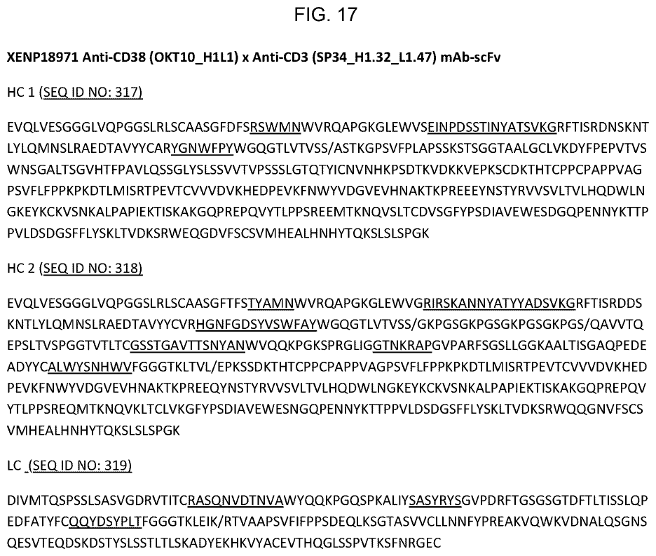

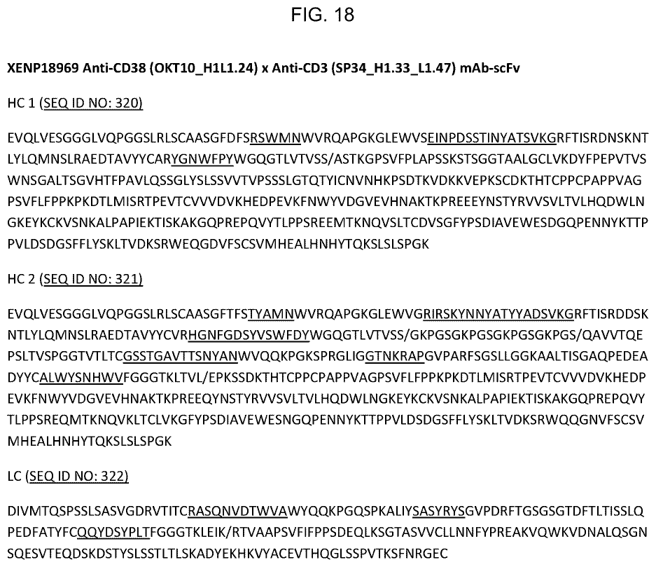

A list of suitable skew variants is found in FIG. 9 and FIG. 12. Of particular use in many embodiments are the pairs of sets including, but not limited to, S364K/E357Q: L368D/K370S; L368D/K370S: S364K; L368E/K370S: S364K; T411T/E360E/Q362E: D401K; L368D/K370S: S364K/E357L and K370S: S364K/E357Q. In terms of nomenclature, the pair “S364K/E357Q: L368D/K370S” means that one of the monomers has the double variant set S364K/E357Q and the other has the double variant set L368D/K370S.

### C. Additional Fc Variants for Adjusting Functionality

There are a number of useful Fc amino acid modification that can be made for a variety of reasons, including, but not limited to, altering binding to one or more FcγR receptors, altered binding to FcRn receptors, etc.

There are a number of useful Fc substitutions that can be made to alter binding to one or more of the FcγR receptors. Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the present invention include those listed in U.S. Patent Publication Nos. 2006/0024298 (particularly FIG. 41), 2006/0121032, 2006/0235208, 2007/0148170, all of which are expressly incorporated herein by reference in their entirety and specifically for the variants disclosed therein. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T.

In addition, there are additional Fc substitutions that find use in increased binding to the FcRn receptor and increased serum half life, as specifically disclosed in U.S. Patent Publication No. 2009/0163699, hereby incorporated by reference in its entirety, including, but not limited to, 434S, 434A, 428L, 308F, 259I, 428L/434S, 259I/308F, 436I/428L, 436I or V/434S, 436V/428L and 259I/308F/428L.

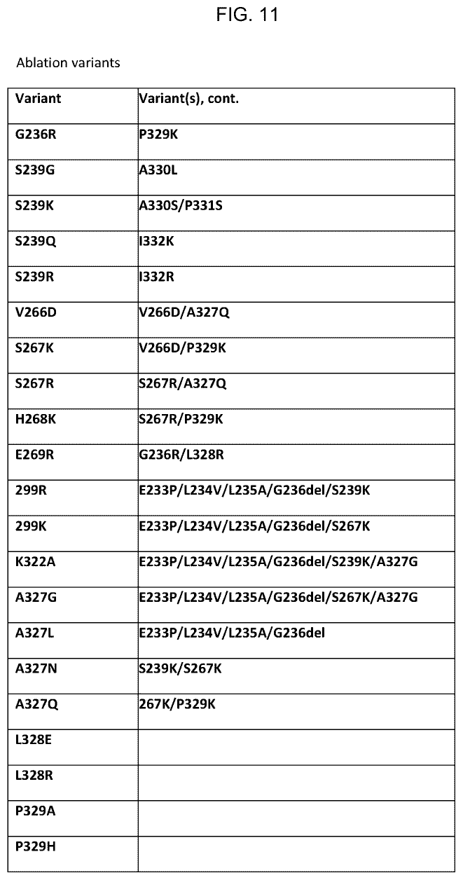

Another category of functional variants are “FcγR ablation variants” or “Fc knock out (FcKO or KO)” variants. For some therapeutic applications, it is desirable to reduce or remove the normal binding of the Fc domain to one or more or all of the Fcγ receptors (e.g., FcγR1, FcγRIIa, FcγRIIb, FcγRIIIa, etc.) to avoid additional mechanisms of action. That is, for example, particularly in the use of bispecific antibodies that bind CD3 monovalently, it may be desirable to ablate FcγRIIIa binding to eliminate or significantly reduce ADCC activity. Any level of reduction is contemplated (e.g., 50%, 60%, 70%, 80%, 90%, or 100% reduction in binding or activity). Examples of ablation variant modifications are depicted in FIG. 11, and each can be independently and optionally included or excluded, with preferred aspects utilizing ablation variants selected from the group consisting of G236R/L328R. E233P/L234V/L235A/G236del/S239K, E233P/L234V/L235A/G236del/S267K. E233P/L234V/L235A/G236del/S239K/A327G, E233P/L234V/L235A/G236del/S267K/A327G and E233P/L234V/L235A/G236del. It should be noted that the ablation variants referenced herein ablate FcγR binding but generally not FcRn binding.

### D. Additional Antibody Considerations

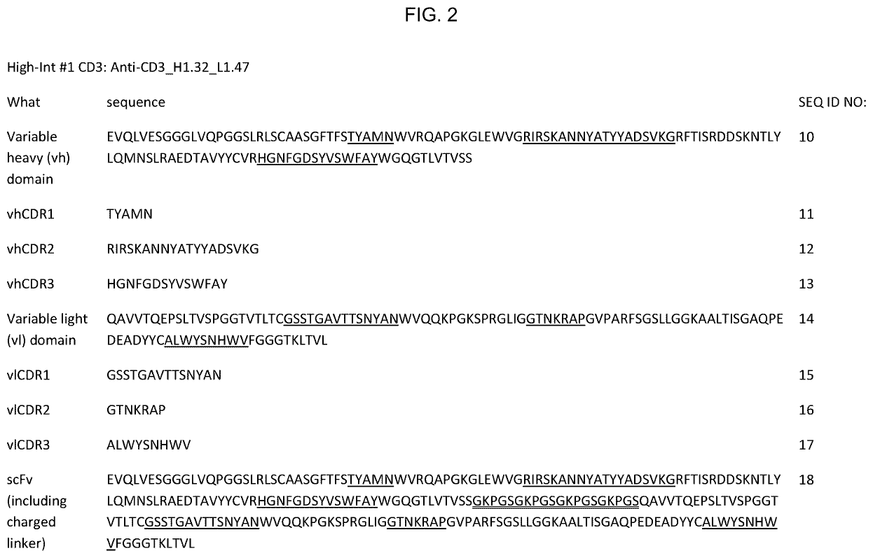

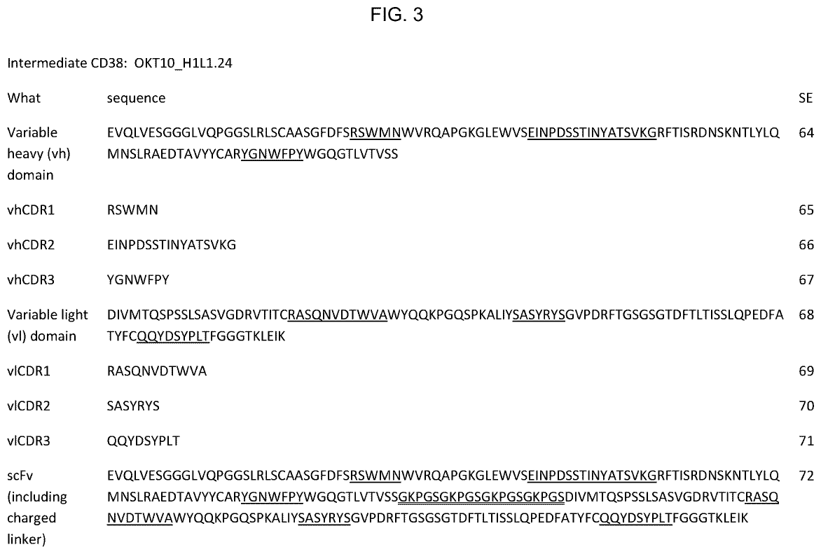

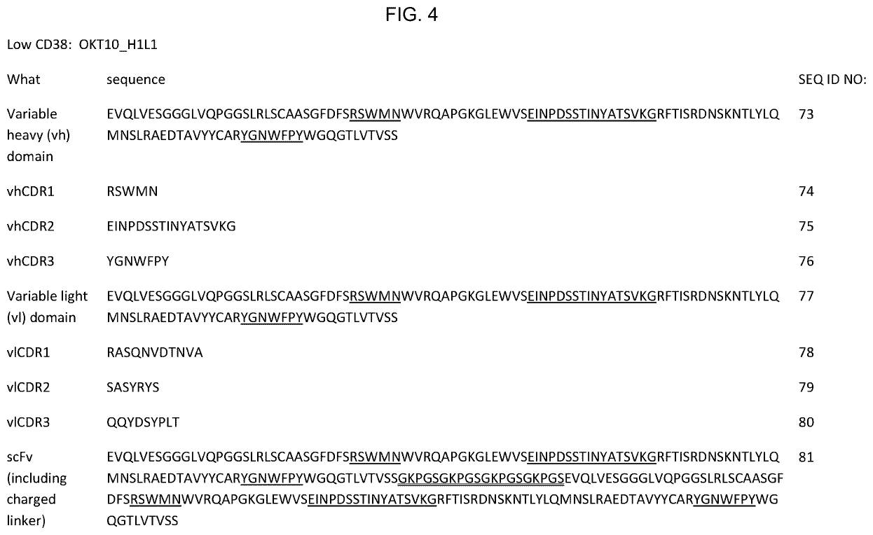

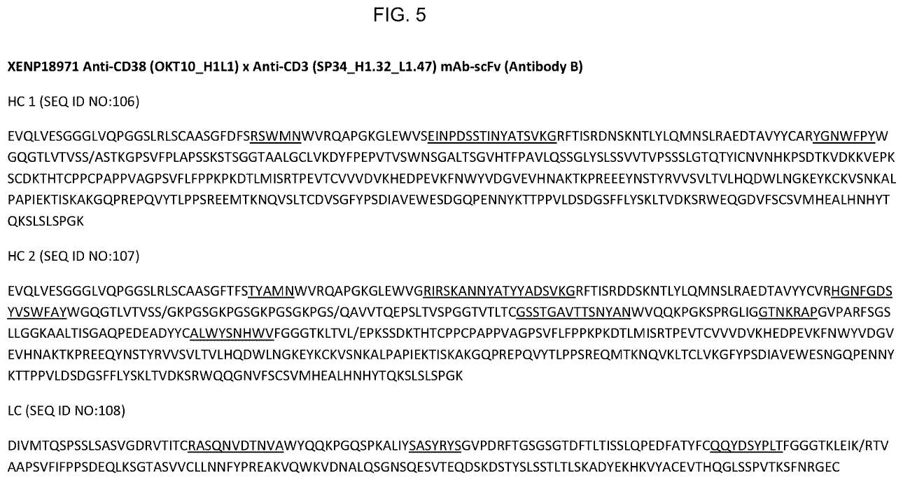

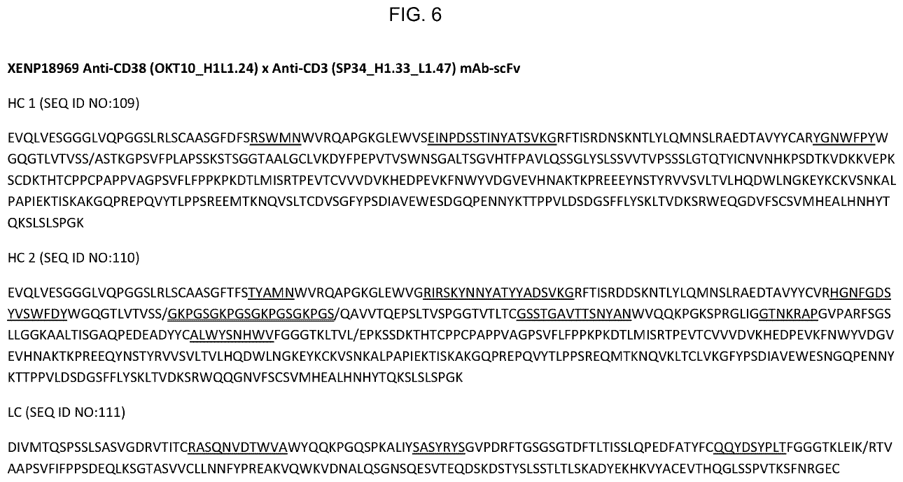

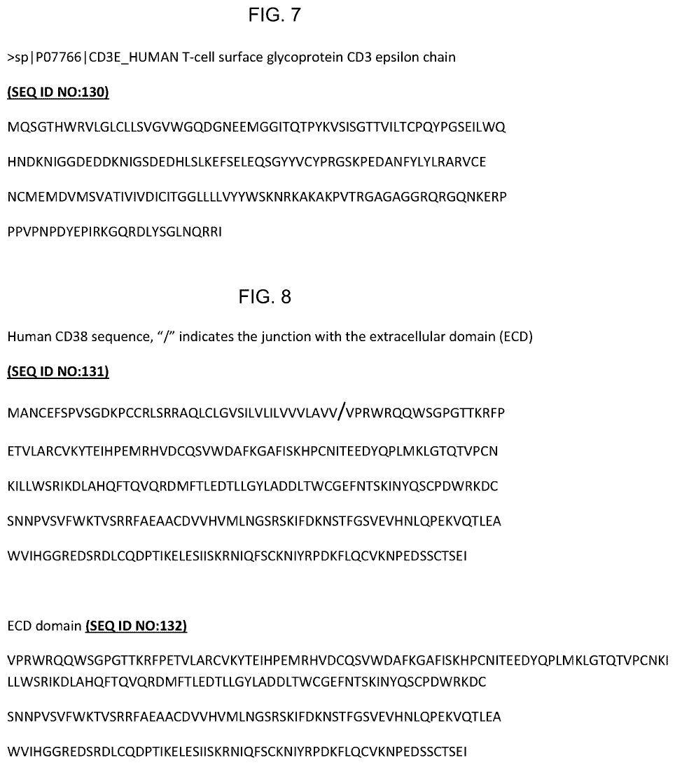

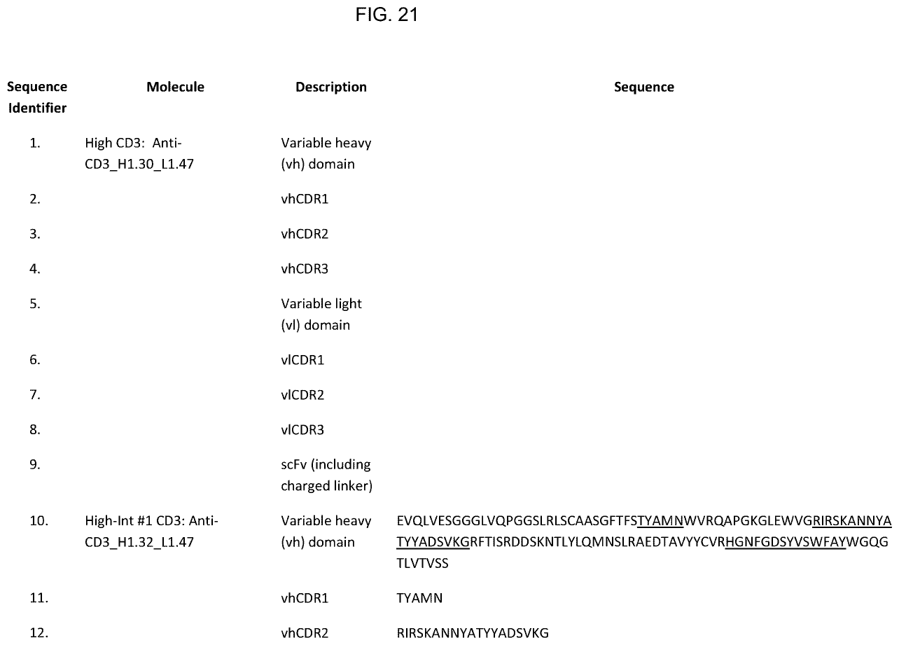

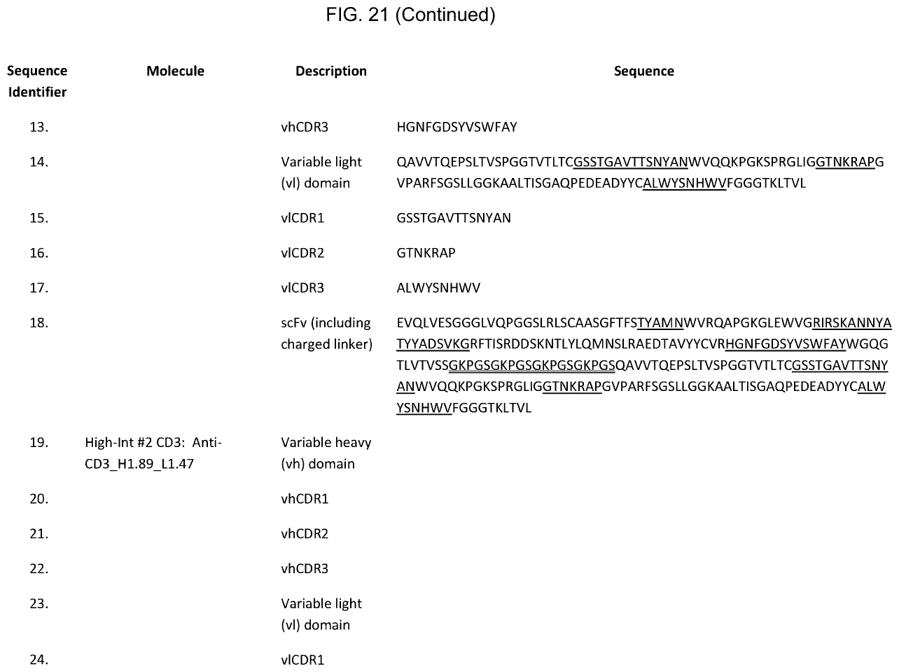

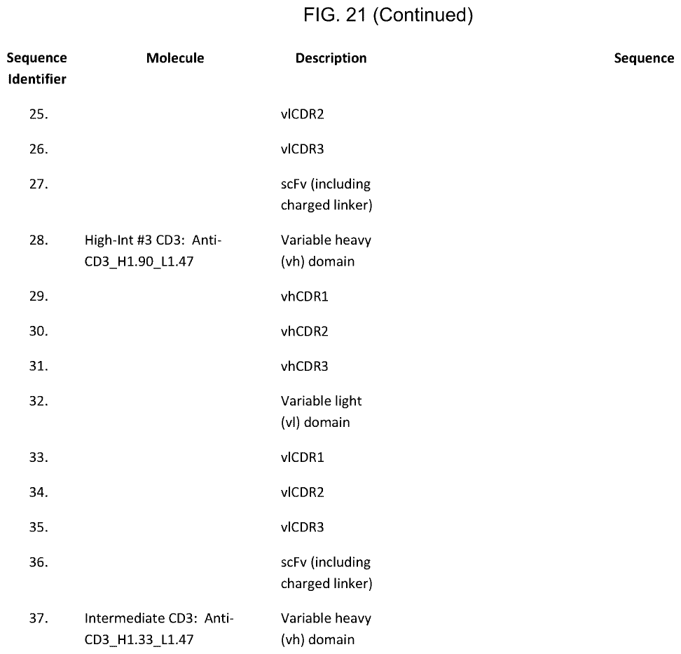

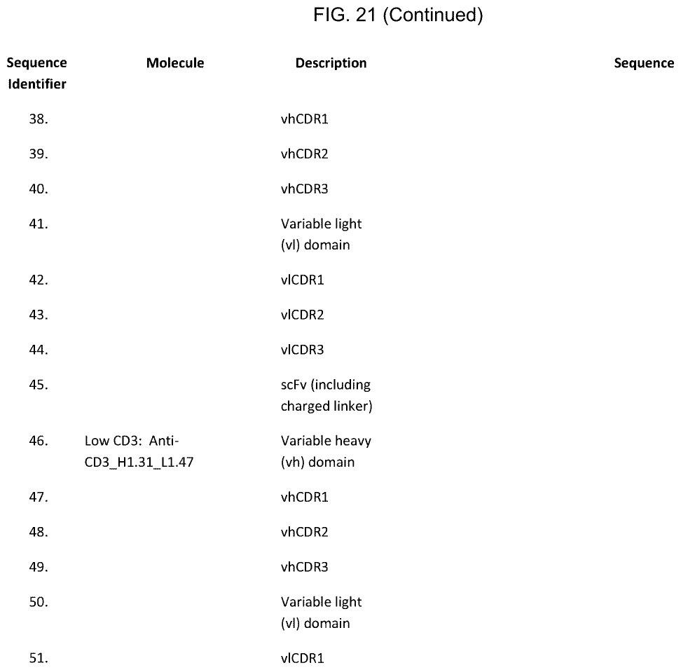

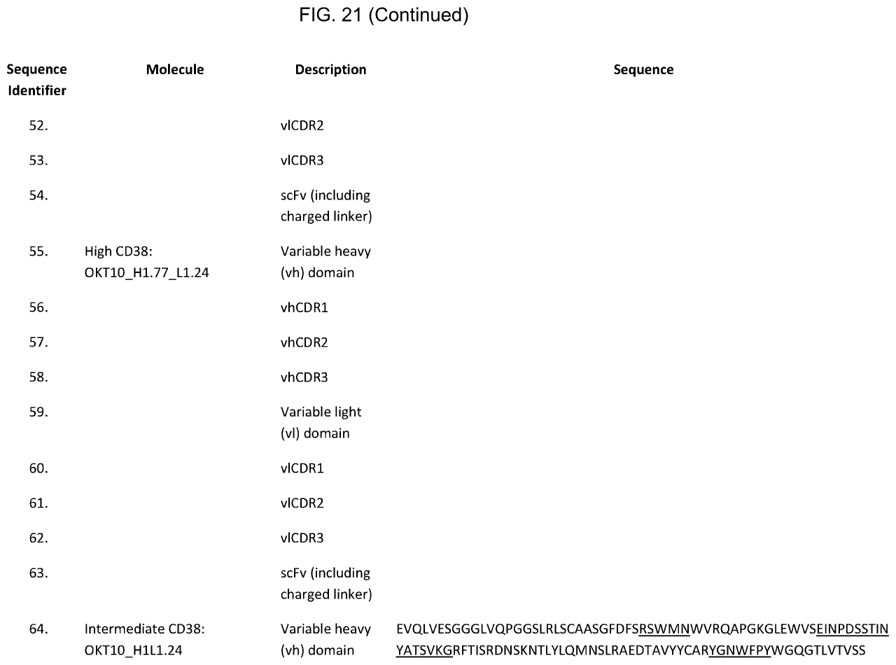

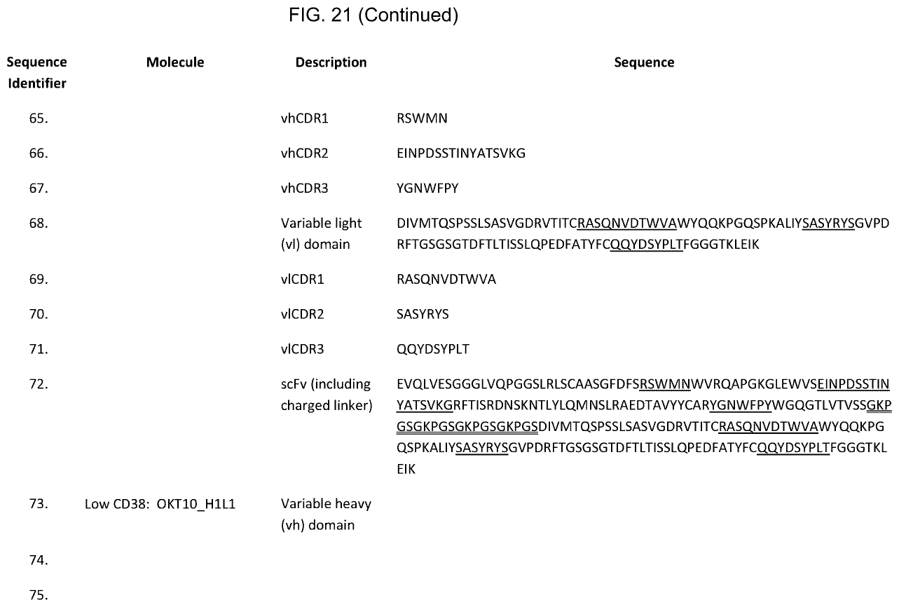

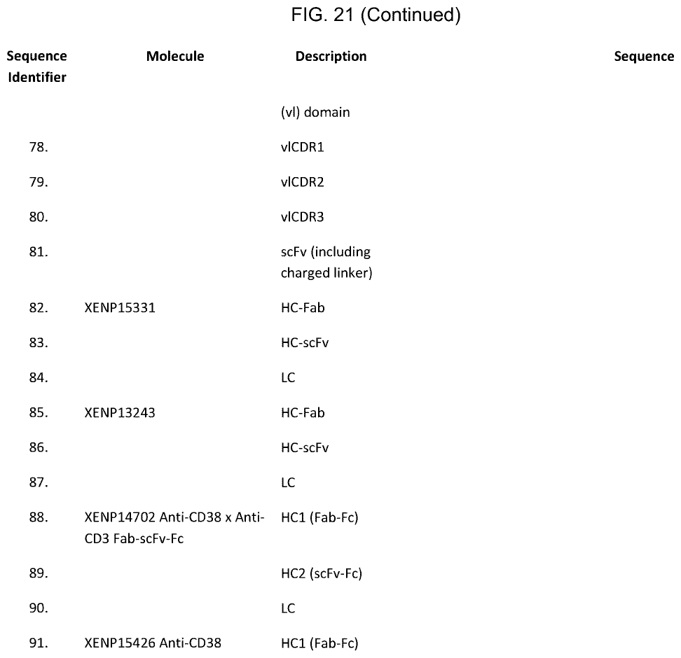

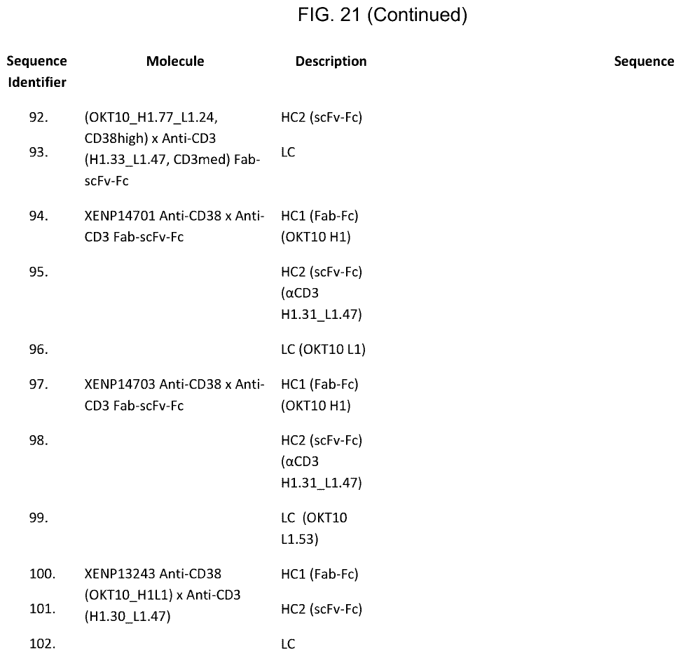

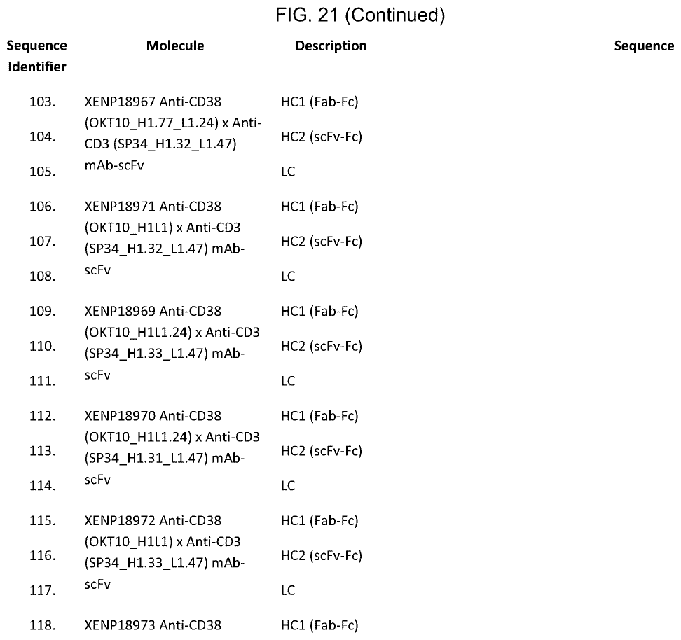

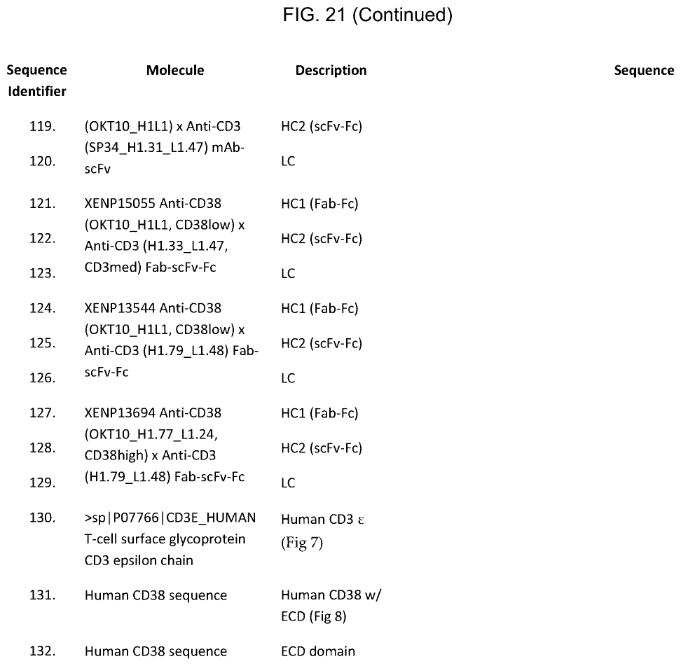

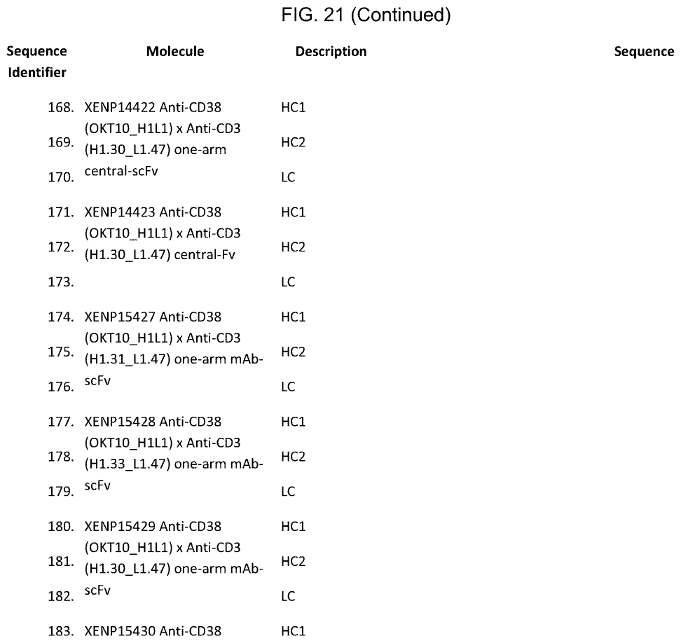

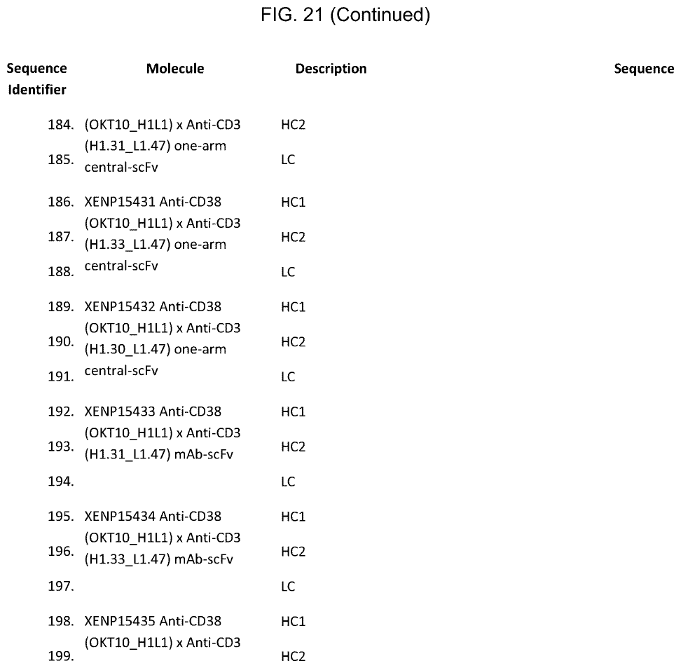

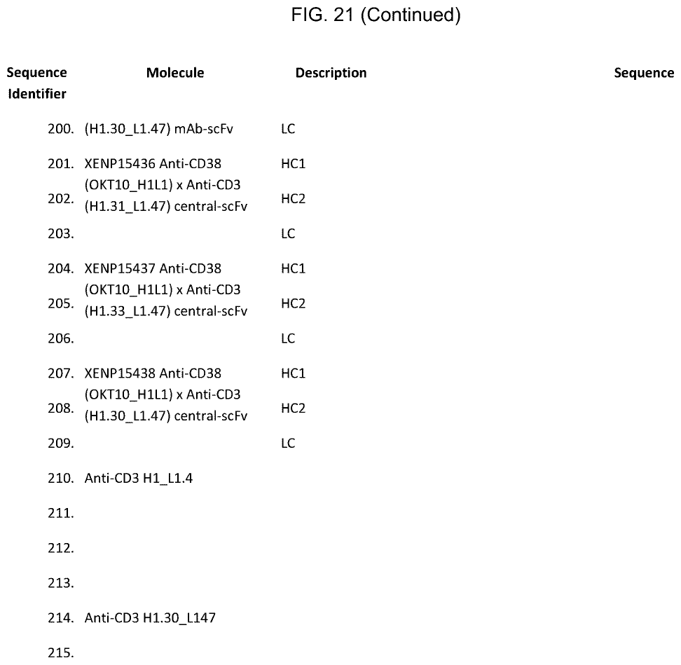

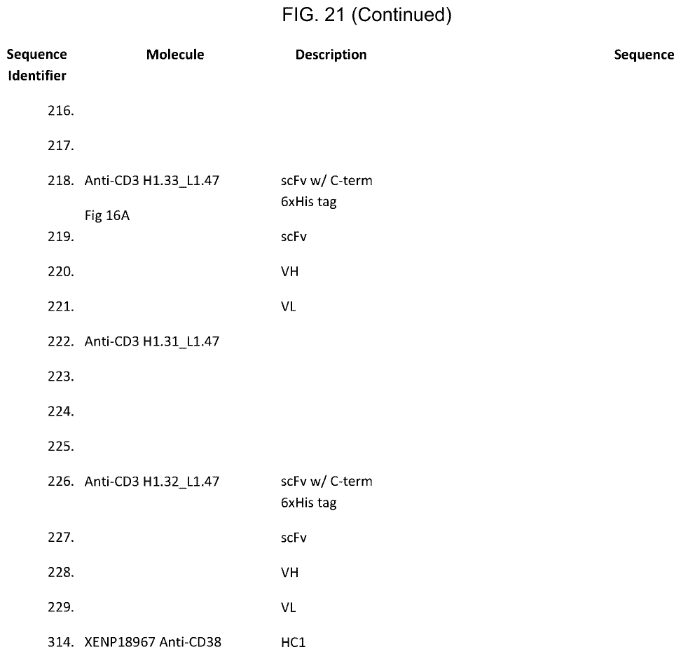

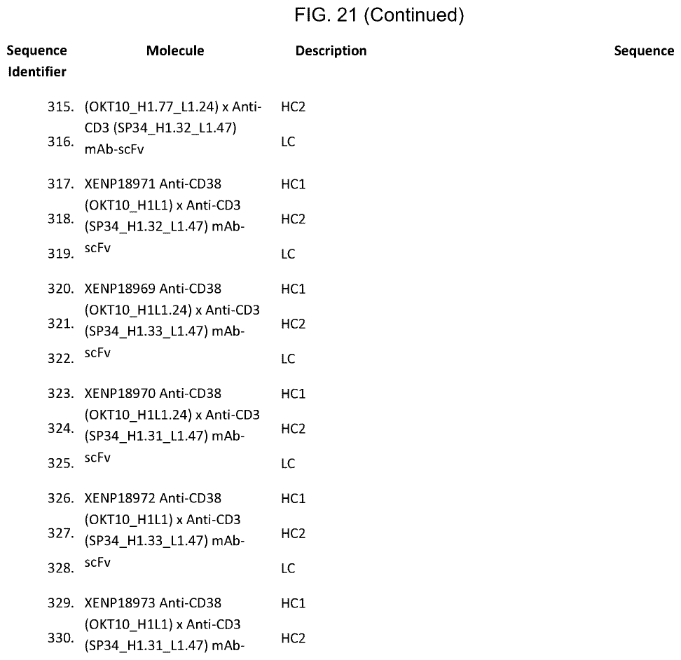

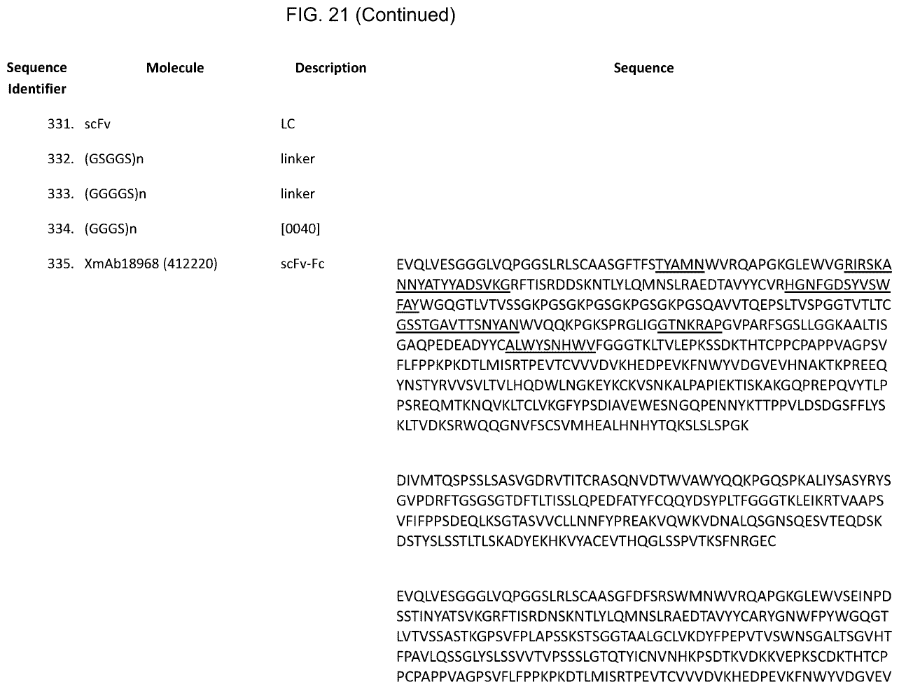

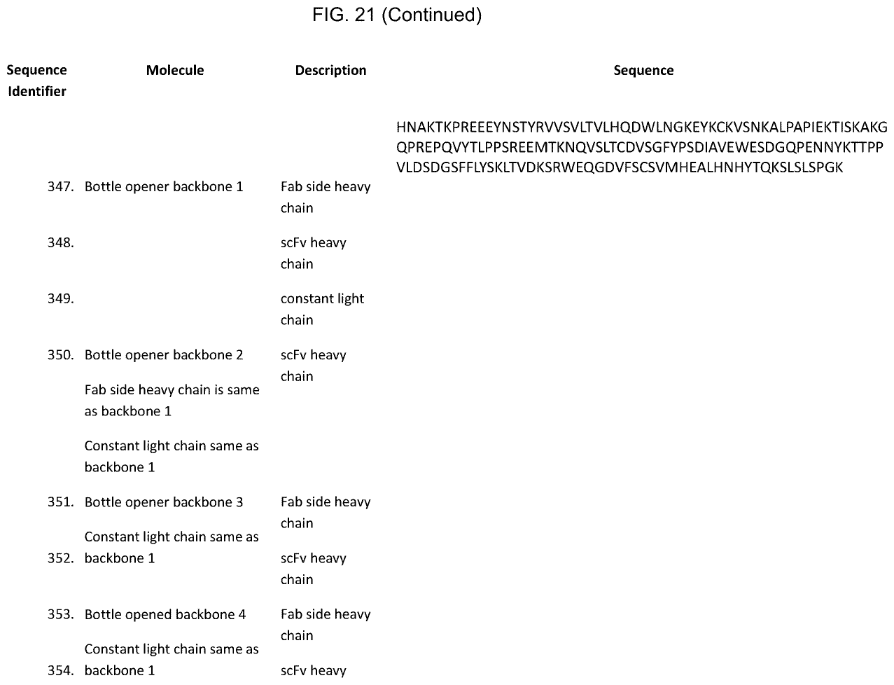

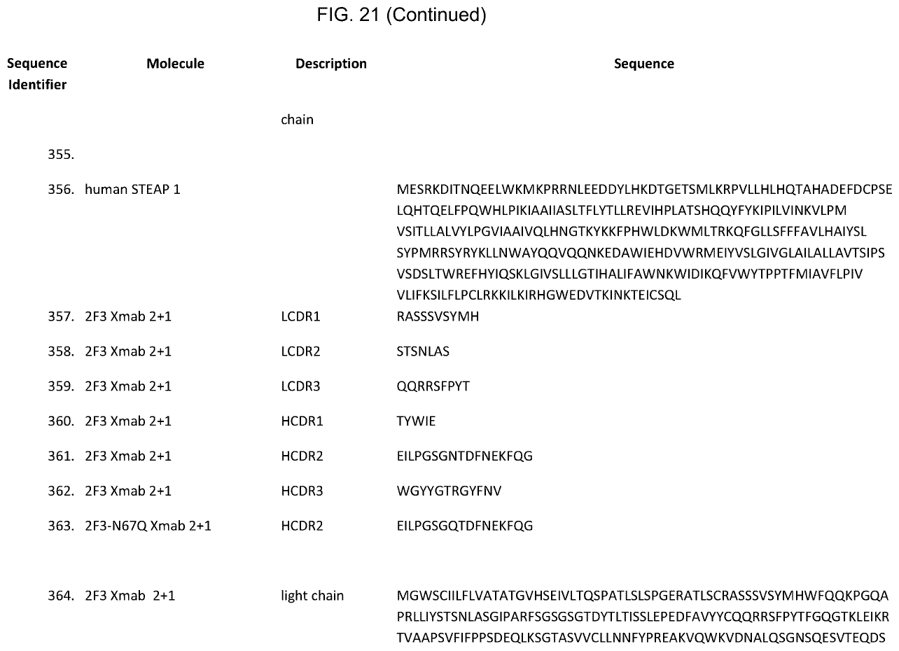

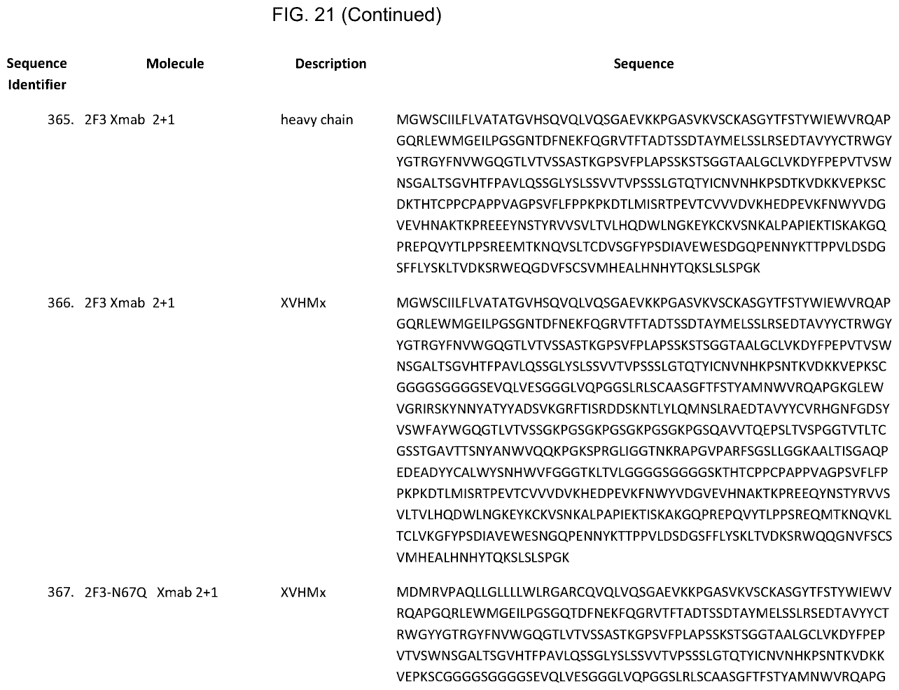

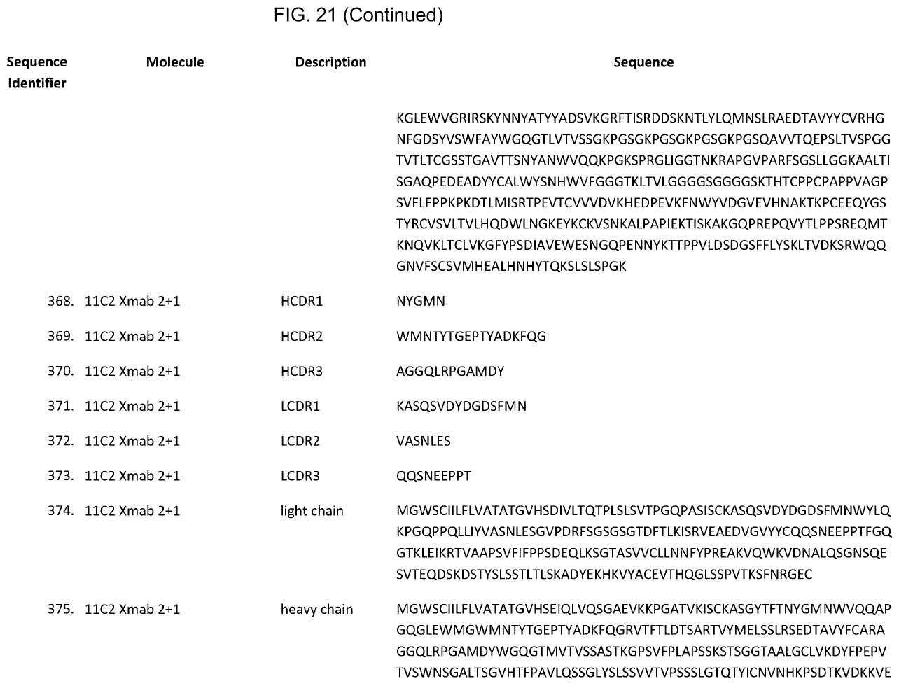

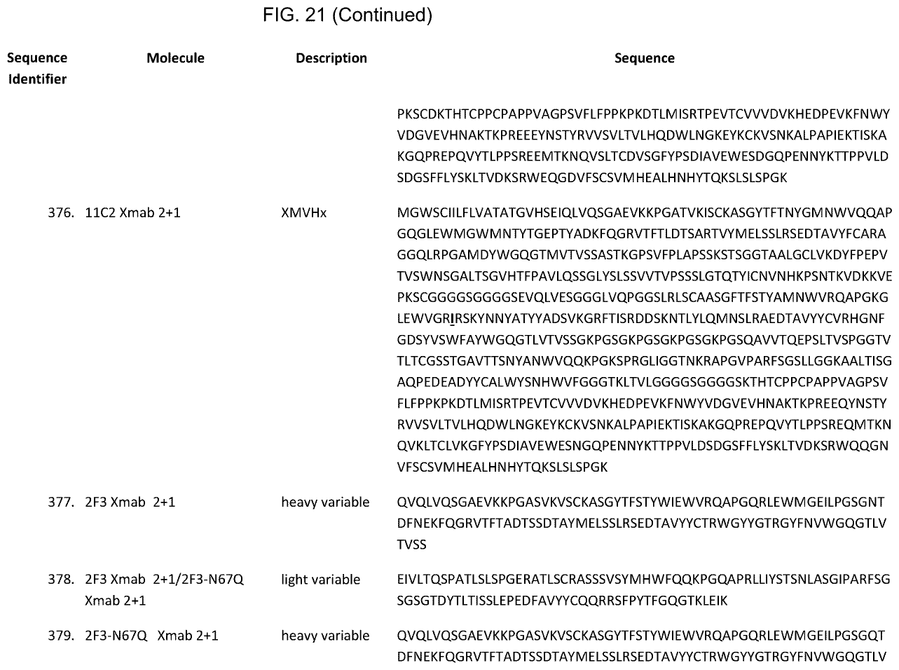

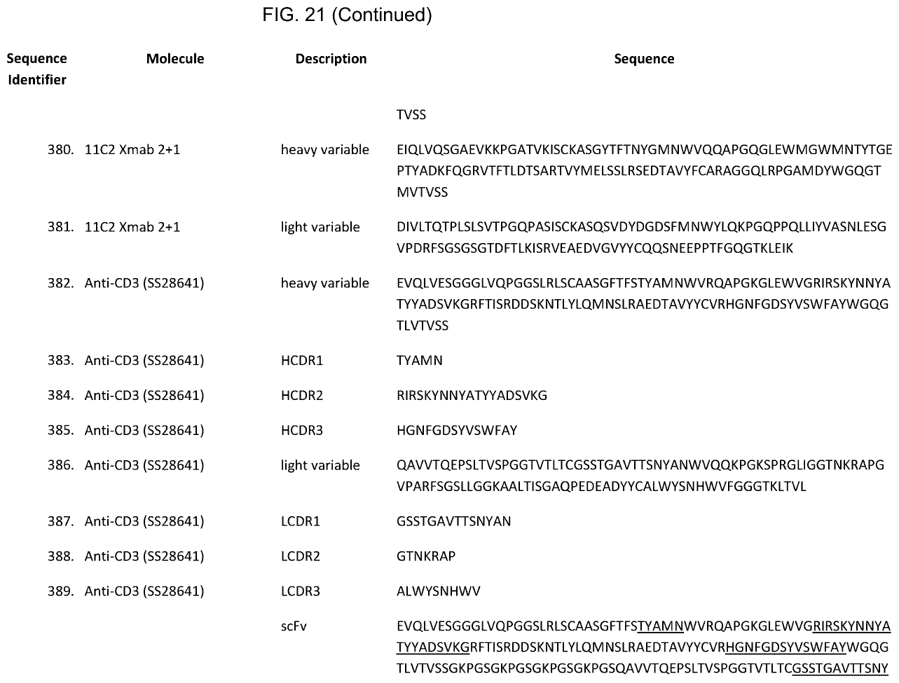

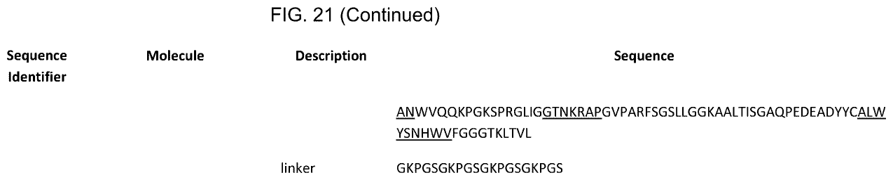

The disclosure contemplates the use of other heterodimeric antibodies in formulations described herein. For example, the variable heavy and light sequences, as well as the scFv sequences (and Fab sequences comprising these variable heavy and light sequences) described above can be used in other formats, such as those depicted in FIG. 2 of International Patent Publication No. 2014/145806 or FIG. 1 of International Patent Publication No. 2017/218707, the Figures, formats and legend of which is expressly incorporated herein by reference, as well as FIGS. 1A and 1B. Further, the amino acid sequences (e.g., CDR sequences, variable light and variable heavy chain sequences, and/or full length heavy and light chain sequences) of CD3-binding regions and CD38-binding regions are provided in the sequence listing provided herewith and summarized in FIG. 21. Any combination of the sequences referenced in FIG. 21 are contemplated herein so long as the resulting heterodimeric antibody engages both CD3 and CD38. Anti-CD3/anti-CD38 antibodies are further described in reference International Patent Publication No. WO 2016/086196; U.S. Patent Publication No. 20160215063; International Patent Publication No. WO 2017/091656; and U.S. Pat. No. 9,822,186, which are incorporated by reference herein in their entirety and particularly with respect to the description of anti-CD3/anti-CD38 antibodies and their amino acid and nucleic acid sequences, sequence listing, and Figures.

With respect to CD3 binding, the heterodimeric antibody may comprise an anti-CD3 antigen binding domain that has an intermediate or “medium” affinity to CD3. In this regard, the heterodimeric antibody binds to CD3 with an affinity (KD) of about 15-50 nM (e.g., about 16-50 nM, 15-45 nM, about 20-40 nM, about 25-40 nM, or about 30-40 nM), optionally measured using the assays described in U.S. Patent Publication No. 20160215063 and International Patent Publication No. WO 2017/091656, incorporated by reference herein.

In another aspect, the heterodimeric antibody of the method comprises an anti-CD3 antigen binding domain that is a “strong” or “high affinity” binder to CD3 (e.g., one example are heavy and light variable domains depicted as H1.30_L1.47 (optionally including a charged linker as appropriate)). In various embodiments, the antibody construct binds to CD3 with an affinity (KD) of about 3-15 nM (e.g., 3-10 nM or 4-7 nM), optionally measured using the assays described in U.S. Patent Publication No. 20160215063 and International Patent Publication No. WO 2017/091656, incorporated by reference herein. In other embodiments, the method employs a heterodimeric antibody comprising an anti-CD3 antigen binding domain that is a “lite” or “lower affinity” binder to CD3. In this regard, the heterodimeric antibody optionally binds to CD3 with an affinity (KD) of about 51 nM or more (e.g., 51-100 nM), optionally measured using the assays described in in U.S. Patent Publication No. 20160215063 and International Patent Publication No. WO 2017/091656, incorporated by reference herein. The heterodimeric antibody also binds, e.g., CD38 or STEAP1.

The affinity for CD38 of a bispecific antibody also has an effect on the efficacy of the antibody in targeting cells expressing CD38. Bispecific antibodies having “medium” or “low” affinity for CD38 are able to efficiently kill target cells in vitro and in vivo with reduced toxicity profiles. In various embodiments, bispecific antibodies demonstrating “high” affinity for CD38 bind to CD38 with an affinity (KD), e.g., below 1 nM; bispecific antibodies demonstrating “medium” or “intermediate” affinity for CD38 bind CD38 with an affinity (KD) of about, e.g., 1-10 nM (e.g., 2-8 nM or 3-7 nM); bispecific antibodies demonstrating “low” or “lite” affinity for CD38 bind CD38 with an affinity (KD) of about, e.g., 11 nM or more (such as 11-100 nM), all optionally measured using the methods set forth in U.S. Patent Publication No. 20160215063 and International Patent Publication No. WO 2017/091656, incorporated by reference herein.

Generally, specific binding can be exhibited, for example, by an antibody having a KD for an antigen of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen. Also, specific binding for a particular antigen can be exhibited, for example, by an antibody having a KA or Ka for an antigen or epitope of at least 20-, 50-, 100-, 500-, 1000-, 5,000-, 10,000- or more times greater for the antigen relative to a control, where KA or Ka refers to an association rate of a particular antibody-antigen interaction. It will be understand that disclosure relating to antibody also applies to antigen-binding protein.

Optionally, the heterodimeric antibody comprises a substitution of the cysteine at position 220 for serine; generally this is on the “scFv monomer” side of the heterodimeric antibody, although it can also be on the “Fab monomer” side, or both, to reduce disulfide formation. Specifically included within the sequences herein are one or both of these cysteines replaced (C220S).

### E. Fragments

The disclosure also contemplates the use of antibody fragments (distinguished from a full length antibody which constitutes the natural biological form of an antibody, including variable and constant regions, which generally include Fab and Fc domains alongside optional extra antigen binding domains such as scFvs). The antibody fragment contains at least one constant domain which can be engineered to produce heterodimers, such as pI engineering. Other antibody fragments that can be used include fragments that contain one or more of the CH1, CH2, CH3, hinge and CL domains of the invention that have been pI engineered.

### F. Chimeric/Humanized

The heterodimeric antibody can be a mixture from different species, e.g., a chimeric antibody and/or a humanized antibody. In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., International Patent Publication No. WO 92/11018, Jones, 1986, Nature 321:522-525, and Verhocyen et al., 1988, Science 239:1534-1536, all entirely incorporated by reference. “Backmutation” of selected acceptor framework residues to the corresponding donor residues is often required to regain affinity that is lost in the initial grafted construct (U.S. Pat. Nos. 5,530,101; 5,585,089; 5,693,761; 5,693,762; 6,180,370; 5,859,205; 5,821,337; 6,054,297; and 6,407,213, all entirely incorporated by reference). The humanized antibody also may comprise at least a portion of an immunoglobulin constant region, typically that of a human immunoglobulin, and thus will typically comprise a human Fc region. Humanized antibodies can also be generated using mice with a genetically engineered immune system. Roque et al., 2004, Biotechnol. Prog. 20:639-654, entirely incorporated by reference. A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein, all entirely incorporated by reference). Humanization methods include but are not limited to methods described in Jones et al., 1986, Nature 321:522-525; Riechmann et al., 1988; Nature 332:323-329; Verhoeyen et al., 1988, Science, 239:1534-1536; Queen et al., 1989, Proc Natl Acad Sci, USA 86:10029-33; He et al., 1998, J. Immunol. 160:1029-1035; Carter et al., 1992, Proc Natl Acad Sci USA 89:4285-9, Presta et al., 1997, Cancer Res. 57 (20): 4593-9; Gorman et al., 1991, Proc. Natl. Acad. Sci. USA 88:4181-4185; O'Connor et al., 1998, Protein Eng 11:321-8, all entirely incorporated by reference. Humanization or other methods of reducing the immunogenicity of nonhuman antibody variable regions may include resurfacing methods, as described for example in Roguska et al., 1994, Proc. Natl. Acad. Sci. USA 91:969-973, entirely incorporated by reference.

### Dosages

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts or doses effective for this use will depend on the condition to be treated (the indication), the delivered antibody construct, the therapeutic context and objectives, the severity of the disease, prior therapy, the patient's clinical history and response to the therapeutic agent, the route of administration, the size (body weight, body surface or organ size) and/or condition (the age and general health) of the patient, and the general state of the patient's own immune system. The proper dose can be adjusted according to the judgment of the attending physician such that it can be administered to the patient once or over a series of administrations, and in order to obtain the optimal therapeutic effect.

A therapeutic effective amount of an antigen-binding protein (e.g., antibody) preferably results in a decrease in severity of disease symptoms, an increase in frequency or duration of disease symptom-free periods or a prevention of impairment or disability due to the disease affliction. For treating target cell antigen-expressing tumors, a therapeutically effective amount of the antigen-binding protein (e.g., antibody), e.g., an anti-target cell antigen/anti-CD3 antibody construct, preferably inhibits cell growth or tumor growth by at least about 20%, at least about 40%, at least about 50%, at least about 60%, at least about 70%, at least about 80%, or at least about 90% relative to untreated patients. The ability of a molecule to inhibit tumor growth may be evaluated in an animal model predictive of efficacy.

The term “effective and non-toxic dose” refers to a tolerable dose of an antigen-binding protein (e.g., antibody) which is high enough to cause depletion of pathologic cells, tumor elimination, tumor shrinkage or stabilization of disease without or essentially without major toxic effects. Such effective and non-toxic doses may be determined, e.g., by dose escalation studies described in the art and should be below the dose inducing severe adverse side events (dose limiting toxicity, DLT).

The term “toxicity” as used herein refers to the toxic effects of a drug manifested in adverse events or severe adverse events. These side events might refer to a lack of tolerability of the drug in general and/or a lack of local tolerance after administration. Toxicity could also include teratogenic or carcinogenic effects caused by the drug.

The term “safety,” “in vivo safety” or “tolerability” defines the administration of a drug without inducing severe adverse events directly after administration (local tolerance) and during a longer period of application of the drug. “Safety.” “in vivo safety” or “tolerability” can be evaluated, e.g., at regular intervals during the treatment and follow-up period. Measurements include clinical evaluation, e.g., organ manifestations, and screening of laboratory abnormalities. Clinical evaluation may be carried out and deviations to normal findings recorded/coded according to NCI-CTC and/or MedDRA standards. Organ manifestations may include criteria such as allergy/immunology, blood/bone marrow, cardiac arrhythmia, coagulation and the like, as set forth, e.g., in the Common Terminology Criteria for adverse events v3.0 (CTCAE). Laboratory parameters which may be tested include for instance hematology, clinical chemistry, coagulation profile and urine analysis and examination of other body fluids such as serum, plasma, lymphoid or spinal fluid, liquor and the like. Safety can thus be assessed, e.g., by physical examination, imaging techniques (i.e., ultrasound, x-ray, CT scans, Magnetic Resonance Imaging (MRI)), other measures with technical devices (i.e. electrocardiogram), vital signs, by measuring laboratory parameters and recording adverse events. For example, adverse events in non-chimpanzee primates in the uses and methods according to the invention may be examined by histopathological and/or histochemical methods.

The above terms are also referred to e.g. in the Preclinical safety evaluation of biotechnology-derived pharmaceuticals S6; ICH Harmonised Tripartite Guideline; ICH Steering Committee meeting on Jul. 16, 1997.

A typical dosage may range from about 0.1 μg/kg to up to about 30 mg/kg or more, depending on the factors mentioned above. In specific embodiments, the dosage may range from 1.0 μg/kg up to about 20 mg/kg, optionally from 10 μg/kg up to about 10 mg/kg or from 100 μg/kg up to about 5 mg/kg. The formulation may be provided such that the heterodimeric antibody is provided in a unit dose, e.g., to achieve a dose in the range of 0.1-50 mg of antibody per kilogram of body weight (calculating the mass of the protein alone, without chemical modification).

### Therapeutic Use of the Formulation

The formulations described herein are useful as pharmaceutical compositions in the treatment, amelioration and/or prevention of the pathological medical condition as described herein in a patient in need thereof. The term “treatment” refers to both therapeutic treatment and prophylactic or preventative measures. Treatment includes the application or administration of the formulation to the body, an isolated tissue, or cell from a patient who has a disease/disorder, a symptom of a disease/disorder, or a predisposition toward a disease/disorder, with the purpose to cure, heal, alleviate, relieve, alter, remedy, ameliorate, improve, or affect the disease, the symptom of the disease, or the predisposition toward the disease.

The term “amelioration” as used herein refers to any improvement of the disease state of a patient having a tumor or cancer or a metastatic cancer as specified herein below, by the administration of composition comprising an antigen-binding protein described herein to a subject in need thereof. Such an improvement may also be seen as a slowing or stopping of the progression of the tumor or cancer or metastatic cancer of the patient. The term “prevention” as used herein means the avoidance of the occurrence or re-occurrence of a patient having a tumor or cancer or a metastatic cancer as specified herein below, by the administration of the composition comprising an antigen-binding protein (i.e., antibody construct) described herein to a subject in need thereof.

In one embodiment the invention provides a method for the treatment or amelioration of a proliferative disease, a tumorous disease, a viral disease or an immunological disorder, comprising the step of administering to a subject in need thereof the formulation described herein. The term “disease” refers to any condition that would benefit from treatment with the pharmaceutical composition described herein. This includes chronic and acute disorders or diseases including those pathological conditions that predispose the mammal to the disease in question. The term “viral disease” describes diseases, which are the result of a viral infection of a subject. The term “immunological disorder” is used herein in line with the common definition of this term, which includes immunological disorders such as autoimmune diseases, hypersensitivities, immune deficiencies.

“Neoplasm” is an abnormal growth of tissue, usually but not always forming a mass. When also forming a mass, it is commonly referred to as a “tumor.” Neoplasms or tumors or can be benign, potentially malignant (pre-cancerous), or malignant. Malignant neoplasms are commonly called cancer. They usually invade and destroy the surrounding tissue and may form metastases, i.e., they spread to other parts, tissues or organs of the body. Hence, the term “metastatic cancer” encompasses metastases to other tissues or organs than the one of the original tumor. Lymphomas and leukemias are lymphoid neoplasms. For the purposes of the present invention, they are also encompassed by the terms “tumor” or “cancer.” The terms “subject in need” or those “in need of treatment” includes those already with the disorder, as well as those in which the disorder is to be prevented. The subject in need or “patient” includes human and other mammalian subjects that receive either prophylactic or therapeutic treatment.

### Routes of Administration

Exemplary routes of administration include, but are not limited to topical routes (such as epicutaneous, inhalational, nasal, ophthalmic, auricular/aural, vaginal, mucosal); enteral routes (such as oral, gastrointestinal, sublingual, sublabial, buccal, rectal); and parenteral routes (such as intravenous, intraarterial, intraosseous, intramuscular, intracerebral, intracerebroventricular, epidural, intrathecal, subcutaneous, intraperitoneal, extra-amniotic, intraarticular, intracardiac, intradermal, intralesional, intrauterine, intravesical, intravitreal, transdermal, intranasal, transmucosal, intrasynovial, intraluminal). Preferably, the pharmaceutical formulation is administered parenterally, e.g., intravenously, subcutaneously, or intramuscularly. Parenteral administration may be achieved by injection, such as bolus injection, or by infusion, such as continuous infusion. Administration may be achieved via depot for long-term release. In some embodiments, the formulation is administered intravenously by an initial bolus followed by a continuous infusion to maintain therapeutic circulating levels of drug product. In some embodiments, the formulation is administered as a one-time dose. Pharmaceutical compositions may be administered using a medical device. Examples of medical devices for administering pharmaceutical compositions are described in U.S. Pat. Nos. 4,475,196; 4,439,196; 4,447,224; 4,447,233; 4,486,194; 4,487,603; 4,596,556; 4,790,824; 4,941,880; 5,064,413; 5,312,335; 5,312,335; 5,383,851; and 5,399,163.

In particular, the present invention provides for an uninterrupted administration of the suitable composition. As a non-limiting example, uninterrupted or substantially uninterrupted, i.e., continuous administration may be realized by a small pump system worn by the patient for metering the influx of therapeutic agent into the body of the patient. The pharmaceutical composition can be administered by using said pump systems. Such pump systems are generally known in the art, and commonly rely on periodic exchange of cartridges containing the therapeutic agent to be infused. When exchanging the cartridge in such a pump system, a temporary interruption of the otherwise uninterrupted flow of therapeutic agent into the body of the patient may ensue. In such a case, the phase of administration prior to cartridge replacement and the phase of administration following cartridge replacement would still be considered within the meaning of the pharmaceutical means and methods of the invention together make up one “uninterrupted administration” of such therapeutic agent.

The continuous or uninterrupted administration of the formulation may be intravenous or subcutaneous by way of a fluid delivery device or small pump system including a fluid driving mechanism for driving fluid out of a reservoir and an actuating mechanism for actuating the driving mechanism. Pump systems for subcutaneous administration may include a needle or a cannula for penetrating the skin of a patient and delivering the suitable composition into the patient's body. Said pump systems may be directly fixed or attached to the skin of the patient independently of a vein, artery or blood vessel, thereby allowing a direct contact between the pump system and the skin of the patient. The pump system can be attached to the skin of the patient for 24 hours up to several days. The pump system may be of small size with a reservoir for small volumes. As a non-limiting example, the volume of the reservoir for the suitable pharmaceutical composition to be administered can be between 0.1 and 50 ml.

The continuous administration may also be transdermal by way of a patch worn on the skin and replaced at intervals. One of skill in the art is aware of patch systems for drug delivery suitable for this purpose. It is of note that transdermal administration is especially amenable to uninterrupted administration, as exchange of a first exhausted patch can advantageously be accomplished simultaneously with the placement of a new, second patch, for example on the surface of the skin immediately adjacent to the first exhausted patch and immediately prior to removal of the first exhausted patch. Issues of flow interruption or power cell failure do not arise.

If the pharmaceutical composition has been lyophilized, the lyophilized material is first reconstituted in an appropriate liquid prior to administration. The lyophilized material may be reconstituted in, e.g., bacteriostatic water for injection (BWFI), physiological saline, phosphate buffered saline (PBS), or the same formulation the protein had been in prior to lyophilization. The pharmaceutical composition can be administered as a sole therapeutic or in combination with additional therapies such as anti-cancer therapies as needed, e.g. other proteinaceous and non-proteinaceous drugs. These drugs may be administered simultaneously with the composition of the invention as defined herein or separately before or after administration of said formulation in timely defined intervals and doses.

### Kits

As an additional aspect, the described herein are kits which comprise one or more pharmaceutical compositions described herein packaged in a manner which facilitates their use for administration to subjects. In one embodiment, such a kit includes a formulation described herein (e.g., a composition comprising an antibody described therein), packaged in a container such as a sealed bottle, vessel, single-use or multi-use vial, prefilled syringe, or prefilled injection device, optionally with a label affixed to the container or included in the package that describes use of the compound or composition in practicing the method. In one aspect, the composition is packaged in a unit dosage form. The kit may further include a device suitable for administering the composition according to a specific route of administration. Preferably, the kit contains a label that describes use of an antibody described herein or formulation described herein.

The pharmaceutical compositions described herein can be formulated in various forms, e.g., in solid, liquid, frozen, gaseous or lyophilized form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts.

Generally, various storage and/or dosage forms are conceivable for the pharmaceutical composition of the invention, depending, i.e., on the intended route of administration, delivery format and desired dosage (see, for example, Remington's Pharmaceutical Sciences, 22nd edition, Oslo, A., Ed., (2012)). The skilled person will be aware that such choice of a particular dosage form may for example influence the physical state, stability, rate of in vivo release and rate of in vivo clearance of an antibody.

For instance, the primary vehicle or carrier in a pharmaceutical composition may be either aqueous or non-aqueous in nature. A suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles.

### EXAMPLES

### Materials and Methods

SE-UHPLC: Size Exclusion Ultra High Performance Liquid Chromatography is a method for quantitative analysis of recombinant monoclonal antibody (mAb) or X-mAb. SE-UHPLC separates proteins based on differences in their hydrodynamic volumes. Molecules with higher hydrodynamic volumes elute earlier than molecules with smaller volumes. The samples are loaded onto an SE-UHPLC column (BEH200, 4.6×300 mm, (Waters Corporation, 186005226)), separated isocratic ally and the eluent is monitored by UV absorbance. Purity is determined by calculating the percentage of each separated component as compared to the total integrated area. SE-UHPLC settings are as follows: Flow rate: 0.4 mL/min, Run time: 12 min, UV detection: 280 nm, Column temperature: Ambient, Target protein load: 6 μg, Protein compatible flow cell: 5 mm.

Cation Exchange High Performance Liquid Chromatography (CEX) is a method for quantitative purity analysis on the charged variant distribution. Mobile Phase A: 1×CX-1 pH gradient buffer A, pH 5.6 (10×CX-1 pH gradient buffer A, pH 5.6, 250 mL and Mobile Phase B: 1×CX-1 pH gradient buffer, pH 10.2.

CE-HPLC: Cation Exchange High Performance Liquid Chromatography is a method for quantitative purity analysis on the charged variant distribution. Mobile Phase A: 25 mM Sodium phosphate, 10% Acetonitrile, pH 6.7 and Mobile Phase B: 25 mM Sodium phosphate, 500 mM Sodium chloride, 10% Acetonitrile, pH 6.7. Column used for Antibody A CE-HPLC is a Bio Mab NP-5, 4.6×250 mm, 5 μm (Agilent Technology, 5190-2407). CE-HPLC method settings are as follows: Flow rate: 0.75 mL/min, Run time: 60 min, Column temperature set point: 30° C.±5° C., Detector wavelength: 280 nm, Target protein load: 20 μg. Column used for Antibody B CE-HPLC method is a YMC BioPro SP-F, 4.6×100 mm, 5 μm (YMC Co., Ltd., SF00S05-1046WP). CE-HPLC method settings are as follows: Flow rate: 1.0 mL/min, Run Time: 45 minutes, Autosampler temperature set point: 5±3° C., Column temperature set point: 30±2° C., Detector wavelength: 280 nm, Target sample protein load: 70±10 μg.

rCE-SDS: Reduced Capillary Electrophoresis-Sodium Dodecyl Sulfate is a method for quantitative purity analysis under denaturing and reducing conditions.

MAM: Multi Attribute Method is a method for multiple product quality attributes (PQA) (oxidation, isomerization, deamidation and glycation) using the Thermo Scientific Orbitrap type Mass Spectrometer and Chromeleaon Software. Chemical modification upon heat stress (incubation at 40° C.) was measured using peptide mapping. Proteins were enzymatically digested and the resulting peptides were separated using reversed phased chromatography. Proteins are denatured with guanidine HCl and then reduced with dithiotreitol (DTT). After incubation in DTT, free cysteine residues were alkylated by the addition of iodoacetic acid. Samples were then buffer exchanged into 50 mM Tris-HCl, 20 mM Methionine, pH7.8 for digestion. Trypsin and Elastase was added to separate reaction tubes (enzyme to protein ratio 1:20). Samples were digested for 60 min at 37° C. and 30 min at 37° C. respectively. Digestion was quenched by adding Guanidine HCl, 250 mM Acetate, pH4.7.

Moisture content of lyophilized drug product is determined by a calorimetric titration with an oven. Moisture limit for lyophilized drug product is 2%. The Karl Fischer method's principle is based on the water content in the sample determined by means of calorimetric titration. Water is released by heating the sample in an oven. Dry air or inert gas such as nitrogen carried the evaporated moisture to the titrator. The amount of water present is determined by measuring the amount of coulombs (current/time) generated during the titration. When all the water has been consumed by titration, an excess of iodine occurs. The end point is indicated volumetrically by applying an alternating current of constant strength to a double Pt electrode. This results in a voltage difference between Pt wired of the indicator electrode, which is drastically lowered in the presence of minimal quantities of free iodine. This voltage difference is used to determine the end point of the titration.

### Example 1—Antibody Stability in Low pH Formulations

The following Example describes assays to verify the stability of a heterodimeric antibody described herein for up to 3 years at various different temperatures (4° C., 25° C., 40° C., −30° C. &−40° C.) in either a liquid formulation or a lyophilized formulation at different protein concentrations (i.e., 1 mg/mL and 5 mg/mL). The stability of the heterodimeric antibody was analyzed using the following assays: Appearance (via visual inspection of 20 vials at each time point), pH, Osmolality, CE-HPLC, rCE, MAM (Multi Attribute Method) and Karl Fischer (moisture content). The verification study samples were a 1.3 mL fill in 5cc vials. Heterodimeric antibody DS (material without polysorbate 80) was at 10.6 mg/mL and was diluted with buffer (G42Su) to reach 5 mg/mL and 1 mg/mL. The formulations were isotonic in the G42SuT formulation with an osmolality value of 326 mOsm/kg.

Stability of the heterodimer antibody was assessed in the following formulations:
Formulation A: 1 mg/mL heterodimeric antibody lyophilized formulation in 10 mM L-Glutamic acid, 9% (w/v) Sucrose, 0.01% (w/v) Polysorbate 80, pH4.2;
Formulation B: 5 mg/mL heterodimeric antibody lyophilized formulation in 10 mM L-Glutamic acid, 9% (w/v) Sucrose, 0.01% (w/v) Polysorbate 80, pH4.2;
Formulation C: 5 mg/mL heterodimeric antibody liquid formulation in 10 mM L-Glutamic acid, 9% (w/v) Sucrose, 0.01% (w/v) Polysorbate 80, pH4.2; and
Formulation D: 1 mg/mL heterodimeric antibody liquid formulation in 10 mM Acetate, 9% (w/v) Sucrose, 0.01% Polysorbate 80, pH5.2).

### Results:

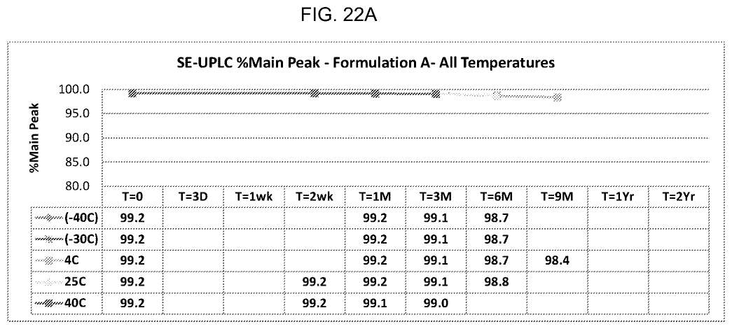

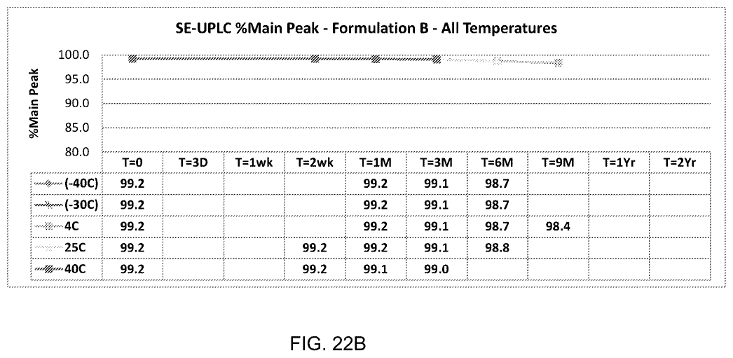

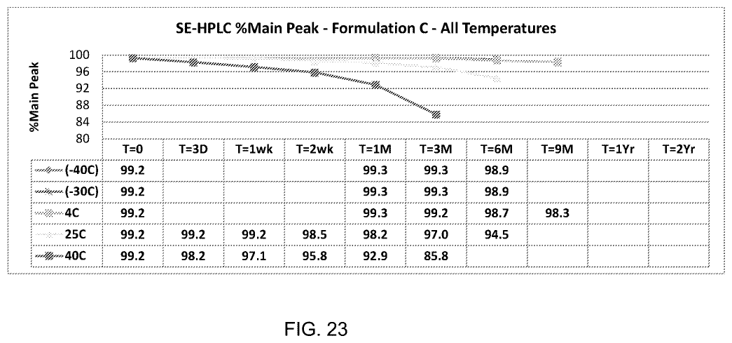

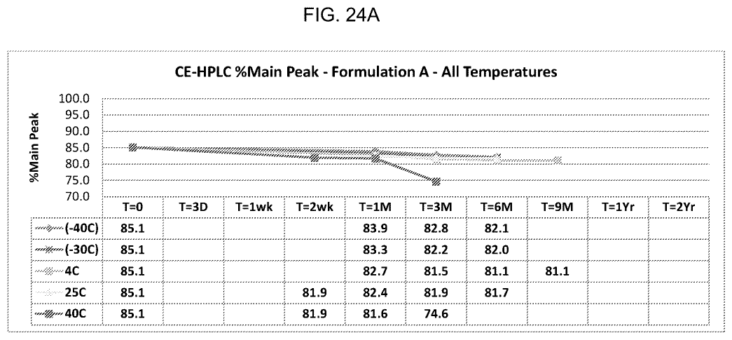

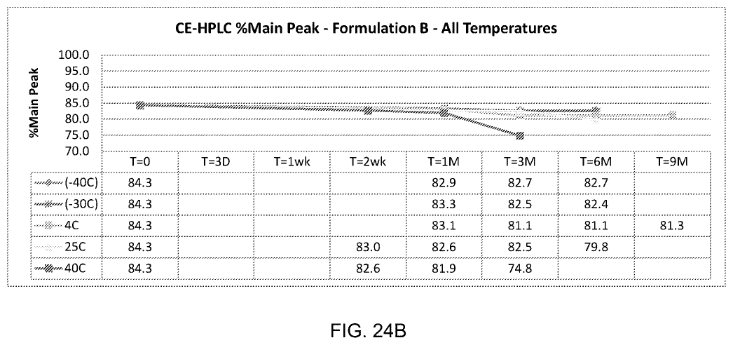

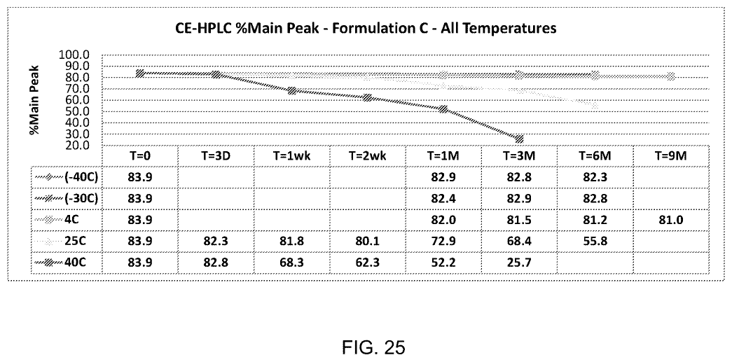

Antibody A was determined to have a 13.4% main peak loss via SE-UHPLC after 3 months at 40° C. when formulated in Formulation C. See FIG. 23. By contrast, Antibody A was determined to have a 0.2% and 0.0% main peak loss via SE-UHPLC under the same conditions when formulated in Formulation A (FIG. 22A) and Formulation B (FIG. 22B), respectively. Antibody A was determined to have a 58.2% main peak loss via CE-HPLC after 3 months at 40° C. when formulated in Formulation C. See FIG. 25. By contrast, Antibody A was determined to have a 10.5% and 0.5% main peak loss via CE-HPLC under the same conditions when formulated in Formulation A (FIG. 24A) and Formulation B (FIG. 24B), respectively.

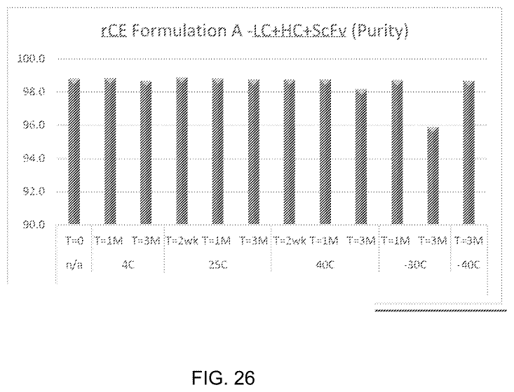

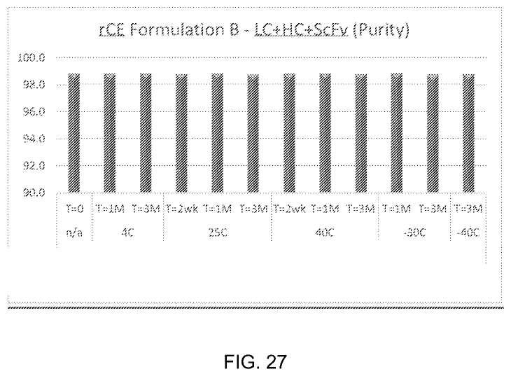

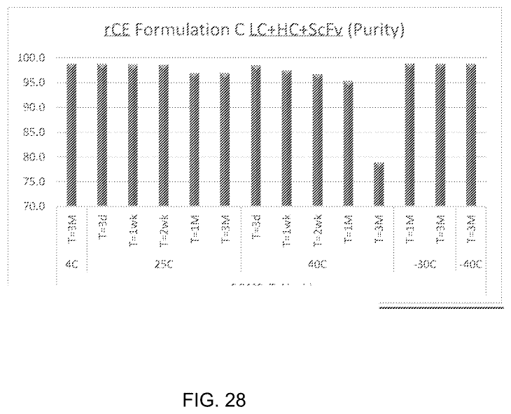

Antibody A showed a 2.9% main peak loss via rCE after three months at −30° C. when formulated in Formulation A (FIG. 26). Antibody A showed a 19.9% main peak loss via rCE after three months at 40° C. when formulated in Formulation C. See FIG. 28. However, Antibody A showed a 0.6% main peak loss via rCE after three months at 40° C. in Formulation A (FIG. 26), and a 0.0% main peak loss via rCE when formulated in Formulation B. See FIG. 27.

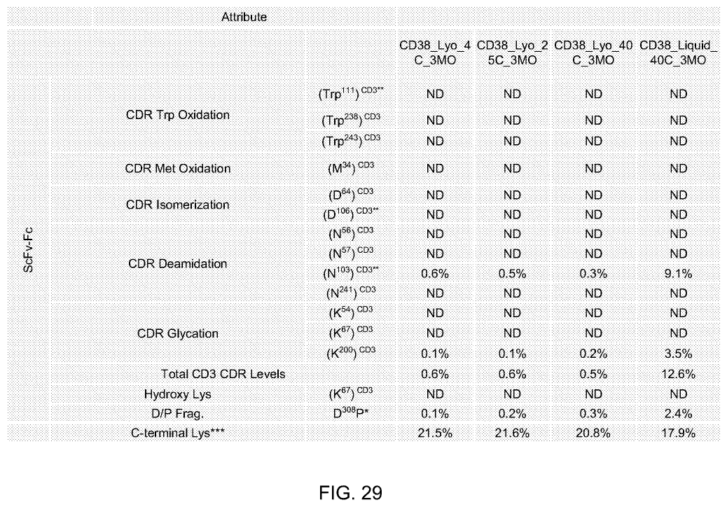

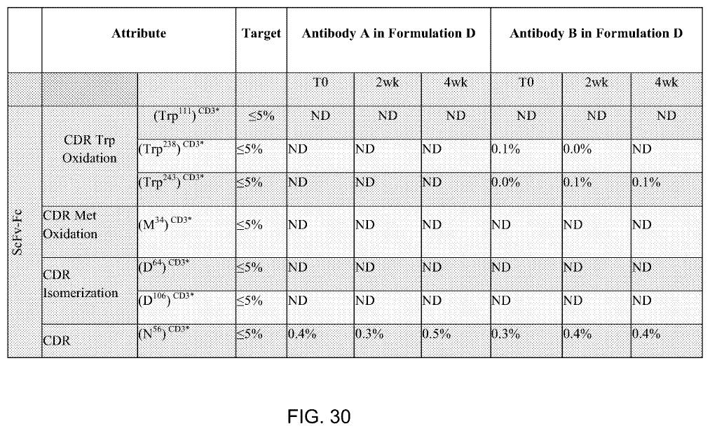

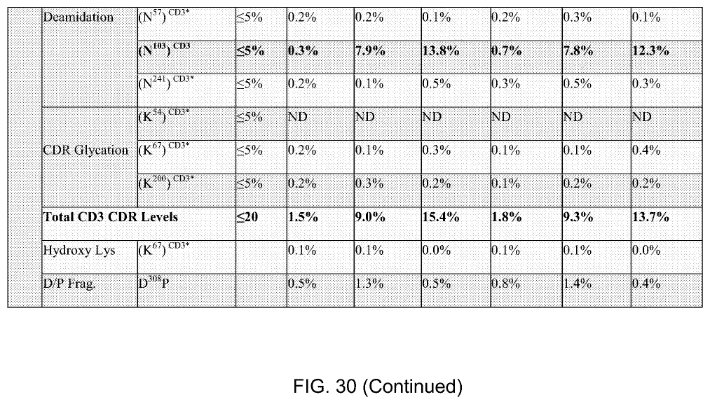

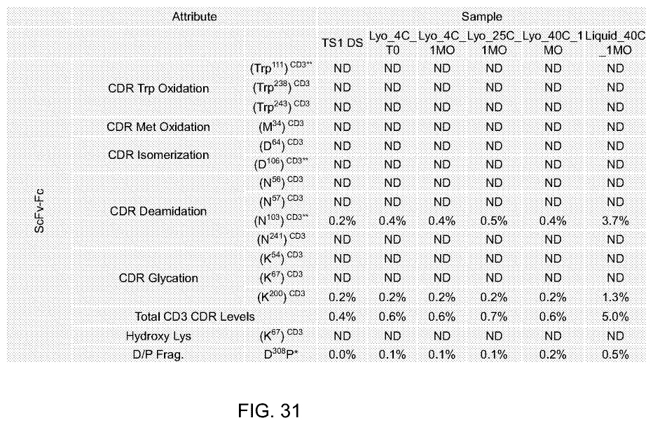

Antibody A showed 9.1% deamidation at N103 (CD3 scFv-FC) via MAM after three months at 40° C. when formulated in Formulation C. See FIG. 29, last column. By contrast, Antibody A showed 0.3% deamidation via MAM when formulated in Formulation B. See FIG. 29, second column from the right. Antibody A showed 13.8% deamidation at N103 (CD3 scFV-FC) via MAM after four weeks at 40° C. when formulated in Formulation D (see FIG. 30), and 3.7% deamidation at N103 (CD3 scFv-Fc) when formulated in Formulation C via MAM after one month at 40° C. See FIG. 31. By contrast, Antibody A showed 0.4% deamidation via MAM after one month at 40° C. when formulated in Formulation B. See FIG. 31.

The data provided herein demonstrates that that the lyophilized Formulation A and lyophilized Formulation B are more stable than the liquid Formulation C and liquid Formulation D due to high risk of deamidation in the liquid formulation.

### Example 2—Antibody Stability in Low pH Formulations

The following Example describes assays to verify the stability of a heterodimeric antibody described herein at various time points at various different temperatures (4° C., 25° C., 40° C., −30° C. & −40° C.) in liquid or lyophilized formulations at different protein concentrations (e.g., 1 mg/mL, 5 mg/mL and 20 mg/mL). Stability of the heterodimeric antibody was assessed in the following formulations:
Formulation E: 1 mg/mL heterodimeric antibody lyophilized formulation in 10 mM L-Glutamic acid, 9% (w/v) Sucrose, 0.01% (w/v) Polysorbate 80, pH4.2;
Formulation F: 5 mg/mL heterodimeric antibody lyophilized formulation in 10 mM L-Glutamic acid, 9% (w/v) Sucrose, 0.01% (w/v) Polysorbate 80, pH4.2;
Formulation G: 20 mg/mL heterodimeric antibody lyophilized formulation in 10 mM L-Glutamic acid, 9% (w/v) Sucrose, 0.01% (w/v) Polysorbate 80, pH4.2;
Formulation H: 1 mg/mL heterodimeric antibody liquid formulation in 10 mM L-Glutamic acid, 9% (w/v) Sucrose, 0.01% (w/v) Polysorbate 80, pH4.2;
Formulation I: 5 mg/mL heterodimeric antibody liquid formulation in 10 mM L-Glutamic acid, 9% (w/v) Sucrose, 0.01% (w/v) Polysorbate 80, pH4.2; and
Formulation J: 20 mg/mL heterodimeric antibody liquid formulation in 10 mM L-Glutamic acid, 9% (w/v) Sucrose, 0.01% (w/v) Polysorbate 80, pH4.2.

The stability of lyophilized Formulations E-G were compared to the stability of liquid liquid Formulations H-J.

The stability of the heterodimeric antibody was analyzed using the following assays: Appearance (via visual inspection of 20 vials at each time point), pH, Osmolality, CE-HPLC, rCE, MAM (Multi Attribute Method) and Karl Fischer (moisture content). The verification study samples were a 1.3 mL fill in 5cc vials. Formulations E-G were isotonic with an osmolality value of 314 mOsm/kg and 311 mOsm/kg, respectively. No proteinaceous particles were observed in any of the formulations tested.

Stability of the heterodimer antibody was assessed in the following formulations:

### Results:

Antibody A was determined to have a 34.7% main peak loss via CEX after 3 months at 40° C. when formulated in Formulation J. By contrast, Antibody A was determined to have a 2.8% main peak loss via CEX under the same conditions when formulated in Formulation G. See Table 2.

TABLE 2
% Main Peak of Formulations G and J as determined by CEX.

| Temperature | Time point | Formulation J | Formulation G |
|---|---|---|---|
| 40° C. | 0 | 74.8 | 74.4 |
| | 1 wk | 70.7 | 74.5 |
| | 2 wk | 67.4 | 73.6 |
| | 1 month | 68.4 | 74.8 |
| | 3 month | 40.1 | 71.6 |
| 25° C. | 0 | 74.8 | 74.4 |
| | 1 wk | 74.3 | N/A |
| | 2 wk | 74.0 | 75.5 |
| | 1 month | 73.2 | 75.2 |
| | 3 month | 68.7 | 74.3 |
| 4° C. | 0 | 74.8 | 74.4 |
| | 1 month | 74.9 | 75.1 |
| | 3 month | 75.2 | 73.8 |

Antibody A was determined to have a 15.3% main peak loss via SE-UHPLC after 3 months at 40° C. when formulated in Formulation J. By contrast, Antibody A was determined to have a 1.6% main peak loss via SE-UHPLC under the same conditions when formulated in Formulation G. See Table 3.

TABLE 3
% Main Peak of Formulations G and
J as determined by SE-UHPLC.

| Temperature | Time point | Formulation J | Formulation G |
|---|---|---|---|
| 40° C. | 0 | 98.6 | 98.6 |
| | 1 wk | 98.3 | 98.6 |
| | 2 wk | 98.4 | 99.1 |
| | 1 month | 97.4 | 99.1 |
| | 3 month | 83.3 | 97.0 |
| 25° C. | 0 | 98.6 | 98.6 |
| | 1 wk | 98.6 | N/A |
| | 2 wk | 98.9 | 99.0 |
| | 1 month | 99.8 | 99.1 |
| | 3 month | 95.9 | 97.0 |
| 4° C. | 0 | 98.6 | 98.6 |
| | 1 month | 99.1 | 99.1 |
| | 3 month | 97.0 | 97.1 |

Antibody A showed a 3.7% main peak loss via rCE after three months at 40° C. when formulated in Formulation J. However, Antibody A showed a 0.0% main peak loss via rCE after three months at 40° C. in Formulation G. See Table 4.

TABLE 4
% Main Peak of Formulations G and J as determined by rCE.

| Temperature | Time point | Formulation J | Formulation G |
|---|---|---|---|
| 40° C. | 0 | 99.7 | 99.7 |
| | 1 wk | 99.4 | 99.6 |
| | 1 month | 96.9 | 99.5 |
| | 3 month | 96.0 | 99.5 |
| 25° C. | 0 | 99.7 | 99.7 |
| | 1 wk | 99.6 | N/A |
| | 1 month | 99.2 | 99.5 |
| | 3 month | 98.8 | 99.5 |
| 4° C. | 0 | 99.7 | 99.7 |
| | 1 month | 99.5 | 99.5 |
| | 3 month | 99.4 | 99.4 |

Antibody A when formulated in Formulation G is expected to show the same % deamidation as Formulation B described above in Example 1.

The data provided herein demonstrates that the lyophilized Formulation G is more stable than the liquid Formulation J as demonstrated by a reduced % main peak loss observed in Formulation G at the tested time points. Also, it was determined that Formulation J, having a concentration of heterodimeric antibody concentration 20 mg/mL, was determined to have similar % main peak loss as Formulations A (heterodimeric antibody 1 mg/mL) and B (heterodimeric antibody 5 m/mL), which is surprising due to the higher heterodimeric antibody concentration present in Formulation J (20 mg/mL).

### Example 3—Antibody Stability in Low pH Formulations

The following Example describes assays to verify the stability of a heterodimeric antibody described herein for up to three years at various different temperatures (4° C., 25° C., 40° C., −30° C. & −40° C.) in liquid or lyophilized formulations at a protein concentration of 10 mg/mL. Stability of the heterodimeric antibody was assessed in the following formulations:
Formulation K: 10 mg/mL heterodimeric antibody lyophilized formulation in 10 mM L-Glutamic acid, 9% (w/v) Sucrose, 0.01% (w/v) Polysorbate 80, pH4.2; and
Formulation L: 10 mg/mL heterodimeric antibody liquid formulation in 10 mM L-Glutamic acid, 9% (w/v) Sucrose, 0.01% (w/v) Polysorbate 80, pH4.2.

The stability of lyophilized Formulation K was compared to the stability of liquid Formulation L Antibody B was utilized in this study.

The stability of the heterodimeric antibody was analyzed using the following assays: Appearance (via visual inspection of 20 vials at each time point), pH, Osmolality, SE-HPLC, rCE, MAM (Multi Attribute Method) and Karl Fischer (moisture content). The verification study samples were a 1.3 mL fill in 5cc vials. Formulation K was isotonic with an osmolality value of 305 mOsm/kg. No proteinaceous particles were observed in any of the formulations tested.

Stability of the heterodimer antibody was assessed in the following formulations:

### Results:

Antibody B was determined to have a 34.5% main peak loss via CEX after 3 months at 40° C. when formulated in Formulation L. By contrast, Antibody B was determined to have a 0.7% main peak loss via CEX under the same conditions when formulated in Formulation K. See Table 5.

TABLE 5
% Main Peak of Formulations K and L as determined by CEX.

| Temperature | Time point | Formulation L | Formulation K |
|---|---|---|---|
| 40° C. | 0 | 95.0 | 94.9 |
| | 1 wk | 91.6 | 94.6 |
| | 2 wk | 89.2 | 94.9 |
| | 1 month | 83.3 | 94.8 |
| | 3 months | 60.5 | 94.2 |
| 25° C. | 0 | 95.0 | 94.9 |
| | 1 wk | 94.7 | 94.7 |
| | 2 wk | 95.1 | 95.1 |
| | 1 month | 95.0 | 95.0 |
| | 3 months | 94.6 | 94.6 |
| 4° C. | 0 | 95.0 | 94.9 |
| | 1 wk | 94.7 | 94.8 |
| | 2 wk | 95.2 | 95.0 |
| | 1 month | 95.5 | 95.0 |
| | 3 months | 94.5 | 94.8 |
| −30° C. | 0 | 95.0 | 94.9 |
| | 1 wk | 94.8 | 94.7 |
| | 2 wk | 95.3 | 95.2 |
| | 1 month | 95.8 | 95.0 |
| | 3 months | 94.8 | 94.7 |

Antibody B was determined to have a 22.5% main peak loss via SE-HPLC after 3 months at 40° C. when formulated in Formulation L. By contrast, Antibody B was determined to have a 0.7% main peak loss via CEX under the same conditions when formulated in Formulation K. See Table 6 for SE-HPLC data.

TABLE 6
% Main Peak of Formulations K
and L as determined by SE-HPLC.

| Temperature | Time point | Formulation L | Formulation K |
|---|---|---|---|
| 40° C. | 0 | 98.8 | 98.8 |
| | 1 wk | 92.2 | 97.3 |
| | 2 wk | 92.6 | 98.5 |
| | 1 month | 91.7 | 98.7 |
| | 3 months | 76.3 | 97.1 |
| 25° C. | 0 | 98.8 | 98.8 |
| | 1 wk | 96.9 | 97.4 |
| | 2 wk | 96.7 | 98.6 |
| | 1 month | 98.7 | 98.7 |
| | 3 months | 97.2 | 97.2 |
| 4° C. | 0 | 98.8 | 98.8 |
| | 1 wk | 97.6 | 97.1 |
| | 2 wk | 97.9 | 98.6 |
| | 1 month | 98.5 | 98.7 |
| | 3 months | 96.7 | 97.0 |
| −30° C. | 0 | 98.8 | 98.8 |
| | 1 wk | 97.7 | 97.7 |
| | 2 wk | 98.0 | 98.0 |
| | 1 month | 98.7 | 98.7 |
| | 3 months | 96.8 | 97.5 |

Antibody B showed 6.8% deamidation via MAM after three months at 40° C. when formulated in Formulation L. See Table 7. By contrast, Antibody B showed <0.6% deamidation via MAM when formulated in Formulation K.

TABLE 7
% deamidation of Formulations K and L as determined by MAM.

| Temperature | Time point | Formulation L | Formulation K |
|---|---|---|---|
| 40° C. | 0 | <0.6% | <0.6% |
| | 1 month | 2.4% | <0.6% |
| | 3 months | 6.8% | <0.6% |
| 25° C. | 0 | <0.6% | <0.6% |
| | 1 month | <0.6% | <0.6% |
| | 3 months | 1.0% | <0.6% |
| 4° C. | 0 | <0.6% | <0.6% |
| | 1 month | <0.6% | <0.6% |
| | 3 months | <0.6% | <0.6% |
| −30° C. | 0 | <0.6% | <0.6% |
| | 1 month | <0.6% | <0.6% |
| | 3 months | <0.6% | <0.6% |

The data provided herein demonstrates that the lyophilized Formulation K is more stable than the liquid Formulation L due to high risk of deamidation in the liquid formulation.

1. A pharmaceutical composition comprising an antigen-binding protein, at least one buffer agent, at least one surfactant, and at least one saccharide, wherein the pH of the pharmaceutical composition ranges from 3.5 to 5.

2. The pharmaceutical composition of claim 1, wherein the antigen-binding protein is an antibody.

3. The pharmaceutical composition of claim 2, wherein the antibody is a heterodimeric antibody that binds CD3.

4-29. (canceled)

30. The pharmaceutical composition of claim 1, wherein the at least one buffer agent is an acid selected from the group consisting of acetate, glutamate, citrate, succinate, tartrate, fumarate, maleate, histidine, phosphate, and 2-(N-morpholino)ethanesulfonate or a combination thereof.

31. The pharmaceutical composition of claim 30, wherein the at least one buffer agent is present at a concentration range of 5 to 200 mM.

32. The pharmaceutical composition of claim 30, wherein the at least one buffer agent is present at a concentration range of 10 to 50 mM.

33. The pharmaceutical composition of claim 1, wherein the at least one saccharide is selected from the group consisting of monosaccharide, disaccharide, cyclic polysaccharide, sugar alcohol, linear branched dextran, and linear non-branched dextran.

34. The pharmaceutical composition of claim 33, wherein the disaccharide is selected from the group consisting of sucrose, trehalose, mannitol, and sorbitol or a combination thereof.

35. The pharmaceutical composition of claim 33, wherein the sugar alcohol is sorbitol.

36. The pharmaceutical composition of claim 1, wherein the at least one saccharide is present at a concentration in the range of 1 to 15% (w/V).

37. The pharmaceutical composition of claim 1, wherein the at least one saccharide is present at a concentration in the range of 5 to 12% (w/V).

38. The pharmaceutical composition of claim 1, wherein the at least one saccharide is present at a concentration in the range of 7 to 12% (w/V).

39. The pharmaceutical composition of claim 38, wherein the at least one saccharide is present at a concentration in the range of 9 to 12% (w/V).

40. The pharmaceutical composition of claim 1, wherein the at least one surfactant is selected from the group consisting of polysorbate 20, polysorbate 40, polysorbate 60, polysorbate 80, poloxamer 188, pluronic F68, triton X-100, polyoxyethylen3, and PEG 3350, PEG 4000, or a combination thereof.

41. The pharmaceutical composition of claim 1, wherein the at least one surfactant is present at a concentration in the range of 0.001 to 0.5% (w/V).

42-44. (canceled)

45. The pharmaceutical composition of claim 1, wherein the pH of the composition ranges from 4.0 to 5.0.

46-49. (canceled)

50. The pharmaceutical composition of claim 1, wherein the composition comprises 10 mM glutamate, 9% (w/V) sucrose and 0.01% (w/V) polysorbate 80, and wherein the pH of the liquid pharmaceutical composition is 4.2.

51. The pharmaceutical composition of claim 3, wherein the heterodimeric antibody is present in a concentration range of 0.1 to 8 mg/ml.

52-59. (canceled)

60. A method of treating cancer in a subject in need thereof comprising administering the composition of claim 1 to the subject.

61-62. (canceled)


SEQUENCE LISTING (391 sequences)

SEQ ID NO: 1 - protein, 125 aa, synthetic construct
  REGION 1..125: note = Variable heavy (vh) domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 2 - protein, 5 aa, synthetic construct
  REGION 1..5: note = vhCDR1
TYAMN

SEQ ID NO: 3 - protein, 19 aa, synthetic construct
  REGION 1..19: note = vhCDR2
RIRSKYNNYATYYADSVKG

SEQ ID NO: 4 - protein, 14 aa, synthetic construct
  REGION 1..14: note = vhCDR3
HGNFGDSYVSWFAY

SEQ ID NO: 5 - protein, 109 aa, synthetic construct
  REGION 1..109: note = Variable light (vl) domain
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 6 - protein, 14 aa, synthetic construct
  REGION 1..14: note = vlCDR1
GSSTGAVTTSNYAN

SEQ ID NO: 7 - protein, 7 aa, synthetic construct
  REGION 1..7: note = vlCDR2
GTNKRAP

SEQ ID NO: 8 - protein, 9 aa, synthetic construct
  REGION 1..9: note = vlCDR3
ALWYSNHWV

SEQ ID NO: 9 - protein, 254 aa, synthetic construct
  REGION 1..254: note = scFv (including charged linker)
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 10 - protein, 125 aa, synthetic construct
  REGION 1..125: note = Variable heavy (vh) domain
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSS

SEQ ID NO: 11 - protein, 5 aa, synthetic construct
  REGION 1..5: note = vhCDR1
TYAMN

SEQ ID NO: 12 - protein, 19 aa, synthetic construct
  REGION 1..19: note = vhCDR2
RIRSKANNYATYYADSVKG

SEQ ID NO: 13 - protein, 14 aa, synthetic construct
  REGION 1..14: note = vhCDR3
HGNFGDSYVSWFAY

SEQ ID NO: 14 - protein, 109 aa, synthetic construct; identical to SEQ ID NO: 5
  REGION 1..109: note = Variable light (vl) domain

SEQ ID NO: 15 - protein, 14 aa, synthetic construct
  REGION 1..14: note = vlCDR1
GSSTGAVTTSNYAN

SEQ ID NO: 16 - protein, 7 aa, synthetic construct
  REGION 1..7: note = vlCDR2
GTNKRAP

SEQ ID NO: 17 - protein, 9 aa, synthetic construct
  REGION 1..9: note = vlCDR3
ALWYSNHWV

SEQ ID NO: 18 - protein, 254 aa, synthetic construct
  REGION 1..254: note = scFv (including charged linker)
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 19 - protein, 125 aa, synthetic construct
  REGION 1..125: note = Variable heavy (vh) domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSS

SEQ ID NO: 20 - protein, 5 aa, synthetic construct
  REGION 1..5: note = vhCDR1
TYAMN

SEQ ID NO: 21 - protein, 19 aa, synthetic construct
  REGION 1..19: note = vhCDR2
RIRSKYNNYATYYADSVKG

SEQ ID NO: 22 - protein, 14 aa, synthetic construct
  REGION 1..14: note = vhCDR3
HGNFGDEYVSWFAY

SEQ ID NO: 23 - protein, 109 aa, synthetic construct; identical to SEQ ID NO: 5
  REGION 1..109: note = Variable light (vl) domain

SEQ ID NO: 24 - protein, 14 aa, synthetic construct
  REGION 1..14: note = vlCDR1
GSSTGAVTTSNYAN

SEQ ID NO: 25 - protein, 7 aa, synthetic construct
  REGION 1..7: note = vlCDR2
GTNKRAP

SEQ ID NO: 26 - protein, 9 aa, synthetic construct
  REGION 1..9: note = vlCDR3
ALWYSNHWV

SEQ ID NO: 27 - protein, 254 aa, synthetic construct
  REGION 1..254: note = scFv (including charged linker)
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 28 - protein, 125 aa, synthetic construct
  REGION 1..125: note = Variable heavy (vh) domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDPYVSWFAYWGQGTLVTVSS

SEQ ID NO: 29 - protein, 5 aa, synthetic construct
  REGION 1..5: note = vhCDR1
TYAMN

SEQ ID NO: 30 - protein, 19 aa, synthetic construct
  REGION 1..19: note = vhCDR2
RIRSKYNNYATYYADSVKG

SEQ ID NO: 31 - protein, 14 aa, synthetic construct
  REGION 1..14: note = vhCDR3
HGNFGDPYVSWFAY

SEQ ID NO: 32 - protein, 109 aa, synthetic construct; identical to SEQ ID NO: 5
  REGION 1..109: note = Variable light (vl) domain

SEQ ID NO: 33 - protein, 14 aa, synthetic construct
  REGION 1..14: note = vlCDR1
GSSTGAVTTSNYAN

SEQ ID NO: 34 - protein, 7 aa, synthetic construct
  REGION 1..7: note = vlCDR2
GTNKRAP

SEQ ID NO: 35 - protein, 9 aa, synthetic construct
  REGION 1..9: note = vlCDR3
ALWYSNHWV

SEQ ID NO: 36 - protein, 254 aa, synthetic construct
  REGION 1..254: note = scFv (including charged linker)
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDPYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 37 - protein, 125 aa, synthetic construct
  REGION 1..125: note = Variable heavy (vh) domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSS

SEQ ID NO: 38 - protein, 5 aa, synthetic construct
  REGION 1..5: note = vhCDR1
TYAMN

SEQ ID NO: 39 - protein, 19 aa, synthetic construct
  REGION 1..19: note = vhCDR2
RIRSKYNNYATYYADSVKG

SEQ ID NO: 40 - protein, 14 aa, synthetic construct
  REGION 1..14: note = vhCDR3
HGNFGDSYVSWFDY

SEQ ID NO: 41 - protein, 109 aa, synthetic construct; identical to SEQ ID NO: 5
  REGION 1..109: note = Variable light (vl) domain

SEQ ID NO: 42 - protein, 14 aa, synthetic construct
  REGION 1..14: note = vlCDR1
GSSTGAVTTSNYAN

SEQ ID NO: 43 - protein, 7 aa, synthetic construct
  REGION 1..7: note = vlCDR2
GTNKRAP

SEQ ID NO: 44 - protein, 9 aa, synthetic construct
  REGION 1..9: note = vlCDR3
ALWYSNHWV

SEQ ID NO: 45 - protein, 254 aa, synthetic construct
  REGION 1..254: note = scFv (including charged linker)
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 46 - protein, 125 aa, synthetic construct
  REGION 1..125: note = Variable heavy (vh) domain
EVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 47 - protein, 5 aa, synthetic construct
  REGION 1..5: note = vhCDR1
TYAMS

SEQ ID NO: 48 - protein, 19 aa, synthetic construct
  REGION 1..19: note = vhCDR2
RIRSKYNNYATYYADSVKG

SEQ ID NO: 49 - protein, 14 aa, synthetic construct
  REGION 1..14: note = vhCDR3
HGNFGDSYVSWFAY

SEQ ID NO: 50 - protein, 109 aa, synthetic construct; identical to SEQ ID NO: 5
  REGION 1..109: note = Variable light (vl) domain

SEQ ID NO: 51 - protein, 14 aa, synthetic construct
  REGION 1..14: note = vlCDR1
GSSTGAVTTSNYAN

SEQ ID NO: 52 - protein, 7 aa, synthetic construct
  REGION 1..7: note = vlCDR2
GTNKRAP

SEQ ID NO: 53 - protein, 9 aa, synthetic construct
  REGION 1..9: note = vlCDR3
ALWYSNHWV

SEQ ID NO: 54 - protein, 254 aa, synthetic construct
  REGION 1..254: note = scFv (including charged linker)
EVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 55 - protein, 116 aa, synthetic construct
  REGION 1..116: note = Variable heavy (vh) domain
EVQLVESGGGLVQPGGSLRLSCAASGFTFSYSWMNWVRQAPGKGLEWVSEINPQSSTINYATSVKGRFTISRDNSKNTLYLQMNSLRAEDTAVYYCARYGNWFPYWGQGTLVTVSS

SEQ ID NO: 56 - protein, 5 aa, synthetic construct
  REGION 1..5: note = vhCDR1
YSWMN

SEQ ID NO: 57 - protein, 17 aa, synthetic construct
  REGION 1..17: note = vhCDR2
EINPQSSTINYATSVKG

SEQ ID NO: 58 - protein, 7 aa, synthetic construct
  REGION 1..7: note = vhCDR3
YGNWFPY

SEQ ID NO: 59 - protein, 107 aa, synthetic construct
  REGION 1..107: note = Variable light (vl) domain
DIVMTQSPSSLSASVGDRVTITCRASQNVDTWVAWYQQKPGQSPKALIYSASYRYSGVPDRFTGSGSGTDFTLTISSLQPEDFATYFCQQYDSYPLTFGGGTKLEIK

SEQ ID NO: 60 - protein, 11 aa, synthetic construct
  REGION 1..11: note = vlCDR1
RASQNVDTWVA

SEQ ID NO: 61 - protein, 7 aa, synthetic construct
  REGION 1..7: note = vlCDR2
SASYRYS

SEQ ID NO: 62 - protein, 9 aa, synthetic construct
  REGION 1..9: note = vlCDR3
QQYDSYPLT

SEQ ID NO: 63 - protein, 243 aa, synthetic construct
  REGION 1..243: note = scFv (including charged linker)
EVQLVESGGGLVQPGGSLRLSCAASGFTFSYSWMNWVRQAPGKGLEWVSEINPQSSTINYATSVKGRFTISRDNSKNTLYLQMNSLRAEDTAVYYCARYGNWFPYWGQGTLVTVSSGKPGSGKPGSGKPGSGKPGSDIVMTQSPSSLSASVGDRVTITCRASQNVDTWVAWYQQKPGQSPKALIYSASYRYSGVPDRFTGSGSGTDFTLTISSLQPEDFATYFCQQYDSYPLTFGGGTKLEIK

SEQ ID NO: 64 - protein, 116 aa, synthetic construct
  REGION 1..116: note = Variable heavy (vh) domain
EVQLVESGGGLVQPGGSLRLSCAASGFDFSRSWMNWVRQAPGKGLEWVSEINPDSSTINYATSVKGRFTISRDNSKNTLYLQMNSLRAEDTAVYYCARYGNWFPYWGQGTLVTVSS

SEQ ID NO: 65 - protein, 5 aa, synthetic construct
  REGION 1..5: note = vhCDR1
RSWMN

SEQ ID NO: 66 - protein, 17 aa, synthetic construct
  REGION 1..17: note = vhCDR2
EINPDSSTINYATSVKG

SEQ ID NO: 67 - protein, 7 aa, synthetic construct
  REGION 1..7: note = vhCDR3
YGNWFPY

SEQ ID NO: 68 - protein, 107 aa, synthetic construct; identical to SEQ ID NO: 59
  REGION 1..107: note = Variable light (vl) domain

SEQ ID NO: 69 - protein, 11 aa, synthetic construct
  REGION 1..11: note = vlCDR1
RASQNVDTWVA

SEQ ID NO: 70 - protein, 7 aa, synthetic construct
  REGION 1..7: note = vlCDR2
SASYRYS

SEQ ID NO: 71 - protein, 9 aa, synthetic construct
  REGION 1..9: note = vlCDR3
QQYDSYPLT

SEQ ID NO: 72 - protein, 243 aa, synthetic construct
  REGION 1..243: note = scFv (including charged linker)
EVQLVESGGGLVQPGGSLRLSCAASGFDFSRSWMNWVRQAPGKGLEWVSEINPDSSTINYATSVKGRFTISRDNSKNTLYLQMNSLRAEDTAVYYCARYGNWFPYWGQGTLVTVSSGKPGSGKPGSGKPGSGKPGSDIVMTQSPSSLSASVGDRVTITCRASQNVDTWVAWYQQKPGQSPKALIYSASYRYSGVPDRFTGSGSGTDFTLTISSLQPEDFATYFCQQYDSYPLTFGGGTKLEIK

SEQ ID NO: 73 - protein, 116 aa, synthetic construct; identical to SEQ ID NO: 64
  REGION 1..116: note = Variable heavy (vh) domain

SEQ ID NO: 74 - protein, 5 aa, synthetic construct
  REGION 1..5: note = vhCDR1
RSWMN

SEQ ID NO: 75 - protein, 17 aa, synthetic construct
  REGION 1..17: note = vhCDR2
EINPDSSTINYATSVKG

SEQ ID NO: 76 - protein, 7 aa, synthetic construct
  REGION 1..7: note = vhCDR3
YGNWFPY

SEQ ID NO: 77 - protein, 116 aa, synthetic construct; identical to SEQ ID NO: 64
  REGION 1..116: note = Variable light (vl) domain

SEQ ID NO: 78 - protein, 11 aa, synthetic construct
  REGION 1..11: note = vlCDR1
RASQNVDTNVA

SEQ ID NO: 79 - protein, 7 aa, synthetic construct
  REGION 1..7: note = vlCDR2
SASYRYS

SEQ ID NO: 80 - protein, 9 aa, synthetic construct
  REGION 1..9: note = vlCDR3
QQYDSYPLT

SEQ ID NO: 81 - protein, 252 aa, synthetic construct
  REGION 1..252: note = scFv (including charged linker)
EVQLVESGGGLVQPGGSLRLSCAASGFDFSRSWMNWVRQAPGKGLEWVSEINPDSSTINYATSVKGRFTISRDNSKNTLYLQMNSLRAEDTAVYYCARYGNWFPYWGQGTLVTVSSGKPGSGKPGSGKPGSGKPGSEVQLVESGGGLVQPGGSLRLSCAASGFDFSRSWMNWVRQAPGKGLEWVSEINPDSSTINYATSVKGRFTISRDNSKNTLYLQMNSLRAEDTAVYYCARYGNWFPYWGQGTLVTVSS

SEQ ID NO: 82 - protein, 445 aa, synthetic construct
  REGION 1..445: note = XENP13551 HC-Fab
EVQLVESGGGLVQPGGSLRLSCAASGFTFSYSWMNWVRQAPGKGLEWVSEINPQSSTINYATSVKGRFTISRDNSKNTLYLQMNSLRAEDTAVYYCARYGNWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 83 - protein, 485 aa, synthetic construct
  REGION 1..485: note = XENP13551 HC-scFv
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 214 aa, synthetic construct
  REGION 1..214: note = XENP13551 LC
DIVMTQSPSSLSASVGDRVTITCRASQNVDTWVAWYQQKPGQSPKALIYSASYRYSGVPDRFTGSGSGTDFTLTISSLQPEDFATYFCQQYDSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 445 aa, synthetic construct
  REGION 1..445: note = XENP13243 HC-Fab
EVQLVESGGGLVQPGGSLRLSCAASGFDFSRSWMNWVRQAPGKGLEWVSEINPDSSTINYATSVKGRFTISRDNSKNTLYLQMNSLRAEDTAVYYCARYGNWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 86 - protein, 485 aa, synthetic construct; identical to SEQ ID NO: 83
  REGION 1..485: note = XENP13243 HC-scFv

SEQ ID NO: 87 - protein, 214 aa, synthetic construct
  REGION 1..214: note = XENP13243 LC
DIVMTQSPSSLSASVGDRVTITCRASQNVDTNVAWYQQKPGQSPKALIYSASYRYSGVPDRFTGSGSGTDFTLTISSLQPEDFATYFCQQYDSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 445 aa, synthetic construct; identical to SEQ ID NO: 82
  REGION 1..445: note = XENP14702 Anti-CD38

SEQ ID NO: 89 - protein, 485 aa, synthetic construct
  REGION 1..485: note = XENP14702 Anti-CD38
EVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 214 aa, synthetic construct; identical to SEQ ID NO: 84
  REGION 1..214: note = XENP14702 Anti-CD38

SEQ ID NO: 91 - protein, 445 aa, synthetic construct; identical to SEQ ID NO: 82
  REGION 1..445: note = XENP15426 Anti-CD38

SEQ ID NO: 92 - protein, 485 aa, synthetic construct
  REGION 1..485: note = XENP15426 Anti-CD38
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 214 aa, synthetic construct; identical to SEQ ID NO: 84
  REGION 1..214: note = XENP15426 Anti-CD38

SEQ ID NO: 94 - protein, 445 aa, synthetic construct; identical to SEQ ID NO: 85
  REGION 1..445: note = XENP14701 Anti-CD38

SEQ ID NO: 95 - protein, 485 aa, synthetic construct; identical to SEQ ID NO: 89
  REGION 1..485: note = XENP14701 Anti-CD38

SEQ ID NO: 96 - protein, 214 aa, synthetic construct; identical to SEQ ID NO: 87
  REGION 1..214: note = XENP14701 Anti-CD38

SEQ ID NO: 97 - protein, 445 aa, synthetic construct; identical to SEQ ID NO: 85
  REGION 1..445: note = XENP14703 Heavy Chain 1

SEQ ID NO: 98 - protein, 485 aa, synthetic construct; identical to SEQ ID NO: 89
  REGION 1..485: note = XENP14703 Heavy Chain 2

SEQ ID NO: 99 - protein, 214 aa, synthetic construct
  REGION 1..214: note = XENP14703 Light Chain
DIVMTQSPSSLSASVGDRVTITCRASQNVDTNVAWYQQKPGQSPKALIYSASYRKSGVPDRFTGSGSGTDFTLTISSLQPEDFATYFCQQYDSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 445 aa, synthetic construct; identical to SEQ ID NO: 85
  REGION 1..445: note = XENP13243 Anti-CD38

SEQ ID NO: 101 - protein, 485 aa, synthetic construct; identical to SEQ ID NO: 83
  REGION 1..485: note = XENP13243 Anti-CD38

SEQ ID NO: 102 - protein, 214 aa, synthetic construct; identical to SEQ ID NO: 87
  REGION 1..214: note = XENP13243 Anti-CD38

SEQ ID NO: 103 - protein, 445 aa, synthetic construct; identical to SEQ ID NO: 82
  REGION 1..445: note = XENP18967 Anti-CD38

SEQ ID NO: 104 - protein, 485 aa, synthetic construct
  REGION 1..485: note = XENP18967 Anti-CD38
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 214 aa, synthetic construct; identical to SEQ ID NO: 84
  REGION 1..214: note = XENP18967 Anti-CD38

SEQ ID NO: 106 - protein, 445 aa, synthetic construct; identical to SEQ ID NO: 85
  REGION 1..445: note = XENP18971 Anti-CD38

SEQ ID NO: 107 - protein, 485 aa, synthetic construct; identical to SEQ ID NO: 104
  REGION 1..485: note = XENP18971 Anti-CD38

SEQ ID NO: 108 - protein, 214 aa, synthetic construct; identical to SEQ ID NO: 87
  REGION 1..214: note = XENP18971 Anti-CD38

SEQ ID NO: 109 - protein, 445 aa, synthetic construct; identical to SEQ ID NO: 85
  REGION 1..445: note = XENP18969 Anti-CD38

SEQ ID NO: 110 - protein, 485 aa, synthetic construct; identical to SEQ ID NO: 92
  REGION 1..485: note = XENP18969 Anti-CD38

SEQ ID NO: 111 - protein, 214 aa, synthetic construct; identical to SEQ ID NO: 84
  REGION 1..214: note = XENP18969 Anti-CD38

SEQ ID NO: 112 - protein, 445 aa, synthetic construct; identical to SEQ ID NO: 85
  REGION 1..445: note = XENP18970 Anti-CD38

SEQ ID NO: 113 - protein, 485 aa, synthetic construct; identical to SEQ ID NO: 89
  REGION 1..485: note = XENP18970 Anti-CD38

SEQ ID NO: 114 - protein, 214 aa, synthetic construct; identical to SEQ ID NO: 84
  REGION 1..214: note = XENP18970 Anti-CD38

SEQ ID NO: 115 - protein, 445 aa, synthetic construct; identical to SEQ ID NO: 85
  REGION 1..445: note = XENP18972 Anti-CD38

SEQ ID NO: 116 - protein, 485 aa, synthetic construct; identical to SEQ ID NO: 92
  REGION 1..485: note = XENP18972 Anti-CD38

SEQ ID NO: 117 - protein, 214 aa, synthetic construct; identical to SEQ ID NO: 87
  REGION 1..214: note = XENP18972 Anti-CD38

SEQ ID NO: 118 - protein, 445 aa, synthetic construct; identical to SEQ ID NO: 85
  REGION 1..445: note = XENP18973 Anti-CD38

SEQ ID NO: 119 - protein, 485 aa, synthetic construct; identical to SEQ ID NO: 89
  REGION 1..485: note = XENP18973 Anti-CD38

SEQ ID NO: 120 - protein, 214 aa, synthetic construct; identical to SEQ ID NO: 87
  REGION 1..214: note = XENP18973 Anti-CD38

SEQ ID NO: 121 - protein, 445 aa, synthetic construct; identical to SEQ ID NO: 85
  REGION 1..445: note = XENP15055 Anti-CD38

SEQ ID NO: 122 - protein, 485 aa, synthetic construct; identical to SEQ ID NO: 92
  REGION 1..485: note = XENP15055 Anti-CD38

SEQ ID NO: 123 - protein, 214 aa, synthetic construct; identical to SEQ ID NO: 87
  REGION 1..214: note = XENP15055 Anti-CD38

SEQ ID NO: 124 - protein, 445 aa, synthetic construct; identical to SEQ ID NO: 85
  REGION 1..445: note = XENP13544 Anti-CD38

SEQ ID NO: 125 - protein, 485 aa, synthetic construct
  REGION 1..485: note = XENP13544 Anti-CD38
EVQLVESGGGLVQPGGSLRLSCAASGFTFSTYAMNWVRQAPGKCLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGCGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 214 aa, synthetic construct; identical to SEQ ID NO: 87
  REGION 1..214: note = XENP13544 Anti-CD38

SEQ ID NO: 127 - protein, 445 aa, synthetic construct; identical to SEQ ID NO: 82
  REGION 1..445: note = XENP13694 Anti-CD38

SEQ ID NO: 128 - protein, 485 aa, synthetic construct; identical to SEQ ID NO: 125
  REGION 1..485: note = XENP13694 Anti-CD38

SEQ ID NO: 129 - protein, 214 aa, synthetic construct; identical to SEQ ID NO: 84
  REGION 1..214: note = XENP13694 Anti-CD38

SEQ ID NO: 130 - protein, 207 aa, synthetic construct
  REGION 1..207: note = CD3E HUMAN Tcell
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 131 - protein, 300 aa, synthetic construct
  REGION 1..300: note = Human CD38 sequence
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 132 - protein, 258 aa, synthetic construct
  REGION 1..258: note = ECD domain
VPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 133 - protein, 15 aa, synthetic construct
  REGION 1..15: note = Positive charged scFv linkers
GGGGSGGGGSGGGGS

SEQ ID NO: 134 - protein, 18 aa, synthetic construct
  REGION 1..18: note = Positive charged scFv linkers
GSTSGSGKPGSGEGSTKG

SEQ ID NO: 135 - protein, 14 aa, synthetic construct
  REGION 1..14: note = Positive charged scFv linkers
IRPRAIGGSKPRVA

SEQ ID NO: 136 - protein, 15 aa, synthetic construct
  REGION 1..15: note = Positive charged scFv linkers
GKGGSGKGGSGKGGS

SEQ ID NO: 137 - protein, 15 aa, synthetic construct
  REGION 1..15: note = Positive charged scFv linkers
GGKGSGGKGSGGKGS

SEQ ID NO: 138 - protein, 15 aa, synthetic construct
  REGION 1..15: note = Positive charged scFv linkers
GGGKSGGGKSGGGKS

SEQ ID NO: 139 - protein, 15 aa, synthetic construct
  REGION 1..15: note = Positive charged scFv linkers
GKGKSGKGKSGKGKS

SEQ ID NO: 140 - protein, 15 aa, synthetic construct
  REGION 1..15: note = Positive charged scFv linkers
GGGKSGGKGSGKGGS

SEQ ID NO: 141 - protein, 15 aa, synthetic construct
  REGION 1..15: note = Positive charged scFv linkers
GKPGSGKPGSGKPGS

SEQ ID NO: 142 - protein, 20 aa, synthetic construct
  REGION 1..20: note = Positive charged scFv linkers
GKPGSGKPGSGKPGSGKPGS

SEQ ID NO: 143 - protein, 20 aa, synthetic construct
  REGION 1..20: note = Positive charged scFv linkers
GKGKSGKGKSGKGKSGKGKS

SEQ ID NO: 144 - protein, 20 aa, synthetic construct
  REGION 1..20: note = Negative charged scFv linkers
GGGGSGGGGSGGGGSGGGGS

SEQ ID NO: 145 - protein, 14 aa, synthetic construct
  REGION 1..14: note = Negative charged scFv linkers
STAGDTHLGGEDFD

SEQ ID NO: 146 - protein, 15 aa, synthetic construct
  REGION 1..15: note = Negative charged scFv linkers
GEGGSGEGGSGEGGS

SEQ ID NO: 147 - protein, 15 aa, synthetic construct
  REGION 1..15: note = Negative charged scFv linkers
GGEGSGGEGSGGEGS

SEQ ID NO: 148 - protein, 15 aa, synthetic construct
  REGION 1..15: note = Negative charged scFv linkers
GGGESGGGESGGGES

SEQ ID NO: 149 - protein, 15 aa, synthetic construct
  REGION 1..15: note = Negative charged scFv linkers
GEGESGEGESGEGES

SEQ ID NO: 150 - protein, 15 aa, synthetic construct
  REGION 1..15: note = Negative charged scFv linkers
GGGESGGEGSGEGGS

SEQ ID NO: 151 - protein, 20 aa, synthetic construct
  REGION 1..20: note = Negative charged scFv linkers
GEGESGEGESGEGESGEGES

SEQ ID NO: 152 - protein, 15 aa, synthetic construct
  REGION 1..15: note = scFv Linkers
GGGGSGGGGSGGGGS

SEQ ID NO: 153 - protein, 20 aa, synthetic construct
  REGION 1..20: note = scFv Linkers
GGGGSGGGGSGGGGSGGGGS

SEQ ID NO: 154 - protein, 18 aa, synthetic construct
  REGION 1..18: note = scFv Linkers
GSTSGSGKPGSGEGSTKG

SEQ ID NO: 155 - protein, 19 aa, synthetic construct
  REGION 1..19: note = scFv Linkers
PRGASKSGSASQTGSAPGS

SEQ ID NO: 156 - protein, 19 aa, synthetic construct
  REGION 1..19: note = scFv Linkers
GTAAAGAGAAGGAAAGAAG

SEQ ID NO: 157 - protein, 19 aa, synthetic construct
  REGION 1..19: note = scFv Linkers
GTSGSSGSGSGGSGSGGGG

SEQ ID NO: 158 - protein, 20 aa, synthetic construct
  REGION 1..20: note = scFv Linkers
GKPGSGKPGSGKPGSGKPGS

SEQ ID NO: 159 - protein, 445 aa, synthetic construct; identical to SEQ ID NO: 85
  REGION 1..445: note = XENP14419 Anti-CD38

SEQ ID NO: 160 - protein, 714 aa, synthetic construct
  REGION 1..714: note = XENP14419 Anti-CD38
EVQLVESGGGLVQPGGSLRLSCAASGFDFSRSWMNWVRQAPGKGLEWVSEINPDSSTINYATSVKGRFTISRDNSKNTLYLQMNSLRAEDTAVYYCARYGNWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 161 - protein, 214 aa, synthetic construct; identical to SEQ ID NO: 87
  REGION 1..214: note = XENP14419 Anti-CD38

SEQ ID NO: 162 - protein, 585 aa, synthetic construct
  REGION 1..585: note = XENP14420 Anti-CD38
EVQLVESGGGLVQPGGSLRLSCAASGFDFSRSWMNWVRQAPGKGLEWVSEINPDSSTINYATSVKGRFTISRDNSKNTLYLQMNSLRAEDTAVYYCARYGNWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGKGGGGSGGGGSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 163 - protein, 569 aa, synthetic construct
  REGION 1..569: note = XENP14420 Anti-CD38
EVQLVESGGGLVQPGGSLRLSCAASGFDFSRSWMNWVRQAPGKGLEWVSEINPDSSTINYATSVKGRFTISRDNSKNTLYLQMNSLRAEDTAVYYCARYGNWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 164 - protein, 214 aa, synthetic construct; identical to SEQ ID NO: 87
  REGION 1..214: note = XENP14420 Anti-CD38

SEQ ID NO: 165 - protein, 445 aa, synthetic construct; identical to SEQ ID NO: 85
  REGION 1..445: note = XENP14421 Anti-CD38

SEQ ID NO: 166 - protein, 718 aa, synthetic construct
  REGION 1..718: note = XENP14421 Anti-CD38
EVQLVESGGGLVQPGGSLRLSCAASGFDFSRSWMNWVRQAPGKGLEWVSEINPDSSTINYATSVKGRFTISRDNSKNTLYLQMNSLRAEDTAVYYCARYGNWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 214 aa, synthetic construct; identical to SEQ ID NO: 87
  REGION 1..214: note = XENP14421 Anti-CD38

SEQ ID NO: 168 - protein, 231 aa, synthetic construct
  REGION 1..231: note = XENP14422 Anti-CD38
EPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 169 - protein, 718 aa, synthetic construct; identical to SEQ ID NO: 166
  REGION 1..718: note = XENP14422 Anti-CD38

SEQ ID NO: 170 - protein, 214 aa, synthetic construct; identical to SEQ ID NO: 87
  REGION 1..214: note = XENP14422 Anti-CD38

SEQ ID NO: 171 - protein, 589 aa, synthetic construct
  REGION 1..589: note = XENP14423 Anti-CD38
EVQLVESGGGLVQPGGSLRLSCAASGFDFSRSWMNWVRQAPGKGLEWVSEINPDSSTINYATSVKGRFTISRDNSKNTLYLQMNSLRAEDTAVYYCARYGNWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCGGGGSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 172 - protein, 573 aa, synthetic construct
  REGION 1..573: note = XENP14423 Anti-CD38
EVQLVESGGGLVQPGGSLRLSCAASGFDFSRSWMNWVRQAPGKGLEWVSEINPDSSTINYATSVKGRFTISRDNSKNTLYLQMNSLRAEDTAVYYCARYGNWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 214 aa, synthetic construct; identical to SEQ ID NO: 87
  REGION 1..214: note = XENP14423 Anti-CD38

SEQ ID NO: 174 - protein, 231 aa, synthetic construct; identical to SEQ ID NO: 168
  REGION 1..231: note = XENP15427 Anti-CD38

SEQ ID NO: 175 - protein, 714 aa, synthetic construct
  REGION 1..714: note = XENP15427 Anti-CD38
EVQLVESGGGLVQPGGSLRLSCAASGFDFSRSWMNWVRQAPGKGLEWVSEINPDSSTINYATSVKGRFTISRDNSKNTLYLQMNSLRAEDTAVYYCARYGNWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 176 - protein, 214 aa, synthetic construct; identical to SEQ ID NO: 87
  REGION 1..214: note = XENP15427 Anti-CD38

SEQ ID NO: 177 - protein, 231 aa, synthetic construct; identical to SEQ ID NO: 168
  REGION 1..231: note = XENP15428 Anti-CD38

SEQ ID NO: 178 - protein, 714 aa, synthetic construct
  REGION 1..714: note = XENP15428 Anti-CD38
EVQLVESGGGLVQPGGSLRLSCAASGFDFSRSWMNWVRQAPGKGLEWVSEINPDSSTINYATSVKGRFTISRDNSKNTLYLQMNSLRAEDTAVYYCARYGNWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 179 - protein, 214 aa, synthetic construct; identical to SEQ ID NO: 87
  REGION 1..214: note = XENP15428 Anti-CD38

SEQ ID NO: 180 - protein, 231 aa, synthetic construct; identical to SEQ ID NO: 168
  REGION 1..231: note = XENP15429 Anti-CD38

SEQ ID NO: 181 - protein, 714 aa, synthetic construct; identical to SEQ ID NO: 160
  REGION 1..714: note = XENP15429 Anti-CD38

SEQ ID NO: 182 - protein, 214 aa, synthetic construct; identical to SEQ ID NO: 87
  REGION 1..214: note = XENP15429 Anti-CD38

SEQ ID NO: 183 - protein, 231 aa, synthetic construct; identical to SEQ ID NO: 168
  REGION 1..231: note = XENP15430 Anti-CD38

SEQ ID NO: 184 - protein, 718 aa, synthetic construct
  REGION 1..718: note = XENP15430 Anti-CD38
EVQLVESGGGLVQPGGSLRLSCAASGFDFSRSWMNWVRQAPGKGLEWVSEINPDSSTINYATSVKGRFTISRDNSKNTLYLQMNSLRAEDTAVYYCARYGNWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 214 aa, synthetic construct; identical to SEQ ID NO: 87
  REGION 1..214: note = XENP15430 Anti-CD38

SEQ ID NO: 186 - protein, 231 aa, synthetic construct; identical to SEQ ID NO: 168
  REGION 1..231: note = XENP15431 Anti-CD38

SEQ ID NO: 187 - protein, 718 aa, synthetic construct; identical to SEQ ID NO: 184
  REGION 1..718: note = XENP15431 Anti-CD38

SEQ ID NO: 188 - protein, 214 aa, synthetic construct; identical to SEQ ID NO: 87
  REGION 1..214: note = XENP15431 Anti-CD38

SEQ ID NO: 189 - protein, 231 aa, synthetic construct; identical to SEQ ID NO: 168
  REGION 1..231: note = XENP15432 Anti-CD38

SEQ ID NO: 190 - protein, 718 aa, synthetic construct; identical to SEQ ID NO: 166
  REGION 1..718: note = XENP15432 Anti-CD38

SEQ ID NO: 191 - protein, 214 aa, synthetic construct; identical to SEQ ID NO: 87
  REGION 1..214: note = XENP15432 Anti-CD38

SEQ ID NO: 192 - protein, 445 aa, synthetic construct; identical to SEQ ID NO: 85
  REGION 1..445: note = XENP15433 Anti-CD38

SEQ ID NO: 193 - protein, 714 aa, synthetic construct; identical to SEQ ID NO: 175
  REGION 1..714: note = XENP15433 Anti-CD38

SEQ ID NO: 194 - protein, 214 aa, synthetic construct; identical to SEQ ID NO: 87
  REGION 1..214: note = XENP15433 Anti-CD38

SEQ ID NO: 195 - protein, 445 aa, synthetic construct; identical to SEQ ID NO: 85
  REGION 1..445: note = XENP15434 Anti-CD38

SEQ ID NO: 196 - protein, 714 aa, synthetic construct; identical to SEQ ID NO: 178
  REGION 1..714: note = XENP15434 Anti-CD38

SEQ ID NO: 197 - protein, 214 aa, synthetic construct; identical to SEQ ID NO: 87
  REGION 1..214: note = XENP15434 Anti-CD38

SEQ ID NO: 198 - protein, 445 aa, synthetic construct; identical to SEQ ID NO: 85
  REGION 1..445: note = XENP15435 Anti-CD38

SEQ ID NO: 199 - protein, 714 aa, synthetic construct; identical to SEQ ID NO: 160
  REGION 1..714: note = XENP15435 Anti-CD38

SEQ ID NO: 200 - protein, 214 aa, synthetic construct; identical to SEQ ID NO: 87
  REGION 1..214: note = XENP15435 Anti-CD38

SEQ ID NO: 201 - protein, 445 aa, synthetic construct; identical to SEQ ID NO: 85
  REGION 1..445: note = XENP15436 Anti-CD38

SEQ ID NO: 202 - protein, 718 aa, synthetic construct; identical to SEQ ID NO: 184
  REGION 1..718: note = XENP15436 Anti-CD38

SEQ ID NO: 203 - protein, 214 aa, synthetic construct; identical to SEQ ID NO: 87
  REGION 1..214: note = XENP15436 Anti-CD38

SEQ ID NO: 204 - protein, 445 aa, synthetic construct; identical to SEQ ID NO: 85
  REGION 1..445: note = XENP15437 Anti-CD38

SEQ ID NO: 205 - protein, 718 aa, synthetic construct; identical to SEQ ID NO: 184
  REGION 1..718: note = XENP15437 Anti-CD38

SEQ ID NO: 206 - protein, 214 aa, synthetic construct; identical to SEQ ID NO: 87
  REGION 1..214: note = XENP15437 Anti-CD38

SEQ ID NO: 207 - protein, 445 aa, synthetic construct; identical to SEQ ID NO: 85
  REGION 1..445: note = XENP15438 Anti-CD38

SEQ ID NO: 208 - protein, 718 aa, synthetic construct; identical to SEQ ID NO: 166
  REGION 1..718: note = XENP15438 Anti-CD38

SEQ ID NO: 209 - protein, 214 aa, synthetic construct; identical to SEQ ID NO: 87
  REGION 1..214: note = XENP15438 Anti-CD38

SEQ ID NO: 210 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 211 - protein, 254 aa, synthetic construct; identical to SEQ ID NO: 9
  REGION 1..254: note = anti-CD3 sequences

SEQ ID NO: 212 - protein, 125 aa, synthetic construct; identical to SEQ ID NO: 1
  REGION 1..125: note = anti-CD3 sequences

SEQ ID NO: 213 - protein, 109 aa, synthetic construct; identical to SEQ ID NO: 5
  REGION 1..109: note = anti-CD3 sequences

SEQ ID NO: 214 - protein, 262 aa, synthetic construct; identical to SEQ ID NO: 210
  REGION 1..262: note = anti-CD3 sequences

SEQ ID NO: 215 - protein, 254 aa, synthetic construct; identical to SEQ ID NO: 9
  REGION 1..254: note = anti-CD3 sequences

SEQ ID NO: 216 - protein, 125 aa, synthetic construct; identical to SEQ ID NO: 1
  REGION 1..125: note = anti-CD3 sequences

SEQ ID NO: 217 - protein, 109 aa, synthetic construct; identical to SEQ ID NO: 5
  REGION 1..109: note = anti-CD3 sequences

SEQ ID NO: 218 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 219 - protein, 254 aa, synthetic construct; identical to SEQ ID NO: 45
  REGION 1..254: note = anti-CD3 sequences

SEQ ID NO: 220 - protein, 125 aa, synthetic construct; identical to SEQ ID NO: 37
  REGION 1..125: note = anti-CD3 sequences

SEQ ID NO: 221 - protein, 109 aa, synthetic construct; identical to SEQ ID NO: 5
  REGION 1..109: note = anti-CD3 sequences

SEQ ID NO: 222 - protein, 262 aa, synthetic construct
  REGION 1..262: note = anti-CD3 sequences
EVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 223 - protein, 254 aa, synthetic construct; identical to SEQ ID NO: 54
  REGION 1..254: note = anti-CD3 sequences

SEQ ID NO: 224 - protein, 125 aa, synthetic construct; identical to SEQ ID NO: 46
  REGION 1..125: note = anti-CD3 sequences

SEQ ID NO: 225 - protein, 109 aa, synthetic construct; identical to SEQ ID NO: 5
  REGION 1..109: note = anti-CD3 sequences

SEQ ID NO: 226 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 227 - protein, 254 aa, synthetic construct; identical to SEQ ID NO: 18
  REGION 1..254: note = anti-CD3 sequences

SEQ ID NO: 228 - protein, 125 aa, synthetic construct; identical to SEQ ID NO: 10
  REGION 1..125: note = anti-CD3 sequences

SEQ ID NO: 229 - protein, 109 aa, synthetic construct; identical to SEQ ID NO: 5
  REGION 1..109: note = anti-CD3 sequences

SEQ ID NO: 230 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGPSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 231 - protein, 254 aa, synthetic construct
  REGION 1..254: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGPSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 232 - protein, 125 aa, synthetic construct
  REGION 1..125: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGPSYVSWFAYWGQGTLVTVSS

SEQ ID NO: 233 - protein, 109 aa, synthetic construct; identical to SEQ ID NO: 5
  REGION 1..109: note = anti-CD3 sequences

SEQ ID NO: 234 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 235 - protein, 254 aa, synthetic construct; identical to SEQ ID NO: 27
  REGION 1..254: note = anti-CD3 sequences

SEQ ID NO: 236 - protein, 125 aa, synthetic construct; identical to SEQ ID NO: 19
  REGION 1..125: note = anti-CD3 sequences

SEQ ID NO: 237 - protein, 109 aa, synthetic construct; identical to SEQ ID NO: 5
  REGION 1..109: note = anti-CD3 sequences

SEQ ID NO: 238 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDPYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 239 - protein, 254 aa, synthetic construct; identical to SEQ ID NO: 36
  REGION 1..254: note = anti-CD3 sequences

SEQ ID NO: 240 - protein, 125 aa, synthetic construct; identical to SEQ ID NO: 28
  REGION 1..125: note = anti-CD3 sequences

SEQ ID NO: 241 - protein, 109 aa, synthetic construct; identical to SEQ ID NO: 5
  REGION 1..109: note = anti-CD3 sequences

SEQ ID NO: 242 - protein, 262 aa, synthetic construct; identical to SEQ ID NO: 226
  REGION 1..262: note = anti-CD3 sequences

SEQ ID NO: 243 - protein, 254 aa, synthetic construct; identical to SEQ ID NO: 18
  REGION 1..254: note = anti-CD3 sequences

SEQ ID NO: 244 - protein, 125 aa, synthetic construct; identical to SEQ ID NO: 10
  REGION 1..125: note = anti-CD3 sequences

SEQ ID NO: 245 - protein, 109 aa, synthetic construct; identical to SEQ ID NO: 5
  REGION 1..109: note = anti-CD3 sequences

SEQ ID NO: 246 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKYNNYATAYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 247 - protein, 254 aa, synthetic construct
  REGION 1..254: note = anti-CD3 sequences
EVQLVESGGGLVQPGGSLRLSCAASGFTFSTYAMNWVRQAPGKGLEWVGRIRSKYNNYATAYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 248 - protein, 125 aa, synthetic construct
  REGION 1..125: note = anti-CD3 sequences
EVQLVESGGGLVQPGGSLRLSCAASGFTFSTYAMNWVRQAPGKGLEWVGRIRSKYNNYATAYADSVKGRFTISRDDSKNTLYLQMNSLRAEDTAVYYCVRHGNFGDSYVSWFAYWGQGTLVTVSS

SEQ ID NO: 249 - protein, 109 aa, synthetic construct; identical to SEQ ID NO: 5
  REGION 1..109: note = anti-CD3 sequences

SEQ ID NO: 250 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGESYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 251 - protein, 254 aa, synthetic construct
  REGION 1..254: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGESYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 252 - protein, 125 aa, synthetic construct
  REGION 1..125: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGESYVSWFAYWGQGTLVTVSS

SEQ ID NO: 253 - protein, 109 aa, synthetic construct; identical to SEQ ID NO: 5
  REGION 1..109: note = anti-CD3 sequences

SEQ ID NO: 254 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGQSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 255 - protein, 254 aa, synthetic construct
  REGION 1..254: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGQSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 256 - protein, 125 aa, synthetic construct
  REGION 1..125: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGQSYVSWFAYWGQGTLVTVSS

SEQ ID NO: 257 - protein, 109 aa, synthetic construct; identical to SEQ ID NO: 5
  REGION 1..109: note = anti-CD3 sequences

SEQ ID NO: 258 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDNYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 259 - protein, 254 aa, synthetic construct
  REGION 1..254: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDNYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 260 - protein, 125 aa, synthetic construct
  REGION 1..125: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDNYVSWFAYWGQGTLVTVSS

SEQ ID NO: 261 - protein, 109 aa, synthetic construct; identical to SEQ ID NO: 5
  REGION 1..109: note = anti-CD3 sequences

SEQ ID NO: 262 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDQYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 263 - protein, 254 aa, synthetic construct
  REGION 1..254: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDQYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 264 - protein, 125 aa, synthetic construct
  REGION 1..125: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDQYVSWFAYWGQGTLVTVSS

SEQ ID NO: 265 - protein, 109 aa, synthetic construct; identical to SEQ ID NO: 5
  REGION 1..109: note = anti-CD3 sequences

SEQ ID NO: 266 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 267 - protein, 254 aa, synthetic construct
  REGION 1..254: note = anti-CD3 sequences
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 268 - protein, 125 aa, synthetic construct
  REGION 1..125: note = anti-CD3 sequences
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFDYWGQGTLVTVSS

SEQ ID NO: 269 - protein, 109 aa, synthetic construct; identical to SEQ ID NO: 5
  REGION 1..109: note = anti-CD3 sequences

SEQ ID NO: 270 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKYNNYATAYADSVKGRFTISRDDSKNTLYLQMNSLRAEDTAVYYCVRHGNFGDSYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 271 - protein, 254 aa, synthetic construct
  REGION 1..254: note = anti-CD3 sequences
EVQLVESGGGLVQPGGSLRLSCAASGFTFSTYAMNWVRQAPGKGLEWVGRIRSKYNNYATAYADSVKGRFTISRDDSKNTLYLQMNSLRAEDTAVYYCVRHGNFGDSYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 272 - protein, 125 aa, synthetic construct
  REGION 1..125: note = anti-CD3 sequences
EVQLVESGGGLVQPGGSLRLSCAASGFTFSTYAMNWVRQAPGKGLEWVGRIRSKYNNYATAYADSVKGRFTISRDDSKNTLYLQMNSLRAEDTAVYYCVRHGNFGDSYVSWFDYWGQGTLVTVSS

SEQ ID NO: 273 - protein, 109 aa, synthetic construct; identical to SEQ ID NO: 5
  REGION 1..109: note = anti-CD3 sequences

SEQ ID NO: 274 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGASYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 275 - protein, 254 aa, synthetic construct
  REGION 1..254: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGASYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 276 - protein, 125 aa, synthetic construct
  REGION 1..125: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGASYVSWFDYWGQGTLVTVSS

SEQ ID NO: 277 - protein, 109 aa, synthetic construct; identical to SEQ ID NO: 5
  REGION 1..109: note = anti-CD3 sequences

SEQ ID NO: 278 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGQSYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 279 - protein, 254 aa, synthetic construct
  REGION 1..254: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGQSYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 280 - protein, 125 aa, synthetic construct
  REGION 1..125: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGQSYVSWFDYWGQGTLVTVSS

SEQ ID NO: 281 - protein, 109 aa, synthetic construct; identical to SEQ ID NO: 5
  REGION 1..109: note = anti-CD3 sequences

SEQ ID NO: 282 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 283 - protein, 254 aa, synthetic construct
  REGION 1..254: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 284 - protein, 125 aa, synthetic construct
  REGION 1..125: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFDYWGQGTLVTVSS

SEQ ID NO: 285 - protein, 109 aa, synthetic construct; identical to SEQ ID NO: 5
  REGION 1..109: note = anti-CD3 sequences

SEQ ID NO: 286 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDNYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 287 - protein, 254 aa, synthetic construct
  REGION 1..254: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDNYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 288 - protein, 125 aa, synthetic construct
  REGION 1..125: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDNYVSWFDYWGQGTLVTVSS

SEQ ID NO: 289 - protein, 109 aa, synthetic construct; identical to SEQ ID NO: 5
  REGION 1..109: note = anti-CD3 sequences

SEQ ID NO: 290 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDPYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 291 - protein, 254 aa, synthetic construct
  REGION 1..254: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDPYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 292 - protein, 125 aa, synthetic construct
  REGION 1..125: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDPYVSWFDYWGQGTLVTVSS

SEQ ID NO: 293 - protein, 109 aa, synthetic construct; identical to SEQ ID NO: 5
  REGION 1..109: note = anti-CD3 sequences

SEQ ID NO: 294 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDQYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 295 - protein, 254 aa, synthetic construct
  REGION 1..254: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDQYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 296 - protein, 125 aa, synthetic construct
  REGION 1..125: note = anti-CD3 sequences
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDQYVSWFDYWGQGTLVTVSS

SEQ ID NO: 297 - protein, 109 aa, synthetic construct; identical to SEQ ID NO: 5
  REGION 1..109: note = anti-CD3 sequences

SEQ ID NO: 298 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKANNYATAYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 299 - protein, 254 aa, synthetic construct
  REGION 1..254: note = anti-CD3 sequences
EVQLVESGGGLVQPGGSLRLSCAASGFTFSTYAMNWVRQAPGKGLEWVGRIRSKANNYATAYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 300 - protein, 125 aa, synthetic construct
  REGION 1..125: note = anti-CD3 sequences
EVQLVESGGGLVQPGGSLRLSCAASGFTFSTYAMNWVRQAPGKGLEWVGRIRSKANNYATAYADSVKGRFTISRDDSKNTLYLQMNSLRAEDTAVYYCVRHGNFGDSYVSWFAYWGQGTLVTVSS

SEQ ID NO: 301 - protein, 109 aa, synthetic construct; identical to SEQ ID NO: 5
  REGION 1..109: note = anti-CD3 sequences

SEQ ID NO: 302 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKANNYATAYADSVKGRFTISRDDSKNTLYLQMNSLRAEDTAVYYCVRHGNFGASYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 303 - protein, 254 aa, synthetic construct
  REGION 1..254: note = anti-CD3 sequences
EVQLVESGGGLVQPGGSLRLSCAASGFTFSTYAMNWVRQAPGKGLEWVGRIRSKANNYATAYADSVKGRFTISRDDSKNTLYLQMNSLRAEDTAVYYCVRHGNFGASYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 304 - protein, 125 aa, synthetic construct
  REGION 1..125: note = anti-CD3 sequences
EVQLVESGGGLVQPGGSLRLSCAASGFTFSTYAMNWVRQAPGKGLEWVGRIRSKANNYATAYADSVKGRFTISRDDSKNTLYLQMNSLRAEDTAVYYCVRHGNFGASYVSWFAYWGQGTLVTVSS

SEQ ID NO: 305 - protein, 109 aa, synthetic construct; identical to SEQ ID NO: 5
  REGION 1..109: note = anti-CD3 sequences

SEQ ID NO: 306 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKANNYATAYADSVKGRFTISRDDSKNTLYLQMNSLRAEDTAVYYCVRHGNFGQSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 307 - protein, 254 aa, synthetic construct
  REGION 1..254: note = anti-CD3 sequences
EVQLVESGGGLVQPGGSLRLSCAASGFTFSTYAMNWVRQAPGKGLEWVGRIRSKANNYATAYADSVKGRFTISRDDSKNTLYLQMNSLRAEDTAVYYCVRHGNFGQSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 308 - protein, 125 aa, synthetic construct
  REGION 1..125: note = anti-CD3 sequences
EVQLVESGGGLVQPGGSLRLSCAASGFTFSTYAMNWVRQAPGKGLEWVGRIRSKANNYATAYADSVKGRFTISRDDSKNTLYLQMNSLRAEDTAVYYCVRHGNFGQSYVSWFAYWGQGTLVTVSS

SEQ ID NO: 309 - protein, 109 aa, synthetic construct; identical to SEQ ID NO: 5
  REGION 1..109: note = anti-CD3 sequences

SEQ ID NO: 310 - protein, 262 aa, synthetic construct
  REGION 1..262: note = anti-CD3 sequences
EVQLVESGGGLVQPGGSLRLSCAASGFTFSTYAMNWVRQAPGKGLEWVGRIRSKANNYATAYADSVKGRFTISRDDSKNTLYLQMNSLRAEDTAVYYCVRHGNFGDSYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGSHHHHHH

SEQ ID NO: 311 - protein, 254 aa, synthetic construct
  REGION 1..254: note = anti-CD3 sequences
EVQLVESGGGLVQPGGSLRLSCAASGFTFSTYAMNWVRQAPGKGLEWVGRIRSKANNYATAYADSVKGRFTISRDDSKNTLYLQMNSLRAEDTAVYYCVRHGNFGDSYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 312 - protein, 125 aa, synthetic construct
  REGION 1..125: note = anti-CD3 sequences
EVQLVESGGGLVQPGGSLRLSCAASGFTFSTYAMNWVRQAPGKGLEWVGRIRSKANNYATAYADSVKGRFTISRDDSKNTLYLQMNSLRAEDTAVYYCVRHGNFGDSYVSWFDYWGQGTLVTVSS

SEQ ID NO: 313 - protein, 109 aa, synthetic construct; identical to SEQ ID NO: 5
  REGION 1..109: note = anti-CD3 sequences

SEQ ID NO: 314 - protein, 445 aa, synthetic construct; identical to SEQ ID NO: 82
  REGION 1..445: note = anti-CD3 sequences

SEQ ID NO: 315 - protein, 485 aa, synthetic construct; identical to SEQ ID NO: 104
  REGION 1..485: note = anti-CD3 sequences

SEQ ID NO: 316 - protein, 214 aa, synthetic construct; identical to SEQ ID NO: 84
  REGION 1..214: note = anti-CD3 sequences

SEQ ID NO: 317 - protein, 445 aa, synthetic construct; identical to SEQ ID NO: 85
  REGION 1..445: note = anti-CD3 sequences

SEQ ID NO: 318 - protein, 485 aa, synthetic construct; identical to SEQ ID NO: 104
  REGION 1..485: note = anti-CD3 sequences

SEQ ID NO: 319 - protein, 214 aa, synthetic construct; identical to SEQ ID NO: 87
  REGION 1..214: note = anti-CD3 sequences

SEQ ID NO: 320 - protein, 445 aa, synthetic construct; identical to SEQ ID NO: 85
  REGION 1..445: note = anti-CD3 sequences

SEQ ID NO: 321 - protein, 485 aa, synthetic construct; identical to SEQ ID NO: 92
  REGION 1..485: note = anti-CD3 sequences

SEQ ID NO: 322 - protein, 214 aa, synthetic construct; identical to SEQ ID NO: 84
  REGION 1..214: note = anti-CD3 sequences

SEQ ID NO: 323 - protein, 445 aa, synthetic construct; identical to SEQ ID NO: 85
  REGION 1..445: note = anti-CD3 sequences

SEQ ID NO: 324 - protein, 485 aa, synthetic construct; identical to SEQ ID NO: 89
  REGION 1..485: note = anti-CD3 sequences

SEQ ID NO: 325 - protein, 214 aa, synthetic construct; identical to SEQ ID NO: 84
  REGION 1..214: note = anti-CD3 sequences

SEQ ID NO: 326 - protein, 445 aa, synthetic construct; identical to SEQ ID NO: 85
  REGION 1..445: note = anti-CD3 sequences

SEQ ID NO: 327 - protein, 485 aa, synthetic construct; identical to SEQ ID NO: 92
  REGION 1..485: note = anti-CD3 sequences

SEQ ID NO: 328 - protein, 214 aa, synthetic construct; identical to SEQ ID NO: 87
  REGION 1..214: note = anti-CD3 sequences

SEQ ID NO: 329 - protein, 445 aa, synthetic construct; identical to SEQ ID NO: 85
  REGION 1..445: note = anti-CD3 sequences

SEQ ID NO: 330 - protein, 485 aa, synthetic construct; identical to SEQ ID NO: 89
  REGION 1..485: note = anti-CD3 sequences

SEQ ID NO: 331 - protein, 214 aa, synthetic construct; identical to SEQ ID NO: 87
  REGION 1..214: note = anti-CD3 sequences

SEQ ID NO: 332 - protein, 5 aa, synthetic construct
  REGION 1..5: note = useful linkers
GSGGS

SEQ ID NO: 333 - protein, 5 aa, synthetic construct
  REGION 1..5: note = useful linkers
GGGGS

SEQ ID NO: 334 - protein, 4 aa, synthetic construct
  REGION 1..4: note = useful linkers
GGGS

SEQ ID NO: 335 - protein, 485 aa, synthetic construct; identical to SEQ ID NO: 104
  REGION 1..485: note = XmAb18968 (412220) ScFc

SEQ ID NO: 336 - protein, 214 aa, synthetic construct; identical to SEQ ID NO: 84
  REGION 1..214: note = XmAb18968 (412220) LC

SEQ ID NO: 337 - protein, 445 aa, synthetic construct; identical to SEQ ID NO: 85
  REGION 1..445: note = XmAb18968 (412220) HC

SEQ ID NO: 338 - DNA, 1449 bp, synthetic construct
  misc_feature 1..1449: note = XmAb18968 (412220) ScFv-Fc
  source 1..1449: mol_type = other DNA
cagctcgtcgagtctggaggtggcctcgtccagccaggcggtagtctgaggctctcatgcgctgccagcggattcacttttagcacatatgcgatgaattgggtcagacaggctccaggcaaagggctggagtgggtgggacggatcagaagtaagtataataattatgccacctattatgctgactccgtgaagggcagattcactatttctagggacgattccaagaacactctctacctgcaaatgaattccctgagggctgaggacaccgcggtctactattgcgtgcggcacggcaacttcggcgactcttacgtgtcttggttcgactattgggggcaagggactctcgtgacagtgagctccggtaagcctggtagcgggaaaccaggcagcggaaagcctggcagcggtaaaccaggctcccaggccgtggttacacaggaaccctccttgactgtgtcacctgggggcacagtcactcttacctgcggcagttccactggggccgtaacaacctctaactatgcgaattgggttcagcagaagccaggtaagagccctagggggttgataggtggaactaataaacgggcccccggggtgccagcccgcttcagtgggagtttgctggggggaaaggccgccctcaccatctcaggggctcaacccgaggatgaggcagactattactgcgccctgtggtactccaatcactgggtcttcggcgggggtacaaagctgactgtgctggagcccaaatctagcgacaaaactcacacatgcccaccgtgcccggcgcctcctgttgctggaccgtcagtcttcctcttccccccaaaacccaaggacaccctcatgatctcccggacccctgaggtcacatgcgtggtggtggacgtgaagcacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtataccctgcccccatcccgggagcagatgaccaagaaccaggtaaagttgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 339 - DNA, 642 bp, synthetic construct
  misc_feature 1..642: note = XmAb18968 (412220) LC
  source 1..642: mol_type = other DNA
gacatcgtgatgacacagagtcccagctctctctcagcgagcgttggcgatagggtgactataacatgcagagcatcccaaaacgtggatacatgggtggcttggtaccaacagaagccaggtcagtctcccaaggccctgatttatagcgcatcctatcgctacagtggtgttcccgacaggtttaccgggtctggcagtggcaccgattttaccctgactatctcttccctgcaacccgaggacttcgcaacatatttctgccagcagtatgattcctaccccctgaccttcggcggaggcaccaaactcgaaattaagcgtacggtggccgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 340 - DNA, 1329 bp, synthetic construct
  misc_feature 1..1329: note = XmAb18968 (412220) LC
  source 1..1329: mol_type = other DNA
cagttggtggagagcgggggcggcctcgtccagccggggggctcactgaggctctcctgtgcagccagcggattcgacttcagccgatcatggatgaattgggtgagacaggcacctggtaagggccttgagtgggtctctgagattaatccagactctagtaccattaattacgctacttcagtcaagggcaggtttacgatctcaagagacaacagcaagaatacgctgtacctccaaatgaactctcttagggctgaagatactgcagtgtactactgtgcacgatatgggaactggtttccgtattggggccagggcacgttggtgaccgtgtcttccgcaagcactaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcgacaccaaggtggacaagaaagttgagcccaaatcttgtgacaaaactcacacatgcccaccgtgcccggcgcctcctgttgctggaccgtcagtcttcctcttccccccaaaacccaaggacaccctcatgatctcccggacccctgaggtcacatgcgtggtggtggacgtgaagcacgaagaccctgaggtcaagttcaactggtacgtggacggcgtggaggtgcataatgccaagacaaagccgcgggaggaggagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtataccctgcccccatcccgggaggagatgaccaagaaccaggtaagcttgacctgcgacgtcagcggcttctatcccagcgacatcgccgtggagtgggagagcgacgggcagccggagaacaactacaagaccacgcctcccgtgctggactccgacggctccttcttcctctatagcaagctcaccgtggacaagagcaggtgggagcagggggacgtcttctcatgctccgtgatgcatgaggctctgcacaaccactacacgcagaagagcctctccctgtctccgggtaaa

SEQ ID NO: 341 - protein, 504 aa, synthetic construct
  REGION 1..504: note = XmAb18968 (412220) ScFv-Fc
MGWSCIILFLVATATGVHSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 342 - protein, 233 aa, synthetic construct
  REGION 1..233: note = XmAb18968 (412220) LC
MGWSCIILFLVATATGVHSDIVMTQSPSSLSASVGDRVTITCRASQNVDTWVAWYQQKPGQSPKALIYSASYRYSGVPDRFTGSGSGTDFTLTISSLQPEDFATYFCQQYDSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 343 - protein, 464 aa, synthetic construct
  REGION 1..464: note = XmAb18968 (412220) HC
MGWSCIILFLVATATGVHSEVQLVESGGGLVQPGGSLRLSCAASGFDFSRSWMNWVRQAPGKGLEWVSEINPDSSTINYATSVKGRFTISRDNSKNTLYLQMNSLRAEDTAVYYCARYGNWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 344 - DNA, 1512 bp, synthetic construct
  misc_feature 1..1512: note = XmAb18968 (412220) ScFv-Fc
  source 1..1512: mol_type = other DNA
atgggatggagctgtatcatcctcttcttggtagcaacagctacaggtgtacactccgaagttcagctcgtcgagtctggaggtggcctcgtccagccaggcggtagtctgaggctctcatgcgctgccagcggattcacttttagcacatatgcgatgaattgggtcagacaggctccaggcaaagggctggagtgggtgggacggatcagaagtaagtataataattatgccacctattatgctgactccgtgaagggcagattcactatttctagggacgattccaagaacactctctacctgcaaatgaattccctgagggctgaggacaccgcggtctactattgcgtgcggcacggcaacttcggcgactcttacgtgtcttggttcgactattgggggcaagggactctcgtgacagtgagctccggtaagcctggtagcgggaaaccaggcagcggaaagcctggcagcggtaaaccaggctcccaggccgtggttacacaggaaccctccttgactgtgtcacctgggggcacagtcactcttacctgcggcagttccactggggccgtaacaacctctaactatgcgaattgggttcagcagaagccaggtaagagccctagggggttgataggtggaactaataaacgggcccccggggtgccagcccgcttcagtgggagtttgctggggggaaaggccgccctcaccatctcaggggctcaacccgaggatgaggcagactattactgcgccctgtggtactccaatcactgggtcttcggcgggggtacaaagctgactgtgctggagcccaaatctagcgacaaaactcacacatgcccaccgtgcccggcgcctcctgttgctggaccgtcagtcttcctcttccccccaaaacccaaggacaccctcatgatctcccggacccctgaggtcacatgcgtggtggtggacgtgaagcacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtataccctgcccccatcccgggagcagatgaccaagaaccaggtaaagttgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 345 - DNA, 699 bp, synthetic construct
  misc_feature 1..699: note = XmAb18968 (412220) LC
  source 1..699: mol_type = other DNA
atgggatggagctgtatcatcctcttcttggtagcaacagctacaggtgtccacagcgacatcgtgatgacacagagtcccagctctctctcagcgagcgttggcgatagggtgactataacatgcagagcatcccaaaacgtggatacatgggtggcttggtaccaacagaagccaggtcagtctcccaaggccctgatttatagcgcatcctatcgctacagtggtgttcccgacaggtttaccgggtctggcagtggcaccgattttaccctgactatctcttccctgcaacccgaggacttcgcaacatatttctgccagcagtatgattcctaccccctgaccttcggcggaggcaccaaactcgaaattaagcgtacggtggccgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 346 - DNA, 1392 bp, synthetic construct
  misc_feature 1..1392: note = XmAb18968 (412220) LC
  source 1..1392: mol_type = other DNA
atgggatggagctgtatcatcctcttcttggtagcaacagctacaggtgtccactccgaggttcagttggtggagagcgggggcggcctcgtccagccggggggctcactgaggctctcctgtgcagccagcggattcgacttcagccgatcatggatgaattgggtgagacaggcacctggtaagggccttgagtgggtctctgagattaatccagactctagtaccattaattacgctacttcagtcaagggcaggtttacgatctcaagagacaacagcaagaatacgctgtacctccaaatgaactctcttagggctgaagatactgcagtgtactactgtgcacgatatgggaactggtttccgtattggggccagggcacgttggtgaccgtgtcttccgcaagcactaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcgacaccaaggtggacaagaaagttgagcccaaatcttgtgacaaaactcacacatgcccaccgtgcccggcgcctcctgttgctggaccgtcagtcttcctcttccccccaaaacccaaggacaccctcatgatctcccggacccctgaggtcacatgcgtggtggtggacgtgaagcacgaagaccctgaggtcaagttcaactggtacgtggacggcgtggaggtgcataatgccaagacaaagccgcgggaggaggagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtataccctgcccccatcccgggaggagatgaccaagaaccaggtaagcttgacctgcgacgtcagcggcttctatcccagcgacatcgccgtggagtgggagagcgacgggcagccggagaacaactacaagaccacgcctcccgtgctggactccgacggctccttcttcctctatagcaagctcaccgtggacaagagcaggtgggagcagggggacgtcttctcatgctccgtgatgcatgaggctctgcacaaccactacacgcagaagagcctctccctgtctccgggtaaa

SEQ ID NO: 347 - protein, 329 aa, synthetic construct
  REGION 1..329: note = Fab side HC (Bottle opener backbone 1)
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 348 - protein, 231 aa, synthetic construct
  REGION 1..231: note = scFv HC (Bottle opener backbone 1)
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 349 - protein, 107 aa, synthetic construct [sequence omitted]

SEQ ID NO: 350 - protein, 231 aa, synthetic construct
  REGION 1..231: note = scFv HC (Bottle opener backbone 2)
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 351 - protein, 329 aa, synthetic construct
  REGION 1..329: note = Fab side HC (Bottle opener backbone 3)
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCEVSGFYPSDIAVEWESDGQPENNYKTTPPVLDSDGSFFLYSKLTVDKSRWEQGDVFSCSVMHEALHNHYTQKSLSLSPGK

SEQ ID NO: 352 - protein, 231 aa, synthetic construct; identical to SEQ ID NO: 350
  REGION 1..231: note = scFv HC (Bottle opener backbone 3)

SEQ ID NO: 353 - protein, 330 aa, synthetic construct
  REGION 1..330: note = Fab side HC (Bottle opener backbone 4)
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTENEVSLTCLVKSGFYPSDIAVEWESDGQPENNYKTTPPVLDSDGSFFLYSKLEVDKSRWEQGDVFSCSVMHEALHNHYTQKSLSLSPGK

SEQ ID NO: 354 - protein, 231 aa, synthetic construct
  REGION 1..231: note = scFv HC (Bottle opener backbone 4)
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSKGSFFLYSKLTVDKSRWQQGNVFSCSVMHEALHNHYTQKSLSLSPGK

SEQ ID NO: 355 - protein, 107 aa, synthetic construct
  REGION 1..107: note = Variable light (vl) domain (H1L1) CD38 arm
DIVMTQSPSSLSASVGDRVTITCRASQNVDTNVAWYQQKPGQSPKALIYSASYRYSGVPDRFTGSGSGTDFTLTISSLQPEDFATYFCQQYDSYPLTFGGGTKLEIK

SEQ ID NO: 356 - protein, 339 aa, Homo sapiens
MESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEICSQL

SEQ ID NO: 357 - protein, 10 aa, synthetic construct
  REGION 1..10: note = Synthetic Polypeptide
RASSSVSYMH

SEQ ID NO: 358 - protein, 7 aa, synthetic construct
  REGION 1..7: note = Synthetic Polypeptide
STSNLAS

SEQ ID NO: 359 - protein, 9 aa, synthetic construct
  REGION 1..9: note = Synthetic Polypeptide
QQRRSFPYT

SEQ ID NO: 360 - protein, 5 aa, synthetic construct
  REGION 1..5: note = Synthetic Polypeptide
TYWIE

SEQ ID NO: 361 - protein, 17 aa, synthetic construct
  REGION 1..17: note = Synthetic Polypeptide
EILPGSGNTDFNEKFQG

SEQ ID NO: 362 - protein, 12 aa, synthetic construct
  REGION 1..12: note = Synthetic Polypeptide
WGYYGTRGYFNV

SEQ ID NO: 363 - protein, 17 aa, synthetic construct
  REGION 1..17: note = Synthetic Polypeptide
EILPGSGQTDFNEKFQG

SEQ ID NO: 364 - protein, 232 aa, synthetic construct
  REGION 1..232: note = Synthetic Polypeptide
MGWSCIILFLVATATGVHSEIVLTQSPATLSLSPGERATLSCRASSSVSYMHWFQQKPGQAPRLLIYSTSNLASGIPARFSGSGSGTDYTLTISSLEPEDFAVYYCQQRRSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 365 - protein, 469 aa, synthetic construct
  REGION 1..469: note = Synthetic Polypeptide
MGWSCIILFLVATATGVHSQVQLVQSGAEVKKPGASVKVSCKASGYTFSTYWIEWVRQAPGQRLEWMGEILPGSGNTDFNEKFQGRVTFTADTSSDTAYMELSSLRSEDTAVYYCTRWGYYGTRGYFNVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 366 - protein, 742 aa, synthetic construct
  REGION 1..742: note = Synthetic Polypeptide
MGWSCIILFLVATATGVHSQVQLVQSGAEVKKPGASVKVSCKASGYTFSTYWIEWVRQAPGQRLEWMGEILPGSGNTDFNEKFQGRVTFTADTSSDTAYMELSSLRSEDTAVYYCTRWGYYGTRGYFNVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 367 - protein, 745 aa, synthetic construct
  REGION 1..745: note = Synthetic Polypeptide
MDMRVPAQLLGLLLLWLRGARCQVQLVQSGAEVKKPGASVKVSCKASGYTFSTYWIEWVRQAPGQRLEWMGEILPGSGQTDFNEKFQGRVTFTADTSSDTAYMELSSLRSEDTAVYYCTRWGYYGTRGYFNVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGSGGGGSKTHTCPPCPAPPVAGPSVFLFPPKPKDTLMISRTPEVTCVVVDVKHEDPEVKFNWYVDGVEVHNAKTKPCEEQYGSTYRCVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 368 - protein, 5 aa, synthetic construct
  REGION 1..5: note = Synthetic Polypeptide
NYGMN

SEQ ID NO: 369 - protein, 17 aa, synthetic construct
  REGION 1..17: note = Synthetic Polypeptide
WMNTYTGEPTYADKFQG

SEQ ID NO: 370 - protein, 12 aa, synthetic construct
  REGION 1..12: note = Synthetic Polypeptide
AGGQLRPGAMDY

SEQ ID NO: 371 - protein, 15 aa, synthetic construct
  REGION 1..15: note = Synthetic Polypeptide
KASQSVDYDGDSFMN

SEQ ID NO: 372 - protein, 7 aa, synthetic construct
  REGION 1..7: note = Synthetic Polypeptide
VASNLES

SEQ ID NO: 373 - protein, 9 aa, synthetic construct
  REGION 1..9: note = Synthetic Polypeptide
QQSNEEPPT

SEQ ID NO: 374 - protein, 237 aa, synthetic construct
  REGION 1..237: note = Synthetic Polypeptide
MGWSCIILFLVATATGVHSDIVLTQTPLSLSVTPGQPASISCKASQSVDYDGDSFMNWYLQKPGQPPQLLIYVASNLESGVPDRFSGSGSGTDFTLKISRVEAEDVGVYYCQQSNEEPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 375 - protein, 469 aa, synthetic construct
  REGION 1..469: note = Synthetic Polypeptide
MGWSCIILFLVATATGVHSEIQLVQSGAEVKKPGATVKISCKASGYTFTNYGMNWVQQAPGQGLEWMGWMNTYTGEPTYADKFQGRVTFTLDTSARTVYMELSSLRSEDTAVYFCARAGGQLRPGAMDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 376 - protein, 742 aa, synthetic construct
  REGION 1..742: note = Synthetic Polypeptide
MGWSCIILFLVATATGVHSEIQLVQSGAEVKKPGATVKISCKASGYTFTNYGMNWVQQAPGQGLEWMGWMNTYTGEPTYADKFQGRVTFTLDTSARTVYMELSSLRSEDTAVYFCARAGGQLRPGAMDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 377 - protein, 121 aa, synthetic construct
  REGION 1..121: note = Synthetic Polypeptide
QVQLVQSGAEVKKPGASVKVSCKASGYTFSTYWIEWVRQAPGQRLEWMGEILPGSGNTDFNEKFQGRVTFTADTSSDTAYMELSSLRSEDTAVYYCTRWGYYGTRGYFNVWGQGTLVTVSS

SEQ ID NO: 378 - protein, 106 aa, synthetic construct [sequence omitted]

SEQ ID NO: 379 - protein, 121 aa, synthetic construct [sequence omitted]

SEQ ID NO: 380 - protein, 121 aa, synthetic construct
  REGION 1..121: note = Synthetic Polypeptide
EIQLVQSGAEVKKPGATVKISCKASGYTFTNYGMNWVQQAPGQGLEWMGWMNTYTGEPTYADKFQGRVTFTLDTSARTVYMELSSLRSEDTAVYFCARAGGQLRPGAMDYWGQGTMVTVSS

SEQ ID NO: 381 - protein, 111 aa, synthetic construct
  REGION 1..111: note = Synthetic Polypeptide
DIVLTQTPLSLSVTPGQPASISCKASQSVDYDGDSFMNWYLQKPGQPPQLLIYVASNLESGVPDRFSGSGSGTDFTLKISRVEAEDVGVYYCQQSNEEPPTFGQGTKLEIK

SEQ ID NO: 382 - protein, 125 aa, synthetic construct; identical to SEQ ID NO: 1
  REGION 1..125: note = Synthetic Polypeptide

SEQ ID NO: 383 - protein, 5 aa, synthetic construct
  REGION 1..5: note = Synthetic Polypeptide
TYAMN

SEQ ID NO: 384 - protein, 19 aa, synthetic construct
  REGION 1..19: note = Synthetic Polypeptide
RIRSKYNNYATYYADSVKG

SEQ ID NO: 385 - protein, 14 aa, synthetic construct
  REGION 1..14: note = Synthetic Polypeptide
HGNFGDSYVSWFAY

SEQ ID NO: 386 - protein, 109 aa, synthetic construct; identical to SEQ ID NO: 5
  REGION 1..109: note = Synthetic Polypeptide

SEQ ID NO: 387 - protein, 14 aa, synthetic construct
  REGION 1..14: note = Synthetic Polypeptide
GSSTGAVTTSNYAN

SEQ ID NO: 388 - protein, 7 aa, synthetic construct
  REGION 1..7: note = Synthetic Polypeptide
GTNKRAP

SEQ ID NO: 389 - protein, 9 aa, synthetic construct
  REGION 1..9: note = Synthetic Polypeptide
ALWYSNHWV

SEQ ID NO: 390 - protein, 254 aa, synthetic construct; identical to SEQ ID NO: 9
  REGION 1..254: note = Synthetic Polypeptide

SEQ ID NO: 391 - protein, 20 aa, synthetic construct
  REGION 1..20: note = Synthetic Polypeptide
GKPGSGKPGSGKPGSGKPGS